(19) United States
(12) Patent Application Publication
(10) Pub. No.: US 2024/0027903 A1
(43) Pub. Date: Jan. 25, 2024

(54) Resist Material And Patterning Process

(71) Applicant: SHIN-ETSU CHEMICAL CO., LTD., Tokyo (JP)

(72) Inventors: Jun HATAKEYAMA, Joetsu-shi (JP); Masahiro FUKUSHIMA, Joetsu-shi (JP)

(73) Assignee: SHIN-ETSU CHEMICAL CO., LTD., Tokyo (JP)

(21) Appl. No.: 18/349,633

(22) Filed: Jul. 10, 2023

(30) Foreign Application Priority Data
Jul. 12, 2022 (JP) 2022-111944
Feb. 8, 2023 (JP) 2023-17434

(51) Int. Cl. G03F 7/004; G03F 7/039; G03F 7/038; C07C 65/03; C07C 65/21; C07C 311/08; C07C 321/28; C07C 69/96; G03F 7/20

(52) U.S. Cl. CPC G03F 7/0045; G03F 7/0397; G03F 7/0382; G03F 7/0388; C07C 65/03; C07C 65/21; C07C 311/08; C07C 321/28; C07C 69/96; G03F 7/2037

(57) ABSTRACT

Provided are: a resist material having high sensitivity and improved LWR (line width roughness) and CDU (critical dimension uniformity) for both of positive-type and negative-type resists; and a patterning process using this resist material: a resist material, including, as a quencher: a sulfonium salt of a substituted or unsubstituted hydroxy(trifluoromethoxy)benzoic acid; and/or a sulfonium salt of a substituted or unsubstituted hydroxy(trifluoromethylthio)benzoic acid.

### TECHNICAL FIELD

The present invention relates to a resist material and a patterning process.

### BACKGROUND ART

As higher integration and higher speed of LSI have been achieved, a pattern rule has been rapidly miniaturized. This is because a high-speed communication with 5G and artificial intelligence (AI) have become widespread, and a high-performance device to process them has been required. As the latest microfabrication technique, 5-nm node devices are industrially manufactured using lithography with extreme ultraviolet ray (EUV) having a wavelength of 13.5 nm. Furthermore, investigation using the EUV lithography is progressed for a 3-nm node device, next generation, and a 2-nm node device, next to the next generation.

The miniaturization in progress causes a problem of blurring an image due to acid diffusion. To achieve resolution with a fine pattern of 45 nm or finer in size, not only improvement of a dissolution contrast, conventionally proposed, but also importance of controlling the acid diffusion is proposed (Non Patent Document 1). However, since a chemically amplified resist material enhances sensitivity and contrast by utilizing the acid diffusion, inhibiting the acid diffusion to the utmost limit with lowering a temperature or shortening a time of post exposure bake (PEB) considerably deteriorates the sensitivity and the contrast.

Pointed out is a triangle trade-off relationship between sensitivity, resolution, and edge roughness. Although inhibiting the acid diffusion is required in order to improve the resolution, shortening a distance of the acid diffusion deteriorates the sensitivity.

It is effective that an acid generator to generate a bulky acid is added to inhibit the acid diffusion. Accordingly, proposed is containing a repeating unit derived from an onium salt having a polymerizable unsaturated bond into a polymer. In this case, the polymer also functions as an acid generator (polymer-bound acid generator). Patent Document 1 proposes a sulfonium salt or iodonium salt having a polymerizable unsaturated bond to generate a specific sulfonic acid. Patent Document 2 proposes a sulfonium salt in which a sulfonic acid is directly bonded to a main chain.

With an acid-labile group used for a (meth)acrylate polymer for an ArF resist material, using a photoacid generator to generate a sulfonic acid having an α-position substituted with a fluorine atom proceeds a deprotection reaction. An acid generator to generate a sulfonic acid or a carboxylic acid having an α-position not substituted with a fluorine atom does not proceed the deprotection reaction. Mixing the sulfonium salt or iodonium salt to generate a sulfonic acid having an α-position not substituted with a fluorine atom with the sulfonium salt or iodonium salt to generate the sulfonic acid having an α-position substituted with a fluorine atom proceeds ion exchange between the sulfonium salt or iodonium salt to generate the sulfonic acid having an α-position not substituted with a fluorine atom and the sulfonic acid having an α-position substituted with a fluorine atom. The photo-generated sulfonic acid having an α-position substituted with a fluorine atom is returned to the sulfonium salt or the iodonium salt with the ion exchange, and thereby the sulfonium salt or iodonium salt of the sulfonic acid or a carboxylic acid having an α-position not substituted with a fluorine atom functions as a quencher. Proposed is a resist material using a sulfonium salt or iodonium salt to generate a carboxylic acid as a quencher (Patent Document 3).

Various sulfonium salt quenchers to generate a carboxylic acid are proposed. Specifically proposed are sulfonium salts of salicylic acid or a (3-hydroxycarboxylic acid (Patent Document 4), a salicylic acid derivative (Patent Documents 5 and 6), fluorosalicylic acid (Patent Document 7), and hydroxynaphthoic acid (Patent Document 8). In particular, salicylic acid has an effect of inhibiting the acid diffusion with an intramolecular hydrogen bond between the carboxylic acid and the hydroxy group.

Meanwhile, it is pointed out that aggregation of the quencher deteriorates critical dimension uniformity of a resist pattern. Preventing the aggregation of the quencher in a resist film to uniformize the distribution is promising to increase the critical dimension uniformity of a pattern after development.

A trifluoromethoxy group, which has a strong electron-withdrawing effect, is called “super halogen”, and used for medicines, agricultural chemicals, and liquid crystal materials. As the anion of a sulfonium salt for a resist, 4-trifluoromethoxybenzoic acid is described (Patent Document 9).

### CITATION LIST

### Patent Literature

Patent Document 1: JP 2006-045311 A
Patent Document 2: JP 2006-178317 A
Patent Document 3: JP2007-114431 A
Patent Document 4: WO 2018/159560
Patent Document 5: JP 2020-203984 A
Patent Document 6: JP 2020-91404 A
Patent Document 7: WO 2010/095698
Patent Document 8: JP 2019-120760 A
Patent Document 9: JP2007-284405 A

### Non Patent Literature

Non Patent Document 1: SPIE Vol. 6520 65203L-1 (2007)

### SUMMARY OF INVENTION

### Technical Problem

The present invention has been made in view of the above circumstances. An object of the present invention is to provide: a resist material having high sensitivity and improved LWR (line width roughness) and CDU (critical dimension uniformity) for both of positive-type and negative-type resists; and a patterning process using this resist material.

### Solution to Problem

To solve the above problem, the present invention provides a resist material comprising, as a quencher: a sulfonium salt of a substituted or unsubstituted hydroxy(trifluoromethoxy)benzoic acid; and/or a sulfonium salt of a substituted or unsubstituted hydroxy(trifluoromethylthio)benzoic acid.

Such a resist material has high sensitivity and improved LWR and CDU for both of positive-type and negative-type resists.

The sulfonium salt of the substituted or unsubstituted hydroxy(trifluoromethoxy)benzoic acid and/or the sulfonium salt of the substituted or unsubstituted hydroxy(trifluoromethylthio)benzoic acid is preferably represented by the following general formula (1),

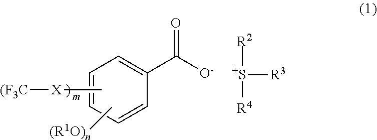

wherein R¹ represents a hydrogen atom or a group selected from a linear, branched, or cyclic alkyl group, alkoxy group, alkoxycarbonyl group, or acyl group having 1 to 15 carbon atoms, an alkenyl group having 2 to 15 carbon atoms, and an alkynyl group having 2 to 15 carbon atoms, the group optionally having a halogen atom, a carbonyl group, an ether bond, a substituted or unsubstituted aryl group, or a hydroxy group, the group being optionally an acid-labile group; X represents an oxygen atom or a sulfur atom; “m” and “n” each represent 1 or 2; and R² to R⁴ each independently represent a halogen atom or a hydrocarbyl group having 1 to 25 carbon atoms and optionally having a heteroatom, R² and R³ being optionally bonded to each other to form a ring together with the sulfur atom to which these groups are bonded.

Such a resist material can more certainly have high sensitivity and improved LWR and CDU.

The present invention also provides a resist material comprising, as a quencher, one or more of: a sulfonium salt of a substituted or unsubstituted hydroxy(difluoromethoxy)benzoic acid; a sulfonium salt of a substituted or unsubstituted hydroxy(difluoromethylthio)benzoic acid; a sulfonium salt of a 2- or 3-, di- or tri-fluoromethoxybenzoic acid; and a sulfonium salt of a 2- or 3-, di- or tri-fluoromethylthiobenzoic acid.

Such a resist material has high sensitivity and improved LWR and CDU for both of positive-type and negative-type resists.

One or more of the sulfonium salt of the substituted or unsubstituted hydroxy(difluoromethoxy)benzoic acid, the sulfonium salt of the substituted or unsubstituted hydroxy(difluoromethylthio)benzoic acid, the sulfonium salt of the 2- or 3-, di- or tri-fluoromethoxybenzoic acid, and the sulfonium salt of the 2- or 3-, di- or tri-fluoromethylthiobenzoic acid are represented by the following general formula (1′),

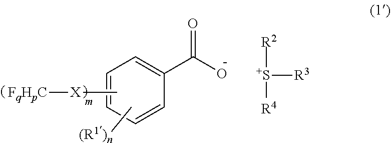

wherein R^{1′} represents a hydrogen atom, a halogen atom, or a group selected from a hydroxy group, a substituted or unsubstituted amino group, a linear, branched, or cyclic alkyl group, alkoxy group, alkoxycarbonyl group, alkoxycarbonyloxy group, acyl group, or acyloxy group having 1 to 15 carbon atoms, an alkenyloxy group having 2 to 15 carbon atoms, and an alkynyloxy group having 2 to 15 carbon atoms, the group optionally having a halogen atom, a carbonyl group, an ether bond, a substituted or unsubstituted aryl group, or a hydroxy group, the group being optionally an acid-labile group, and when R^{1′} represents a hydrogen atom, the substituting position of the F_{q}HₚC—X— group is the 2- or 3-position; “p” represents 0 or 1; “q” represents 2 or 3; X represents an oxygen atom or a sulfur atom; “m” and “n” each represent 1 or 2; and R² to R⁴ each independently represent a halogen atom or a hydrocarbyl group having 1 to 25 carbon atoms and optionally having a heteroatom, R² and R³ being optionally bonded to each other to form a ring together with the sulfur atom to which these groups are bonded.

Such a resist material can more certainly have high sensitivity and improved LWR and CDU.

The resist material preferably further comprises an acid generator to generate an acid.

In such a resist material, the above sulfonium salt functions as a quencher to allow the inventive resist material to function.

The acid generator preferably generates a sulfonic acid, an imide acid, or a methide acid.

Such a photoacid generator is more preferable.

The resist material preferably further comprises an organic solvent.

Such a resist material is preferably used, dissolving each contained component.

The resist material preferably further comprises a base polymer.

Such a resist material is preferable.

The base polymer preferably comprises a repeating unit represented by the following general formula (a1) and/or a repeating unit represented by the following general formula (a2),

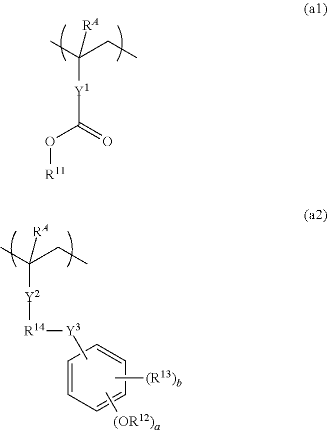

wherein R^{A} each independently represents a hydrogen atom or a methyl group; Y¹ represents a single bond, a phenylene group, a naphthylene group, or a linking group having 1 to 12 carbon atoms and having at least one selected from an ester bond and a lactone ring; Y² represents a single bond or an ester bond; Y³ represents a single bond, an ether bond, or an ester bond; R¹¹ and R¹² each independently represent an acid-labile group; R¹³ represents a fluorine atom, a trifluoromethyl group, a cyano group, or a saturated hydrocarbyl group having 1 to 6 carbon atoms; R¹⁴ represents a single bond or an alkanediyl group having 1 to 6 carbon atoms, a part of carbon atoms therein being optionally substituted with an ether bond or an ester bond; “a” represents 1 or 2; “b” represents an integer of 0 to 4, and 1≤a+b≤5.

Such a resist material has an acid-labile group, and suitable as a positive-type resist material.

The resist material is preferably a chemically amplified positive-type resist material.

The inventive resist material can function as a chemically amplified positive-type resist material.

The base polymer also preferably has no acid-labile group.

Such a resist material has no acid-labile group, and suitable as a negative-type resist material.

The resist material is preferably a chemically amplified negative-type resist material.

The inventive resist material can function as a chemically amplified negative-type resist material.

The base polymer preferably further comprises at least one selected from repeating units represented by the following general formulae (f1) to (f3),

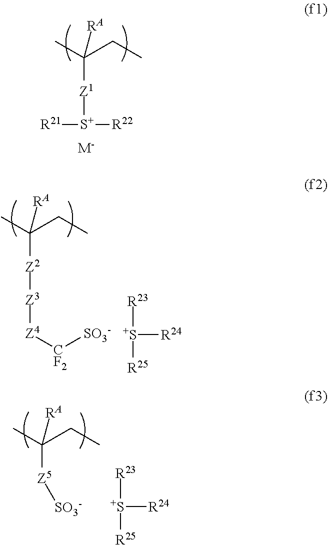

wherein R^{A} each independently represents a hydrogen atom or a methyl group; Z¹ represents a single bond, an aliphatic hydrocarbylene group having 1 to 6 carbon atoms, a phenylene group, a naphthylene group, an ester bond, a group having 7 to 18 carbon atoms obtained by combining these groups, —O—Z¹¹—, —C(═O)—O—Z¹¹—, or —C(═O)—NH—Z¹¹—; Z¹¹ represents an aliphatic hydrocarbylene group having 1 to 6 carbon atoms, a phenylene group, a naphthylene group, or a group having 7 to 18 carbon atoms obtained by combining these groups, Z¹¹ optionally having a carbonyl group, an ester bond, an ether bond, or a hydroxy group; Z² represents a single bond or an ester bond; Z³ represents a single bond, —Z³¹—C(═O)—O—, —Z³¹—O—, or —Z³¹—O—C(═O)—; Z³¹ represents a hydrocarbylene group having 1 to 12 carbon atoms, a phenylene group, or a group having 7 to 18 carbon atoms obtained by combining these groups, Z³¹ optionally having a carbonyl group, an ester bond, an ether bond, an iodine atom, or a bromine atom; Z⁴ represents a methylene group, a 2,2,2-trifluoro-1,1-ethanediyl group, or a carbonyl group; Z⁵ represents a single bond, a methylene group, an ethylene group, a phenylene group, a fluorinated phenylene group, a phenylene group substituted with a trifluoromethyl group, —O—Z⁵¹—, —C(═O)—O—Z⁵¹—, or —C(═O)—NH—Z⁵¹—; Z⁵¹ represents an aliphatic hydrocarbylene group having 1 to 15 carbon atoms, a phenylene group, a fluorinated phenylene group, a phenylene group substituted with a trifluoromethyl group, or a combination thereof, Z⁵¹ optionally having a carbonyl group, an ester bond, an ether bond, a halogen atom, and/or a hydroxy group; R²¹ to R²⁸ each independently represent a halogen atom or a hydrocarbyl group having 1 to 25 carbon atoms and optionally having a heteroatom; R²³ and R²⁴ or R²⁶ and R²⁷ are optionally bonded to each other to form a ring together with the sulfur atom to which these groups are bonded; and M⁻ represents a non-nucleophilic counterion.

Such a repeating unit has a function as an acid generator in the base polymer.

The resist material preferably further comprises a surfactant.

Such a resist material can improve or regulate coatability of the resist material.

The present invention also provides a patterning process, comprising steps of: forming a resist film on a substrate by using the above resist material; exposing the resist film to high-energy ray; and developing the exposed resist film by using a developer.

Such a patterning process can form a good pattern.

KrF excimer laser light, ArF excimer laser light, electron beam, or extreme ultraviolet ray having a wavelength of 3 to 15 nm is preferably used as the high-energy ray.

Using such high energy ray can form a better pattern.

### Advantageous Effects of Invention

The sulfonium salt of the substituted or unsubstituted hydroxy(trifluoromethoxy)benzoic acid and/or the sulfonium salt of the substituted or unsubstituted hydroxy(trifluoromethylthio)benzoic acid is a quencher for inhibiting the acid diffusion. This quencher imparts low acid diffusion, and can improve LWR and CDU. These effects can construct a resist material having low LWR and improved CDU with high sensitivity.

### DESCRIPTION OF EMBODIMENTS

There have been demands for development of a quencher that can improve LWR of a line pattern or critical dimension uniformity (CDU) of a hole pattern, and that can also improve sensitivity in a resist material. For this performance, blur of an image due to the diffusion is required to be further reduced.

The present inventors have made earnest study to achieve the above object, and consequently found that a resist material in which a specific sulfonium salt is added can yield improved LWR and CDU, excellent resolution, and a wide process margin. The specific sulfonium salt, which is a quencher for inhibiting the acid diffusion, is a sulfonium salt of a substituted or unsubstituted hydroxy(di- or tri-fluoromethoxy)benzoic acid; a sulfonium salt of a substituted or unsubstituted hydroxy(di- or tri-fluoromethylthio)benzoic acid; a sulfonium salt of 2- or 3-, di- or tri-fluoromethoxybenzoic acid; and a sulfonium salt of 2- or 3-, di- or tri-fluoromethylthiobenzoic acid. The anion having the substituted or unsubstituted hydroxy group and carboxy group in the molecule of the anion yields an excellent effect of inhibiting the acid diffusion, and when the hydroxy group is substituted with an acid-labile group, dissolution contrast is improved. The anion having the trifluoromethoxy group or trifluoromethylthio group yields a high electron-withdrawing effect and an effect of preventing aggregation of the quencher with electron repulsion of the balky group. This finding has led to complete the present invention.

Specifically, the present invention is a resist material comprising, as a quencher: a sulfonium salt of a substituted or unsubstituted hydroxy(trifluoromethoxy)benzoic acid; and/or a sulfonium salt of a substituted or unsubstituted hydroxy(trifluoromethylthio)benzoic acid.

In addition, the present invention is a resist material comprising, as a quencher, one or more of: a sulfonium salt of a substituted or unsubstituted hydroxy(difluoromethoxy)benzoic acid; a sulfonium salt of a substituted or unsubstituted hydroxy(difluoromethylthio)benzoic acid; a sulfonium salt of a 2- or 3-, di- or tri-fluoromethoxybenzoic acid; and a sulfonium salt of a 2- or 3-, di- or tri-fluoromethylthiobenzoic acid.

Hereinafter, the present invention will be described in detail, but the present invention is not limited thereto.

Resist Material

The inventive resist material comprises, as a quencher, a sulfonium salt of a substituted or unsubstituted hydroxy(trifluoromethoxy)benzoic acid; and/or a sulfonium salt of a substituted or unsubstituted hydroxy(trifluoromethylthio)benzoic acid, or one or more of: a sulfonium salt of a substituted or unsubstituted hydroxy(difluoromethoxy)benzoic acid; a sulfonium salt of a substituted or unsubstituted hydroxy(difluoromethylthio)benzoic acid; a sulfonium salt of a 2- or 3-, di- or tri-fluoromethoxybenzoic acid; and a sulfonium salt of a 2- or 3-, di- or tri-fluoromethylthiobenzoic acid.

Sulfonium Salt of Substituted or Unsubstituted Hydroxy(di or trifluoromethoxy)benzoic Acid; Sulfonium Salt of Substituted or Unsubstituted Hydroxy(di or trifluoromethylthio)benzoic acid; Sulfonium Salt of 2- or 3-, di- or tri-fluoromethoxybenzoic Acid; and Sulfonium Salt of 2- or 3-, di- or tri-fluoromethylthiobenzoic Acid.

One or more of the sulfonium salt of the substituted or unsubstituted hydroxy(trifluoromethoxy)benzoic acid and/or the sulfonium salt of the substituted or unsubstituted hydroxy(trifluoromethylthio)benzoic acid; and the sulfonium salt of the substituted or unsubstituted hydroxy(difluoromethoxy)benzoic acid, the sulfonium salt of the substituted or unsubstituted hydroxy(difluoromethylthio)benzoic acid, the sulfonium salt of the 2- or 3-, di- or tri-fluoromethoxybenzoic acid, and the sulfonium salt of the 2- or 3-, di- or tri-fluoromethylthiobenzoic acid (hereinafter, also referred to as “sulfonium salt A”) are preferably represented by the following general formula (1) and the following general formula (1′), respectively.

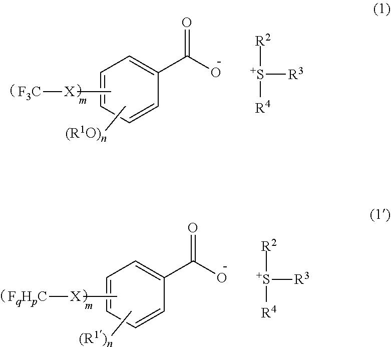

In the general formula (1), R¹ represents a hydrogen atom or a group selected from a linear, branched, or cyclic alkyl group, alkoxy group, alkoxycarbonyl group, or acyl group having 1 to 15 carbon atoms, an alkenyl group having 2 to 15 carbon atoms, and an alkynyl group having 2 to 15 carbon atoms, the group optionally having a halogen atom, a carbonyl group, an ether bond, a substituted or unsubstituted aryl group, or a hydroxy group, and the group being optionally an acid-labile group. X represents an oxygen atom or a sulfur atom. “m” and “n” each represent 1 or 2. R² to R⁴ each independently represent a halogen atom or a hydrocarbyl group having 1 to 25 carbon atoms and optionally having a heteroatom. R² and R³ are optionally bonded to each other to form a ring together with the sulfur atom to which these groups are bonded.

In the general formula (1′), R^{1′} represents a hydrogen atom, a halogen atom, or a group selected from a hydroxy group, a substituted or unsubstituted amino group, a linear, branched, or cyclic alkyl group, alkoxy group, alkoxycarbonyl group, alkoxycarbonyloxy group, acyl group, or acyloxy group having 1 to 15 carbon atoms, an alkenyloxy group having 2 to 15 carbon atoms, and an alkynyloxy group having 2 to 15 carbon atoms, the group optionally having a halogen atom, a carbonyl group, an ether bond, a substituted or unsubstituted aryl group, or a hydroxy group, the group being optionally an acid-labile group, and when R^{1′} represents a hydrogen atom, the substituting position of the F_{q}HₚC—X— group is the 2- or 3-position. “p” represents 0 or 1, and “q” represents 2 or 3. X represents an oxygen atom or a sulfur atom. “m” and “n” each represent 1 or 2. R² to R⁴ each independently represent a halogen atom or a hydrocarbyl group having 1 to 25 carbon atoms and optionally having a heteroatom. R² and R³ are optionally bonded to each other to form a ring together with the sulfur atom to which these groups are bonded.

Examples of the anion of the sulfonium salt represented by the general formula (1) include the following anions, but the anion is not limited thereto.

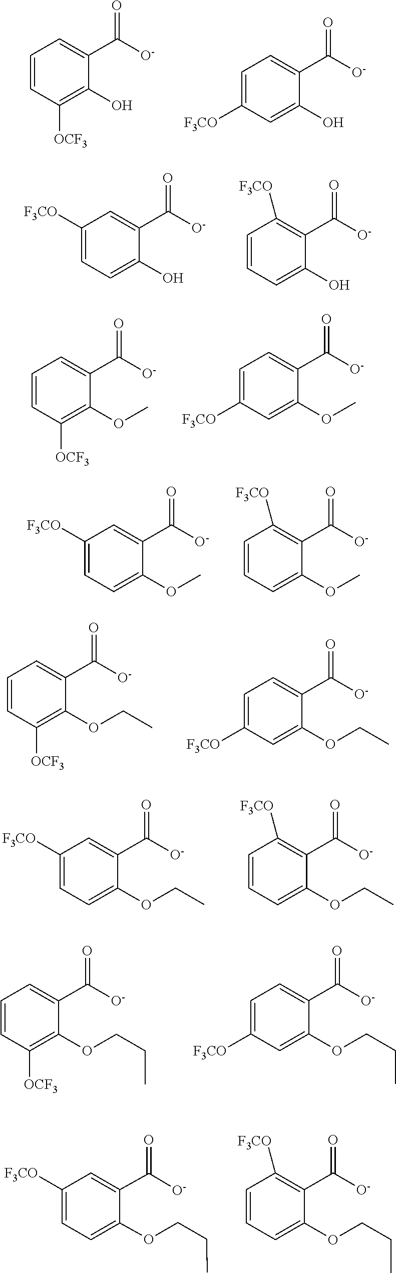

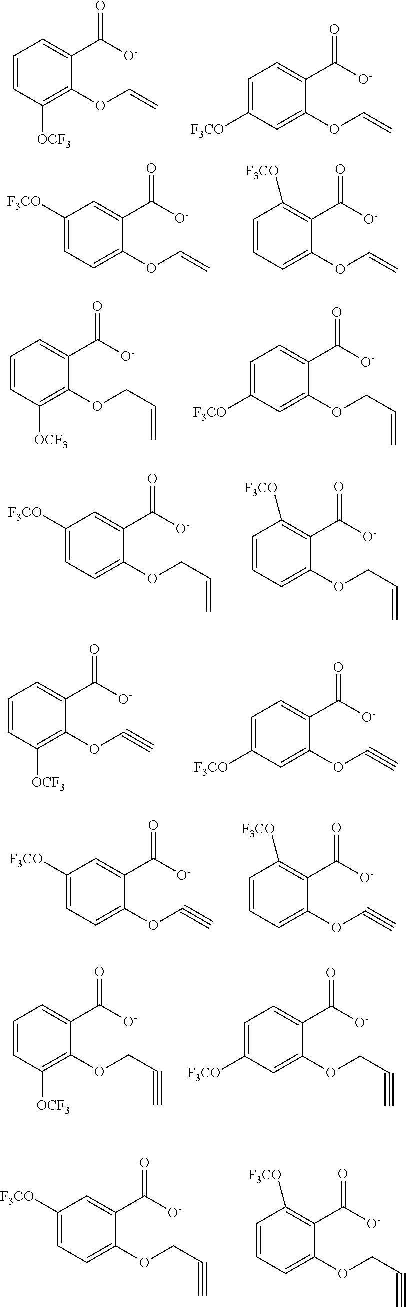

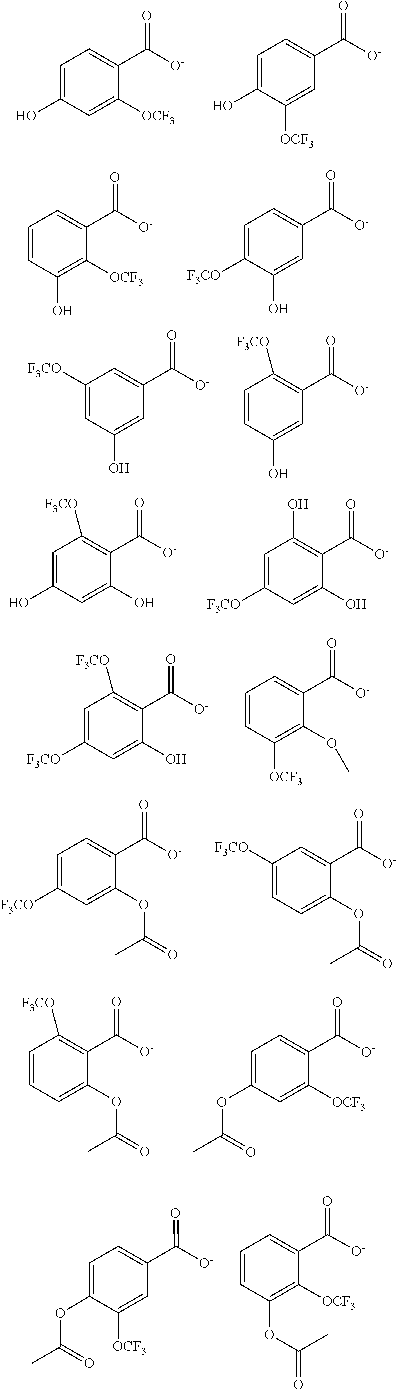

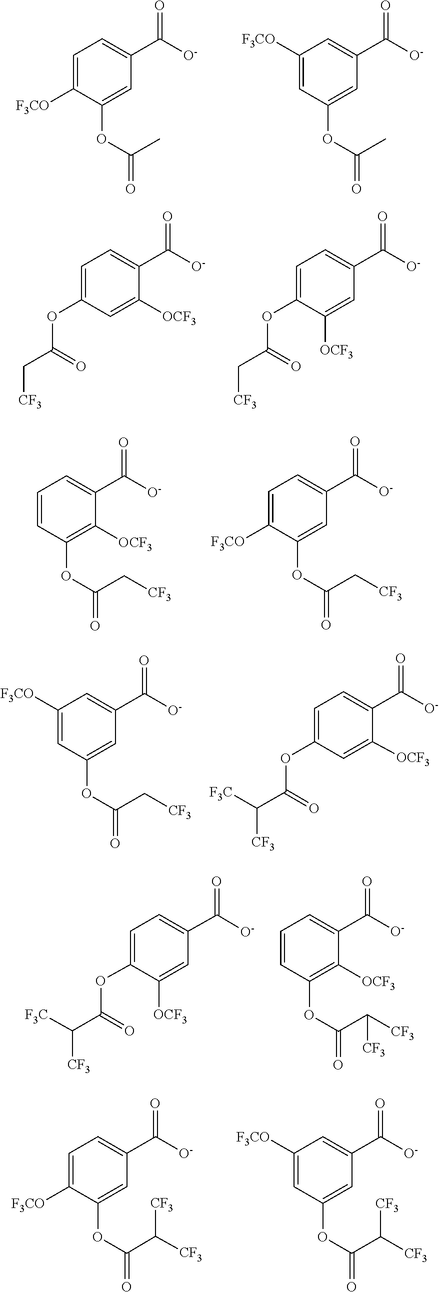

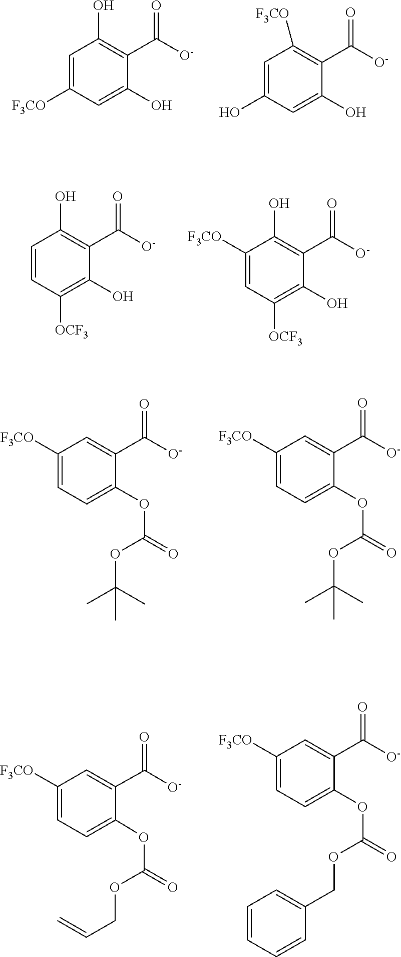

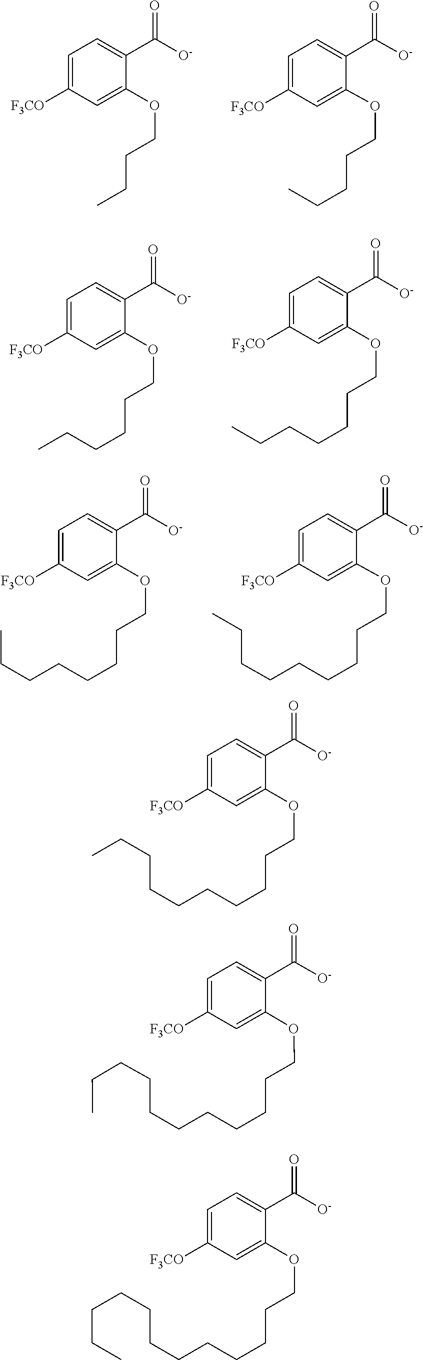

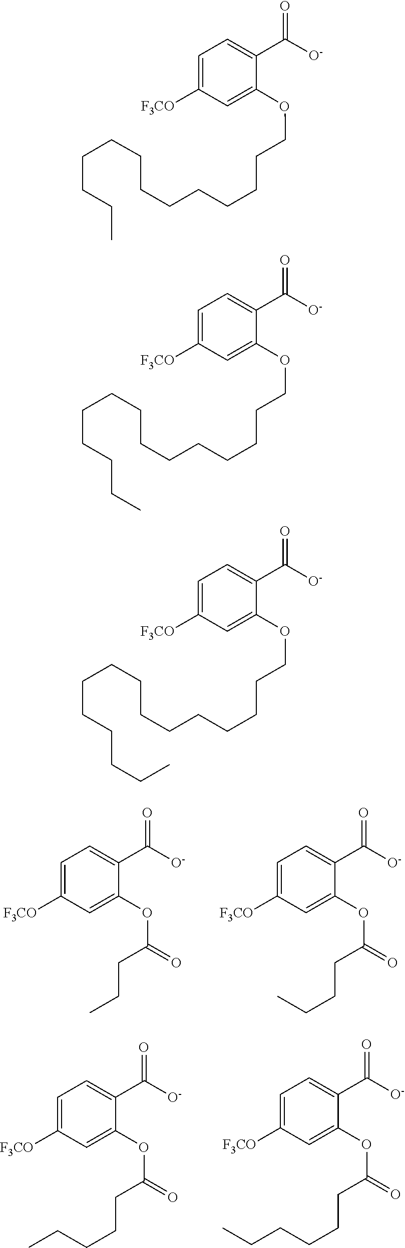

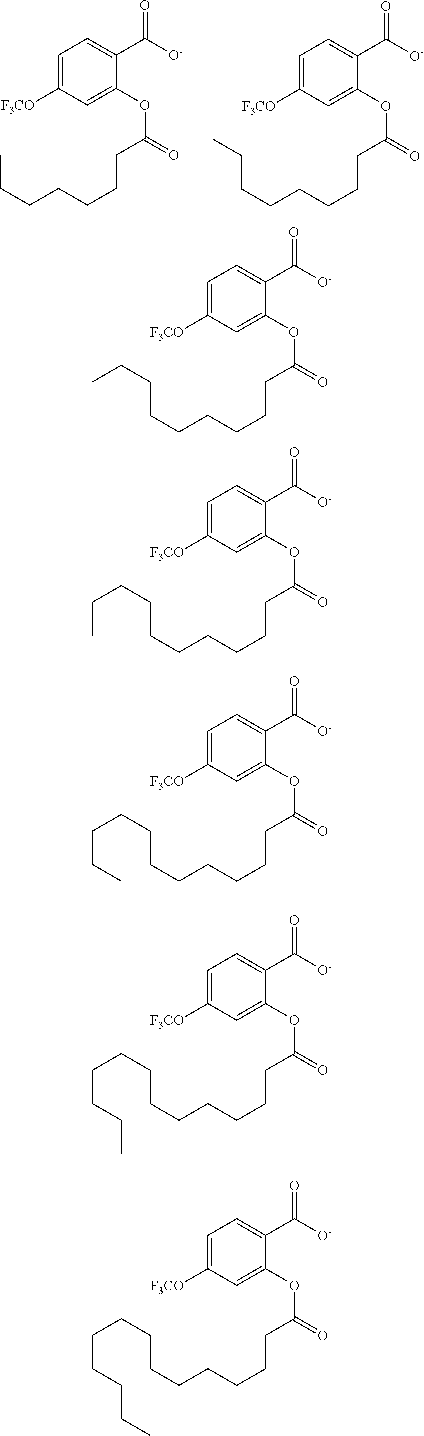

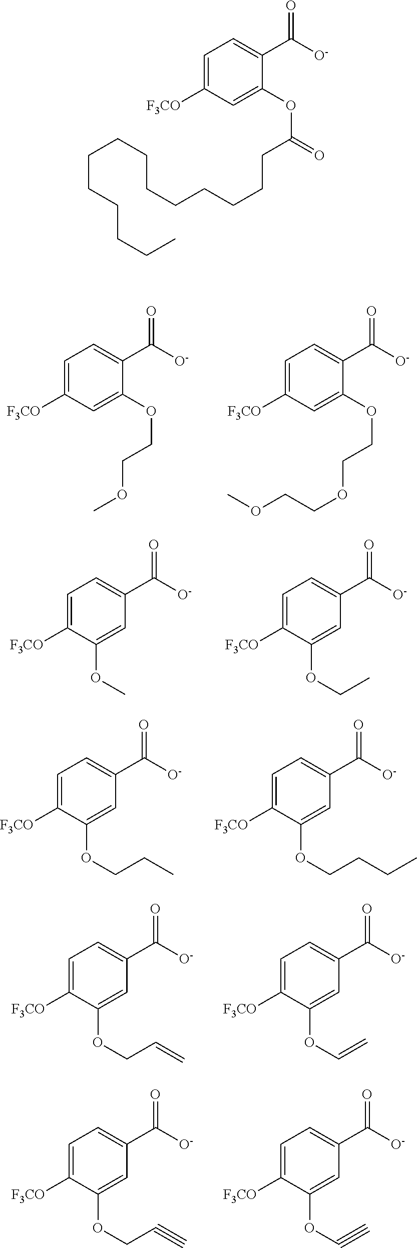

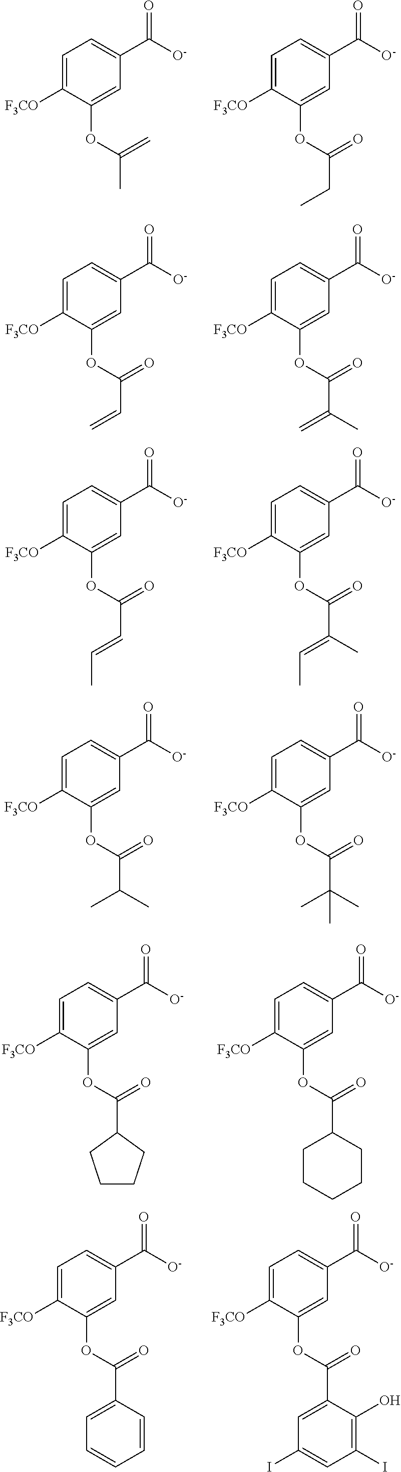

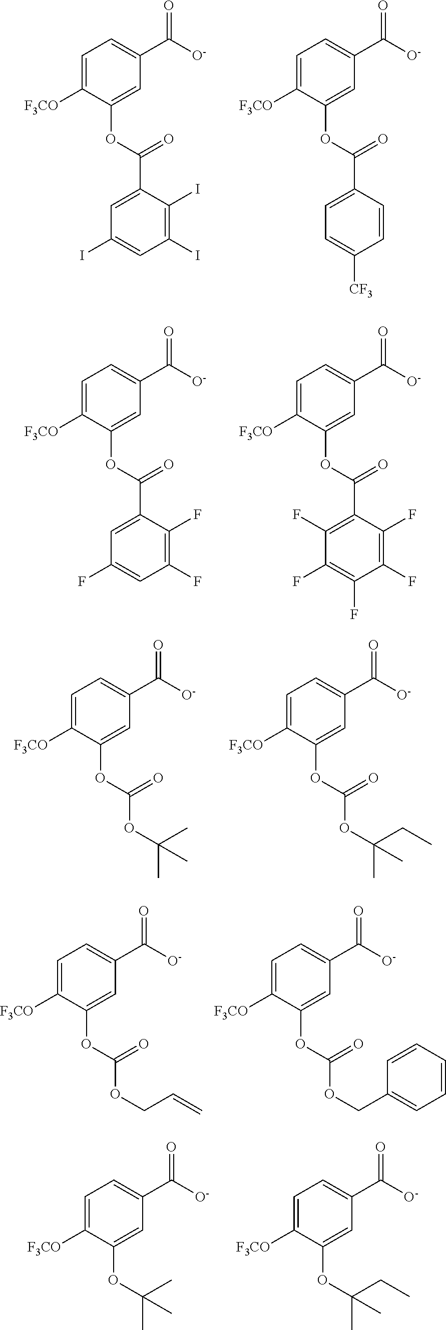

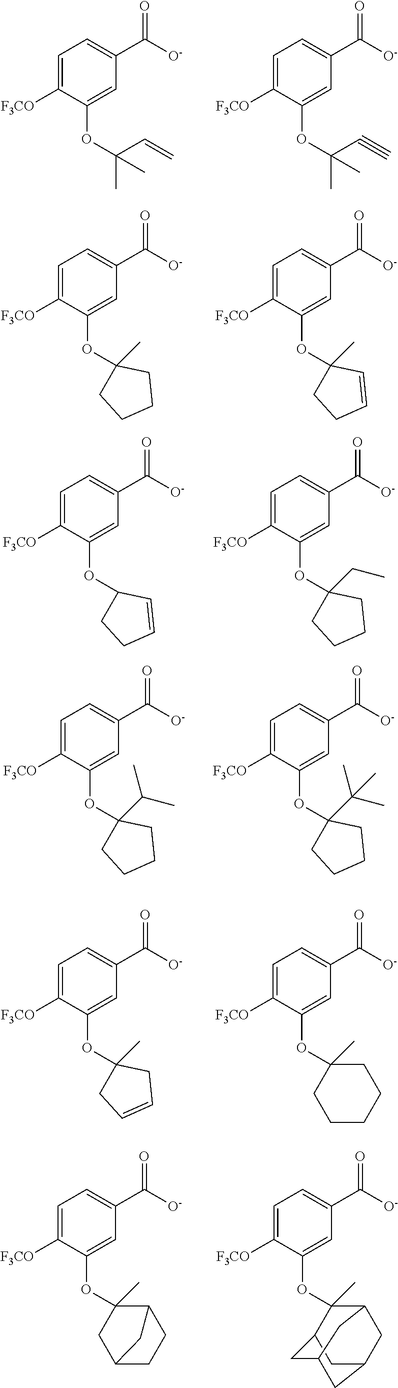

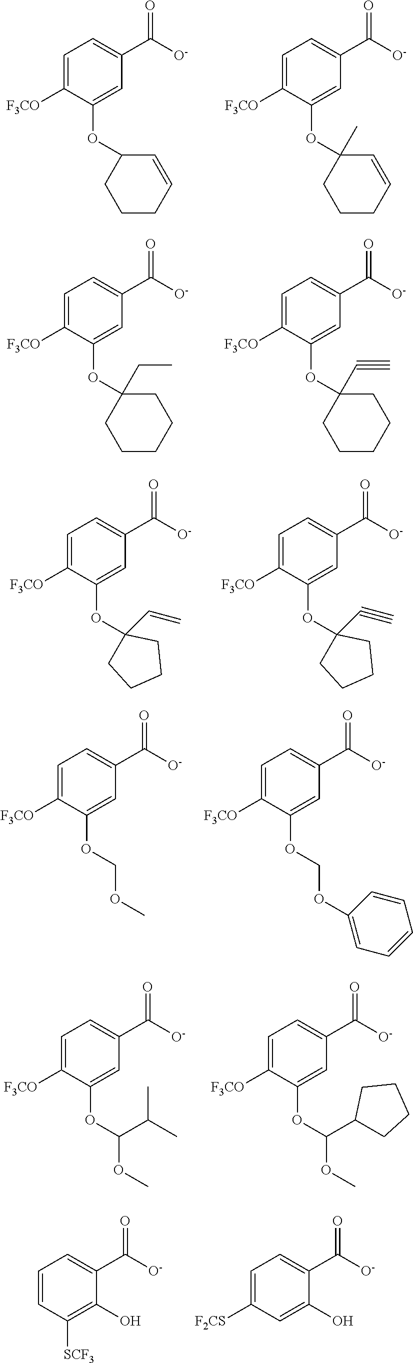

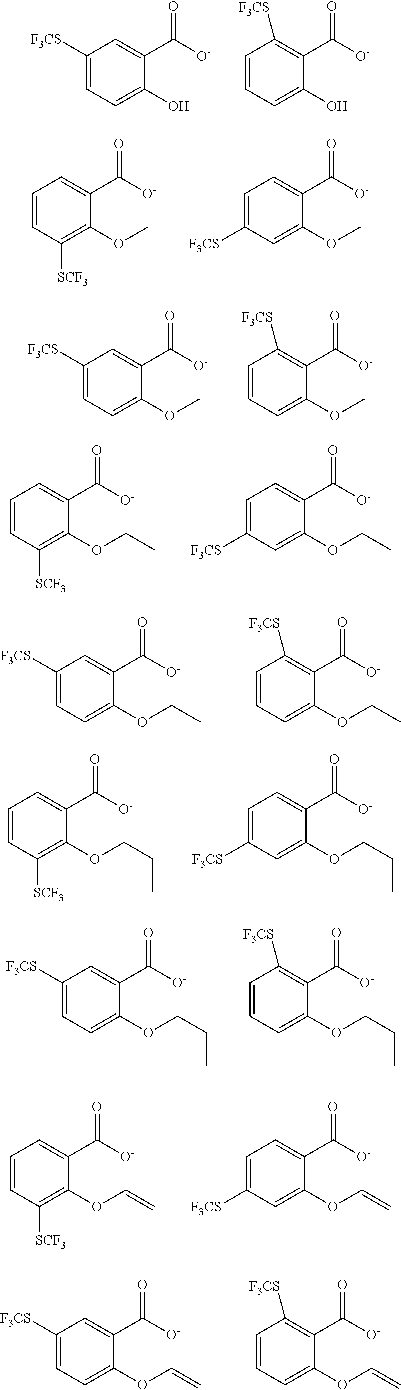

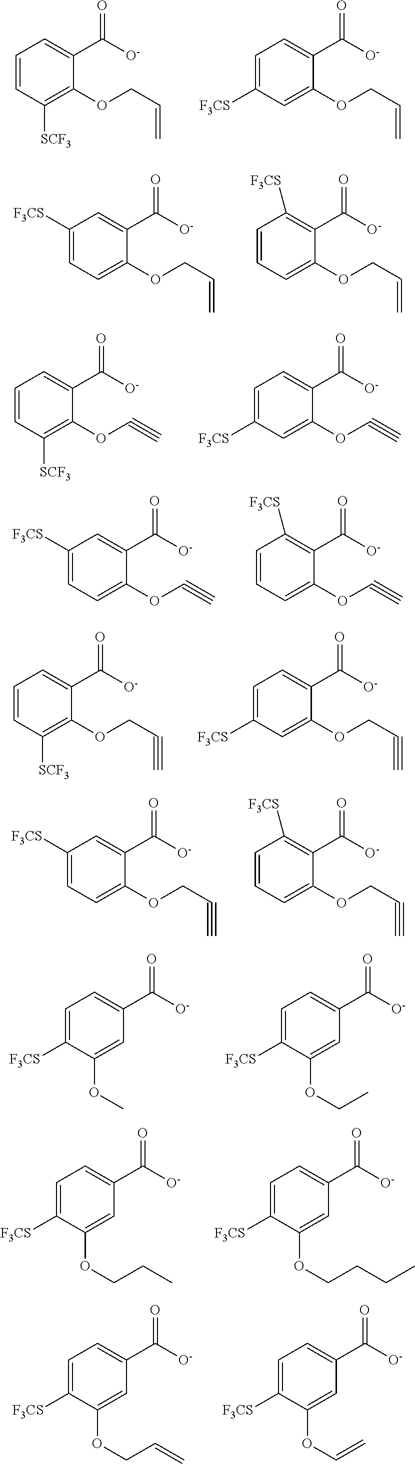

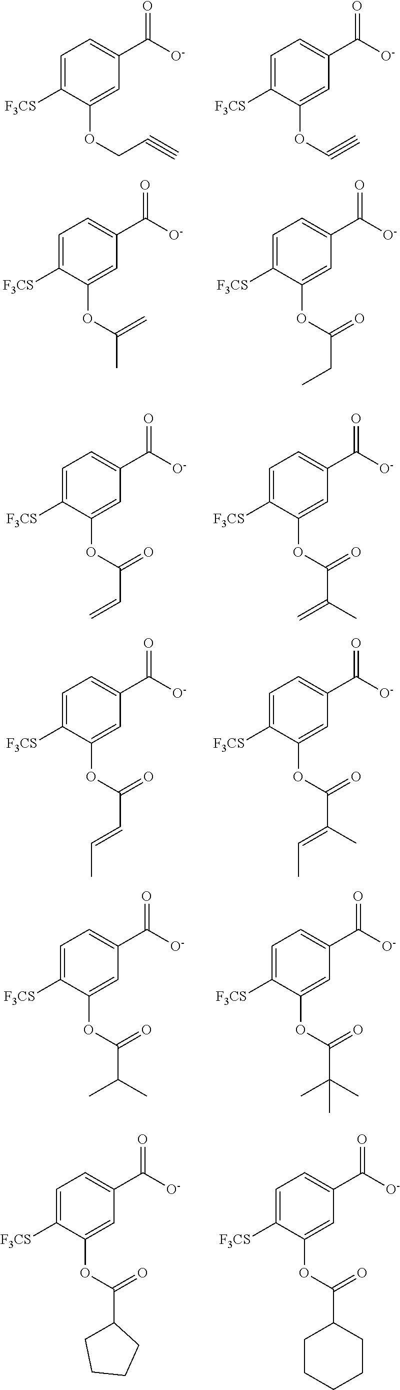

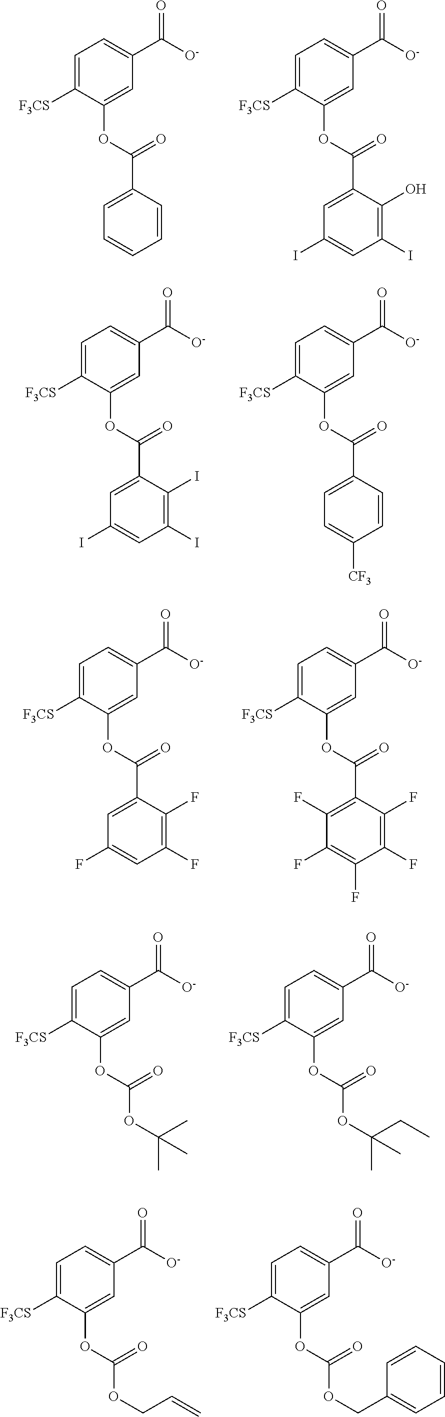

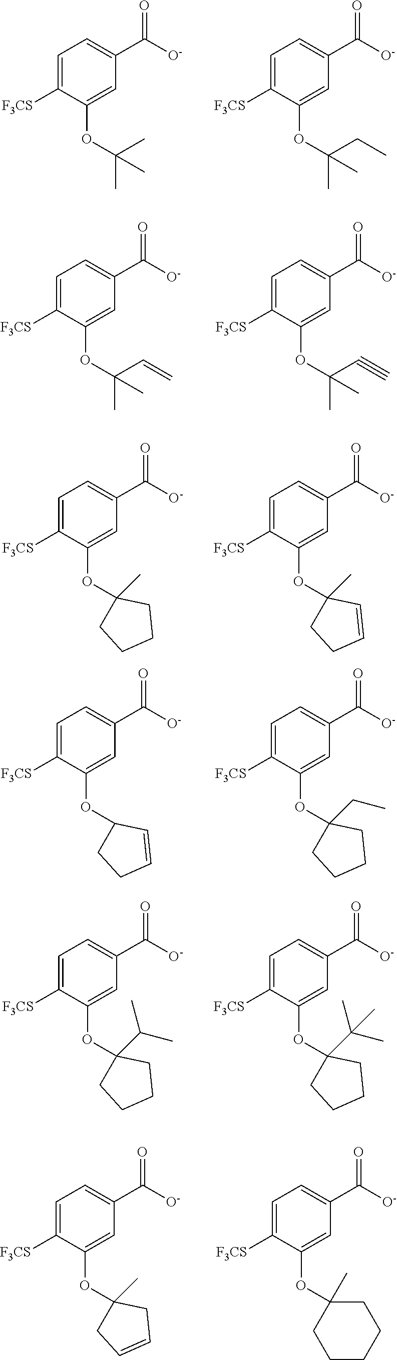

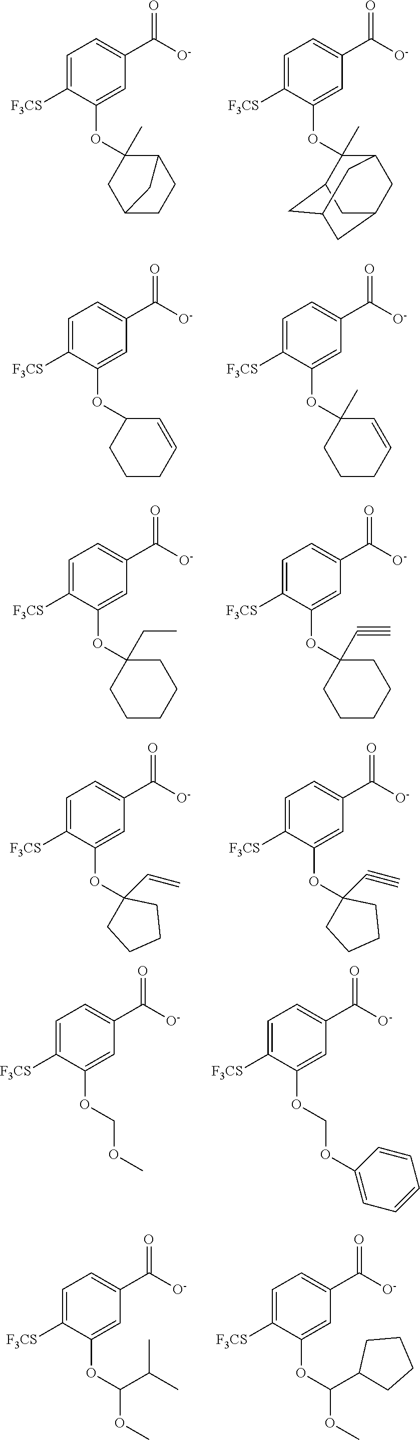

Examples of the anion of the sulfonium salt represented by the general formula (1′) include: those exemplified as the specific examples of the anion of the sulfonium salt represented by the general formula (1); and the following anions, but the anion is not limited thereto.

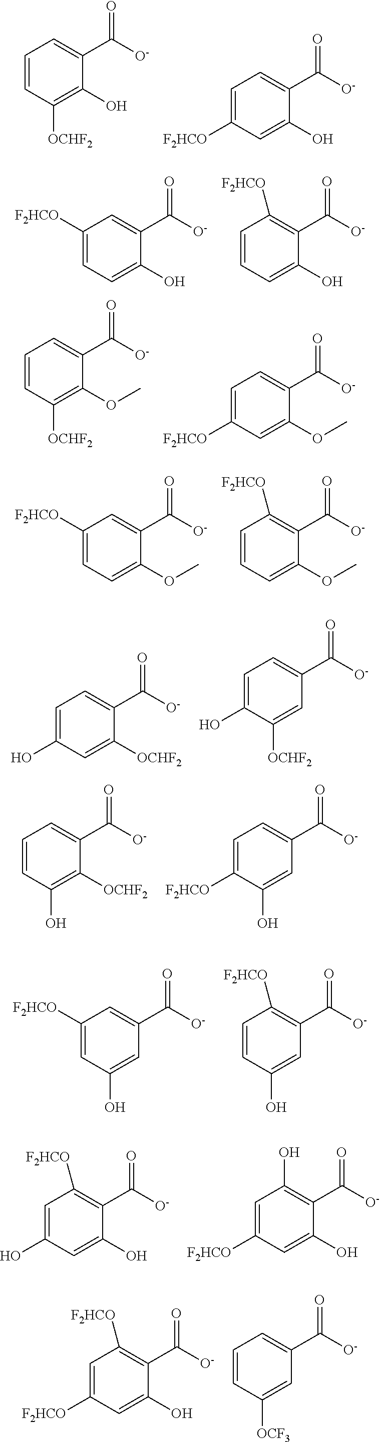

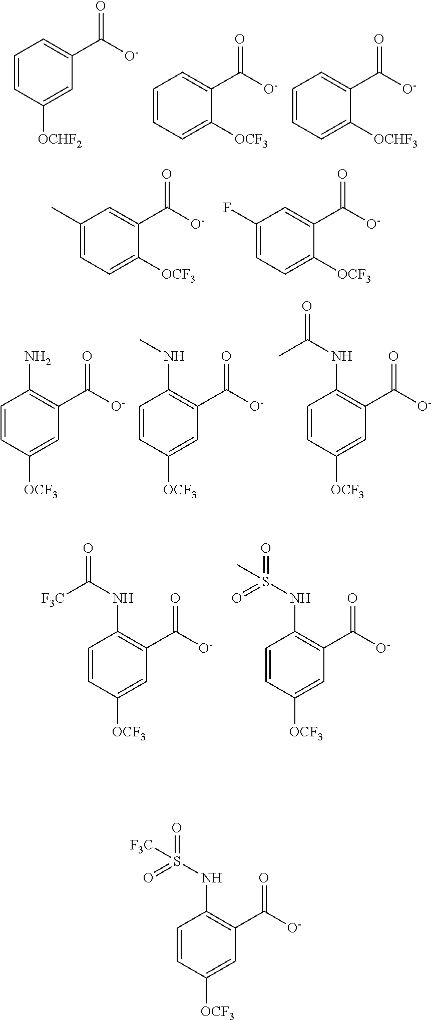

In the general formulae (1) and (1′), R² to R⁴ each independently represent a halogen atom or a hydrocarbyl group having 1 to 25, preferably 1 to 20, carbon atoms and optionally having a heteroatom.

Examples of the halogen atom represented by R² to R⁴ include a fluorine atom, a chlorine atom, a bromine atom, and an iodine atom.

The hydrocarbyl group represented by R² to R⁴ and having 1 to 25 carbon atoms may be a saturated or unsaturated group, and may be any of linear, branched, and cyclic groups. Specific examples thereof include: alkyl groups having 1 to 25 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-octyl group, an n-nonyl group, an n-decyl group, an undecyl group, a dodecyl group, a tridecyl group, a tetradecyl group, a pentadecyl group, a heptadecyl group, an octadecyl group, a nonadecyl group, and an icosyl group; cyclic saturated hydrocarbyl groups having 3 to 25 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 25 carbon atoms, such as a vinyl group, a propenyl group, a butenyl group, and a hexenyl group; alkynyl groups having 2 to 25 carbon atoms, such as an ethynyl group, a propynyl group, and a butynyl group; cyclic unsaturated aliphatic hydrocarbyl groups having 3 to 25 carbon atoms, such as a cyclohexenyl group and a norbornenyl group; aryl groups having 6 to 25 carbon atoms, such as a phenyl group, a methylphenyl group, an ethylphenyl group, an n-propylphenyl group, an isopropylphenyl group, an n-butylphenyl group, an isobutylphenyl group, a sec-butylphenyl group, a tert-butylphenyl group, a naphthyl group, a methylnaphthyl group, an ethylnaphthyl group, an n-propylnaphthyl group, an isopropylnaphthyl group, an n-butylnaphthyl group, an isobutylnaphthyl group, a sec-butylnaphthyl group, and a tert-butylnaphthyl group; aralkyl groups having 7 to 25 carbon atoms, such as a benzyl group and a phenethyl group; and groups obtained by combining these groups.

A part or all of hydrogen atoms in the hydrocarbyl group are optionally substituted with a group having a heteroatom such as an oxygen atom, a sulfur atom, a nitrogen atom, and a halogen atom. A part of —CH₂— in the hydrocarbyl group is optionally substituted with a group having a heteroatom such as an oxygen atom, a sulfur atom, and a nitrogen atom. As a result, optionally contained are a hydroxy group, a fluorine atom, a chlorine atom, a bromine atom, an iodine atom, a cyano group, a nitro group, a mercapto group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

R² and R³ are optionally bonded to each other to form a ring together with the sulfur atom to which these groups are bonded. In this case, the ring preferably has the following structures.

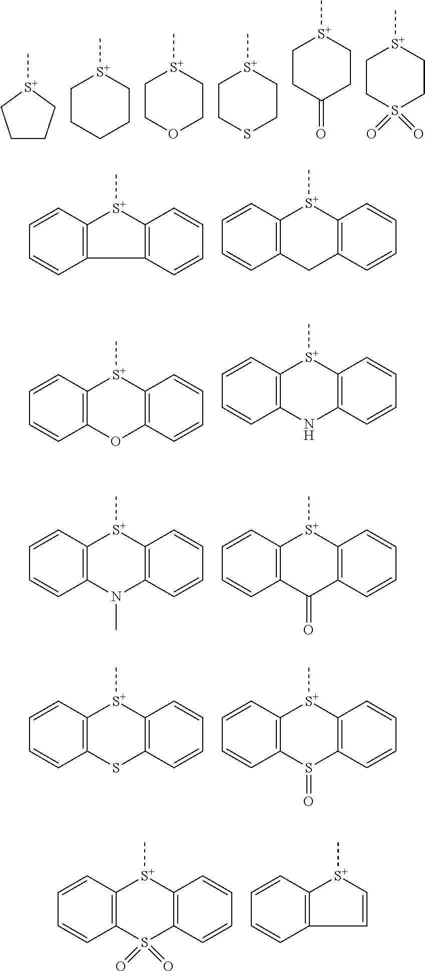

In the formulae, a broken line represents an attachment point to R⁴.

Examples of the cation of the sulfonium salt represented by the general formulae (1) and (1′) include the following cations, but the cation is not limited thereto.

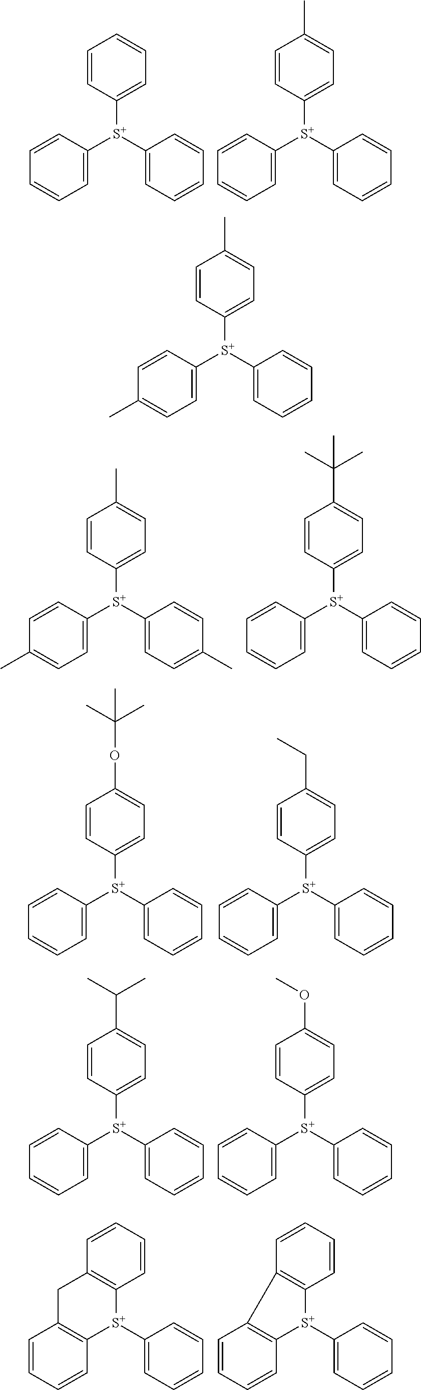

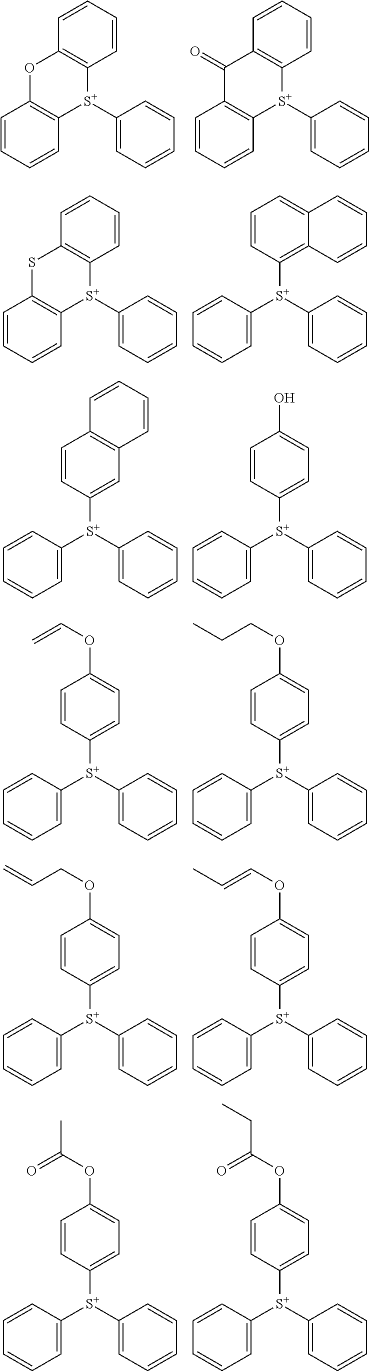

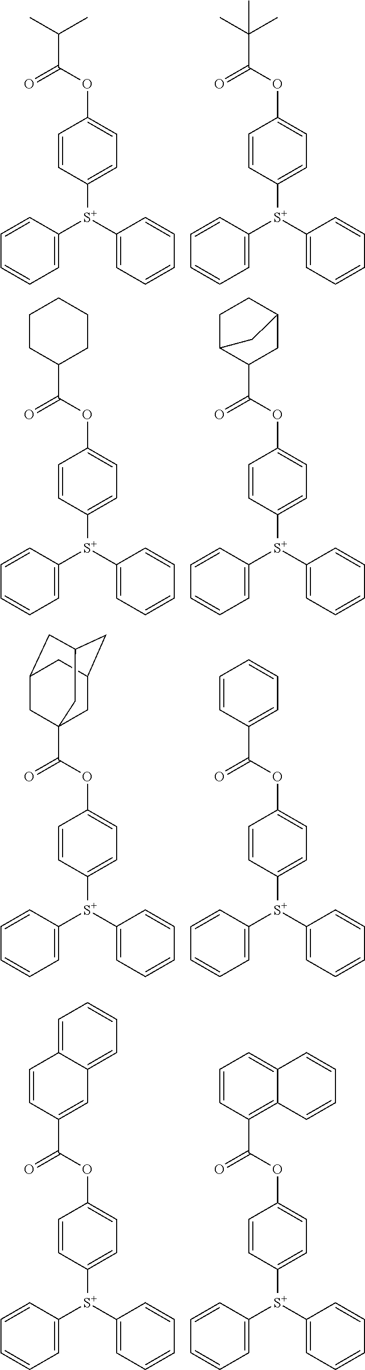

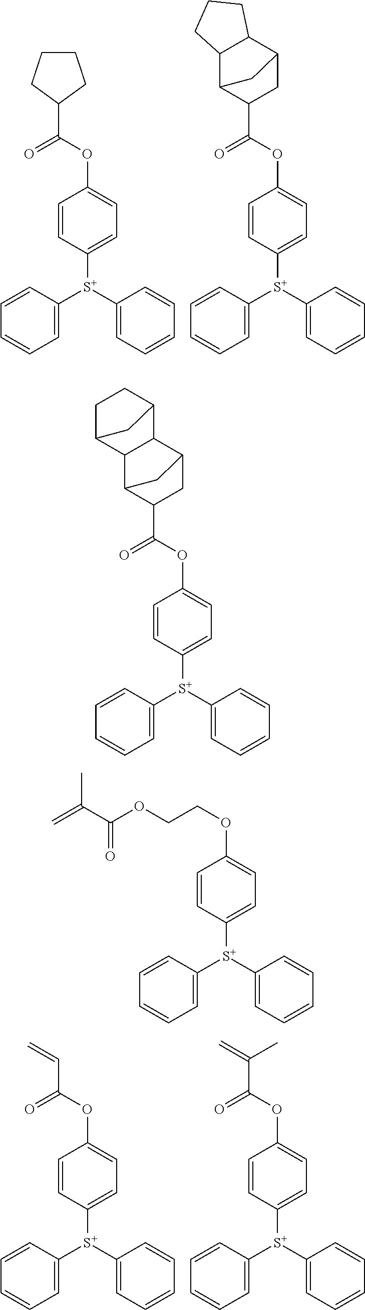

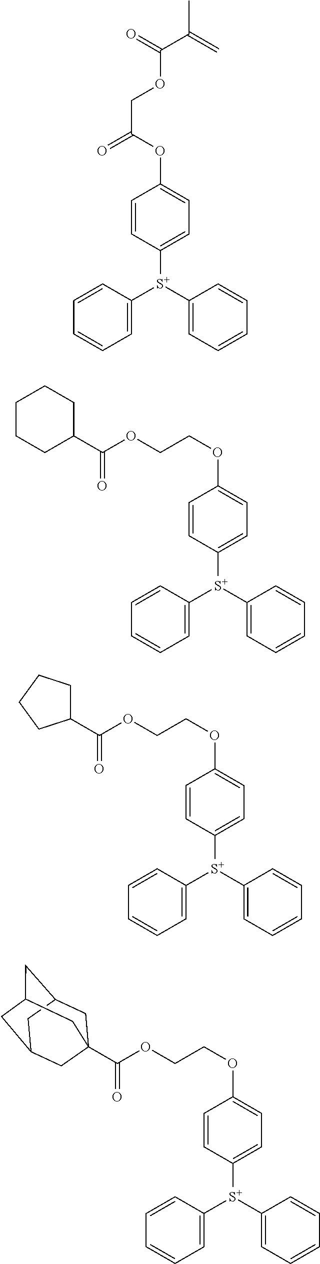

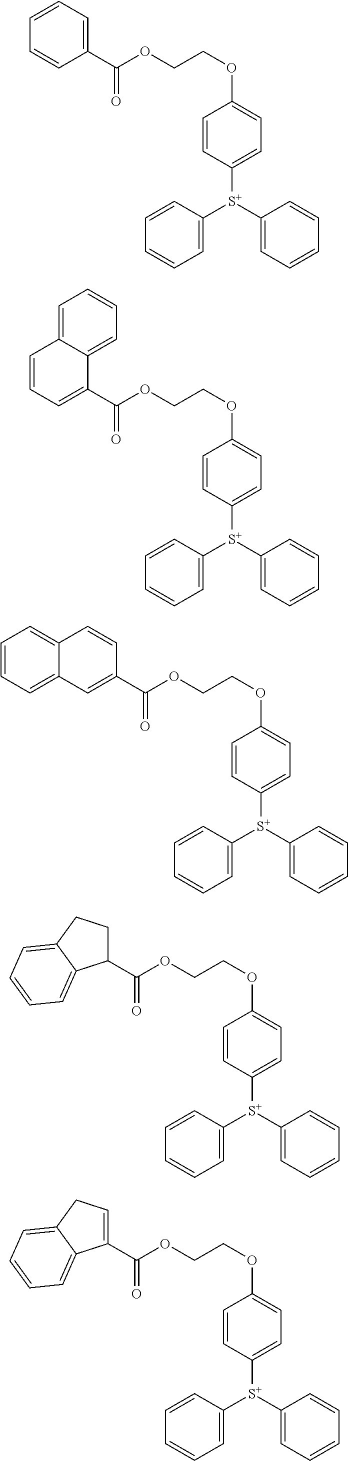

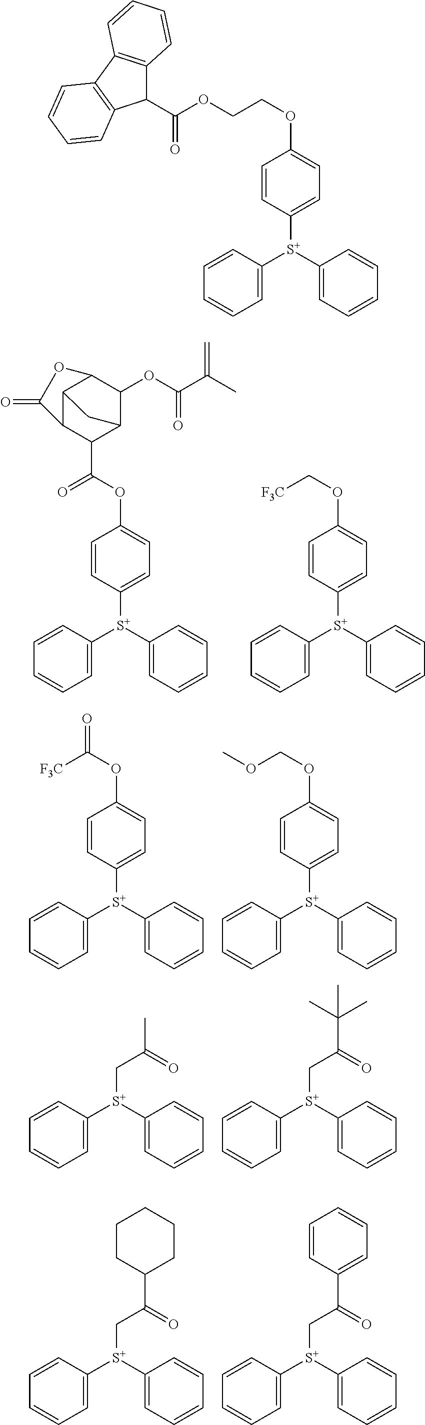

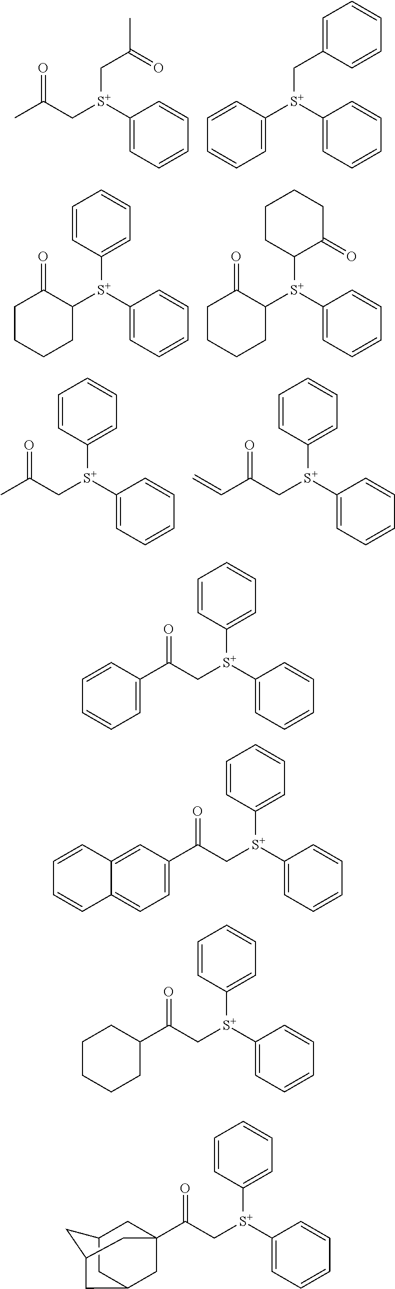

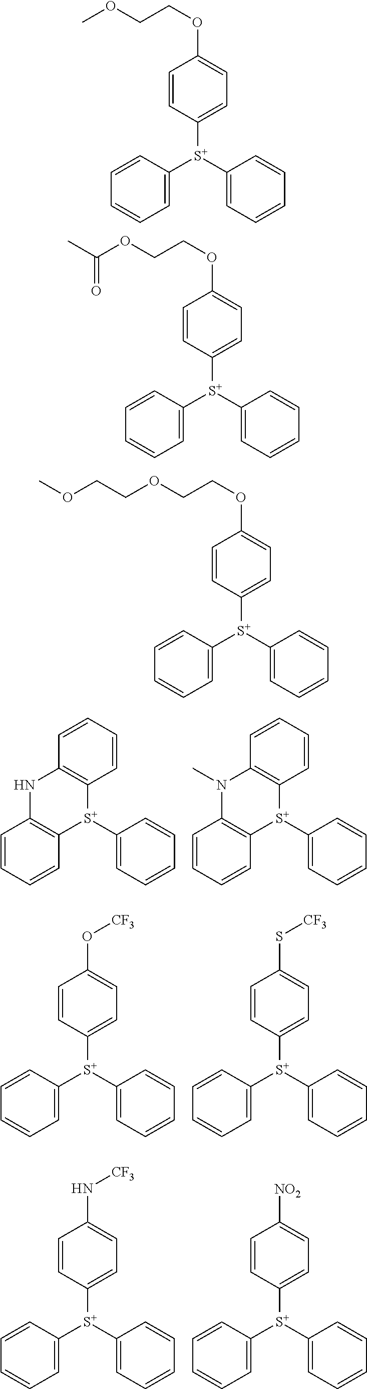

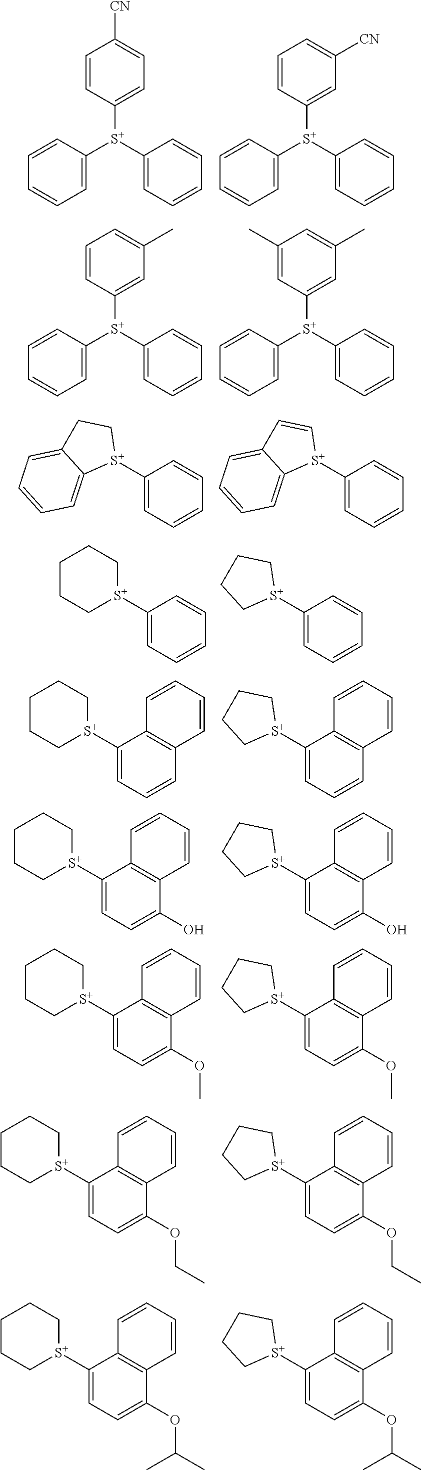

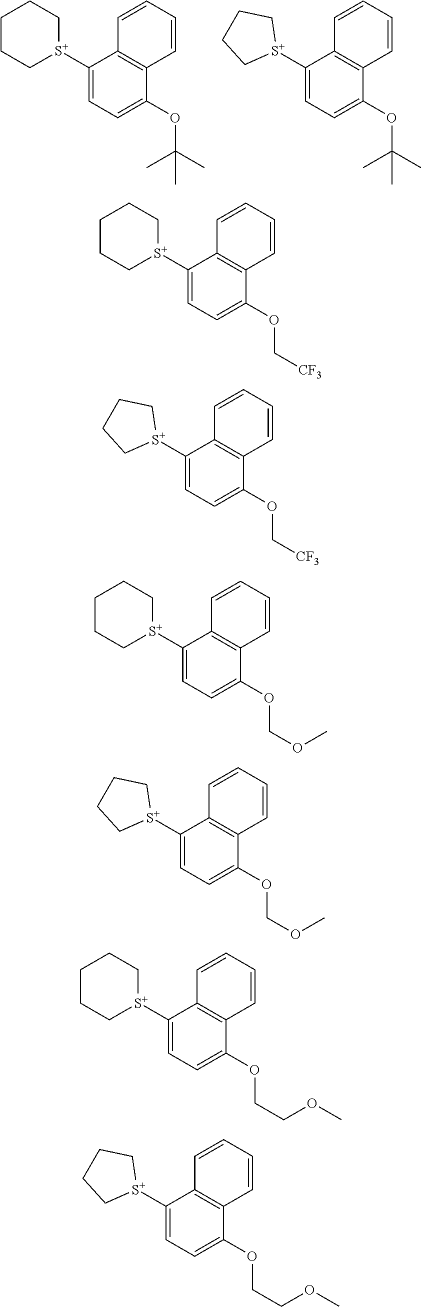

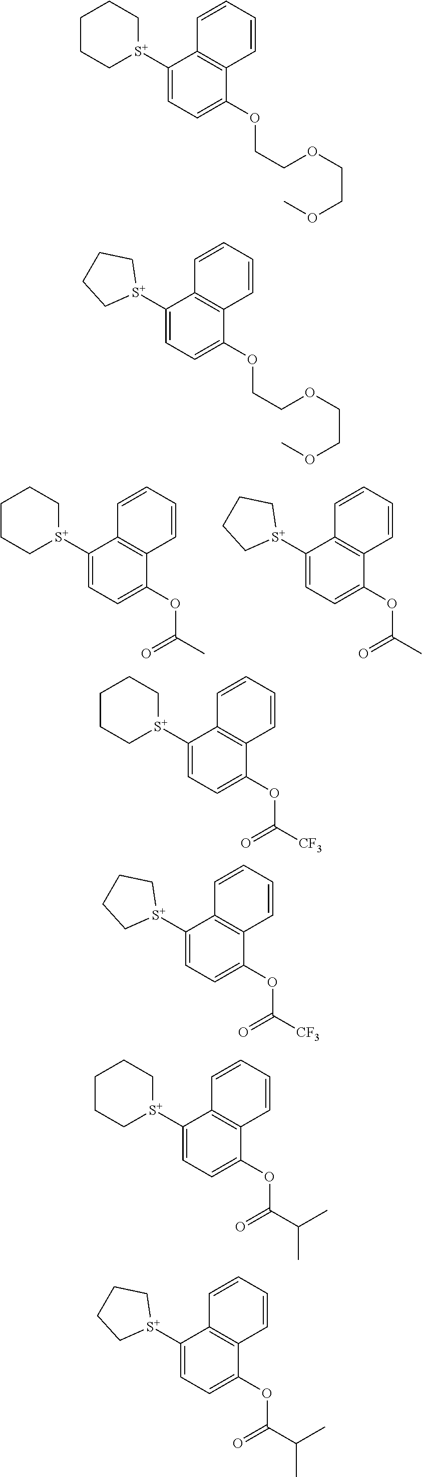

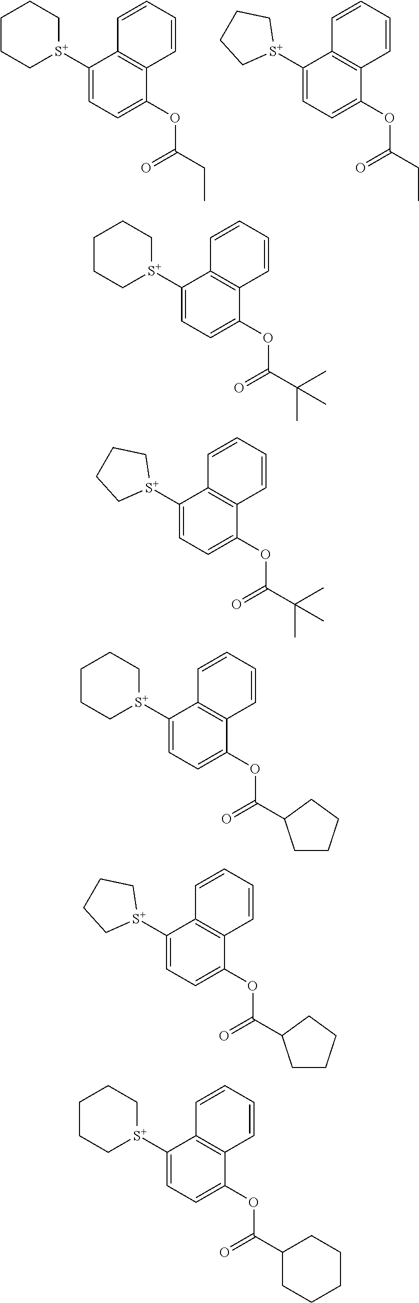

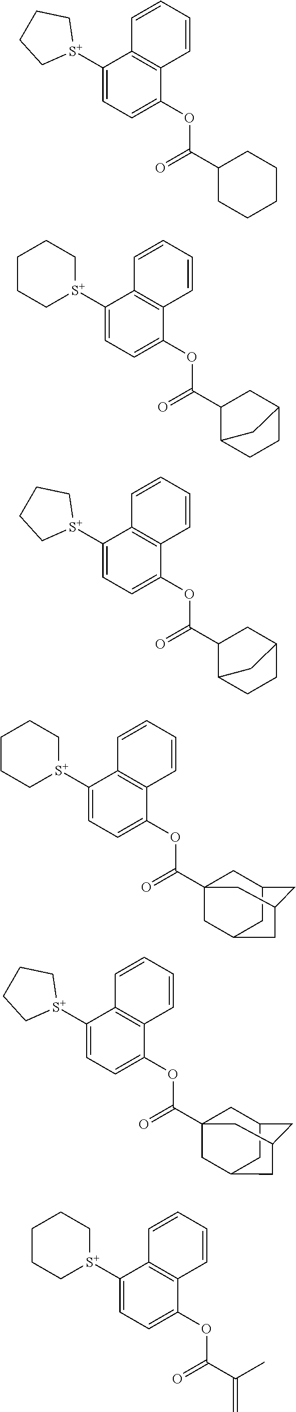

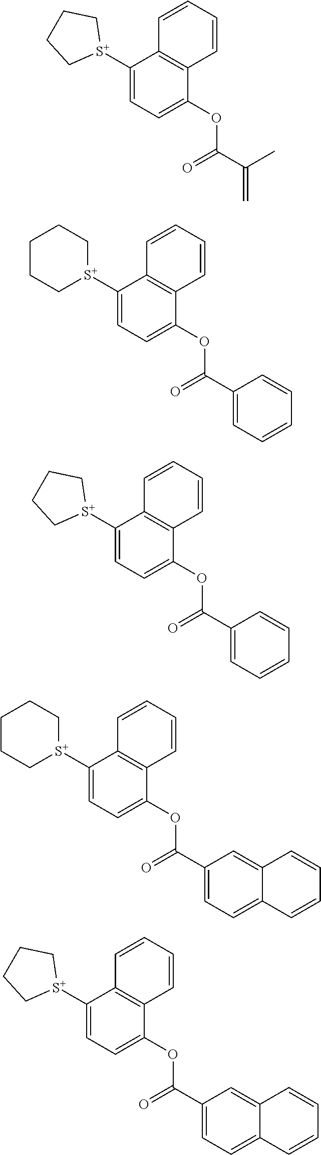

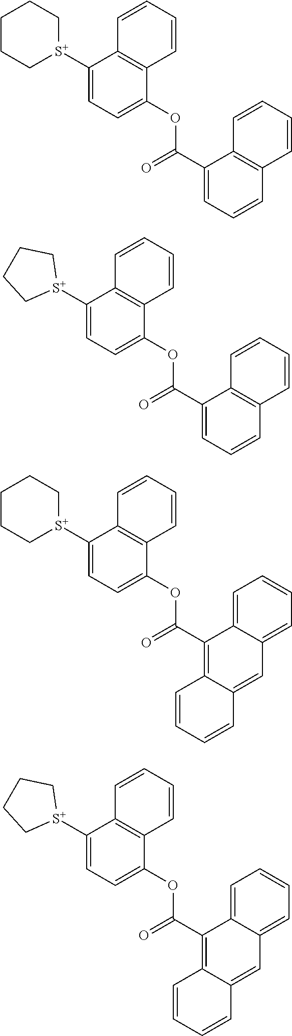

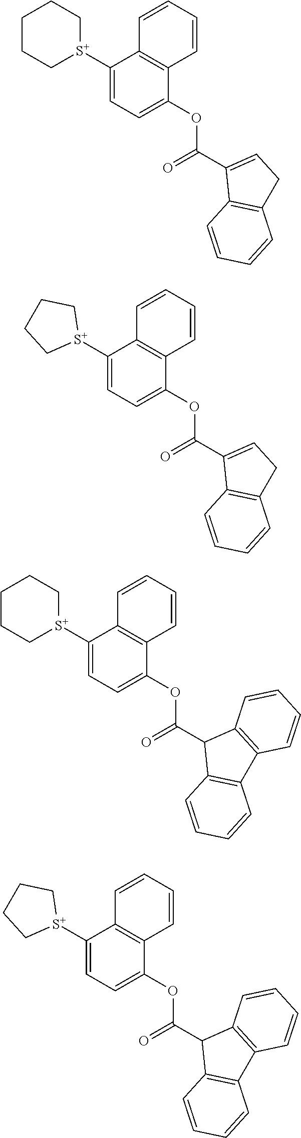

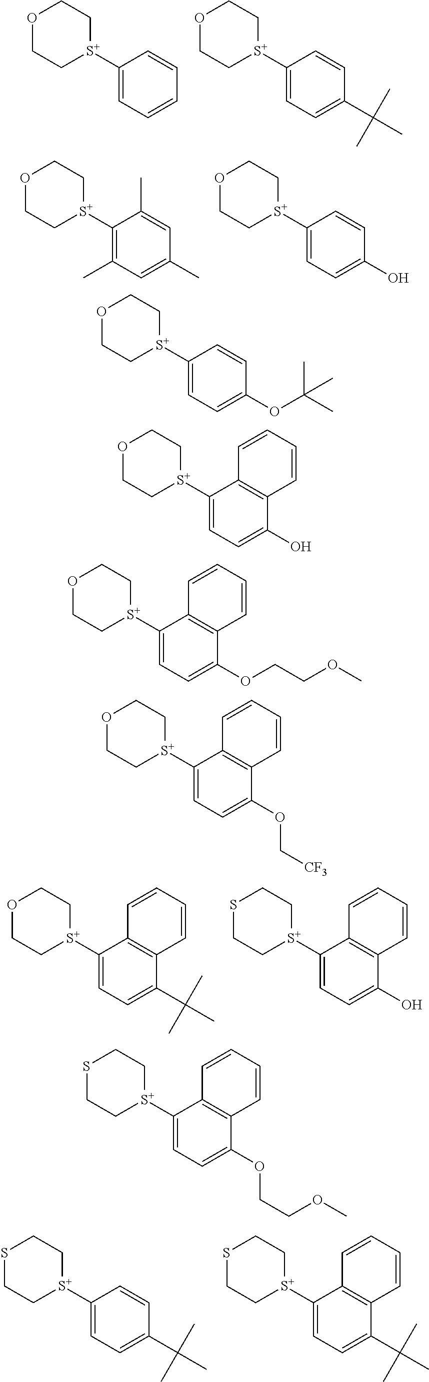

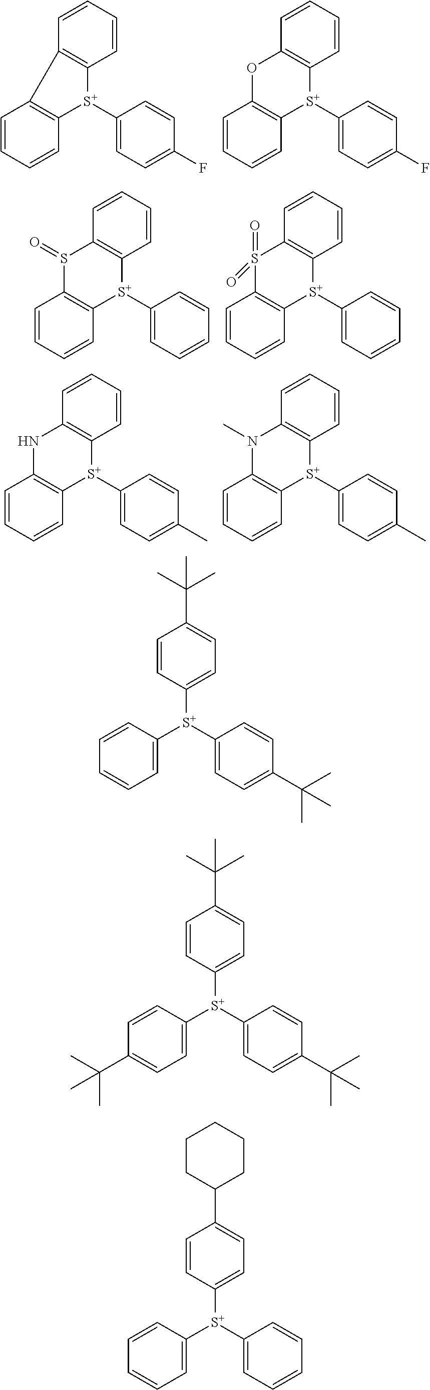

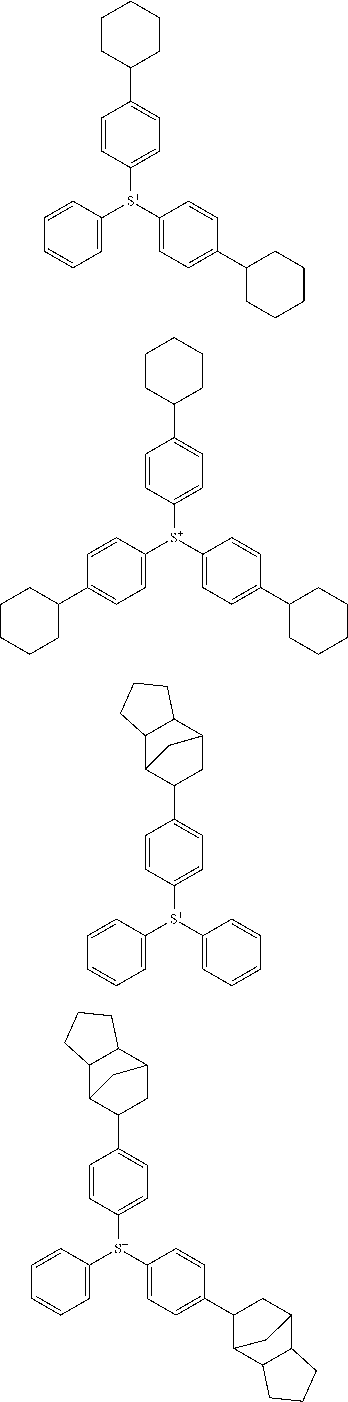

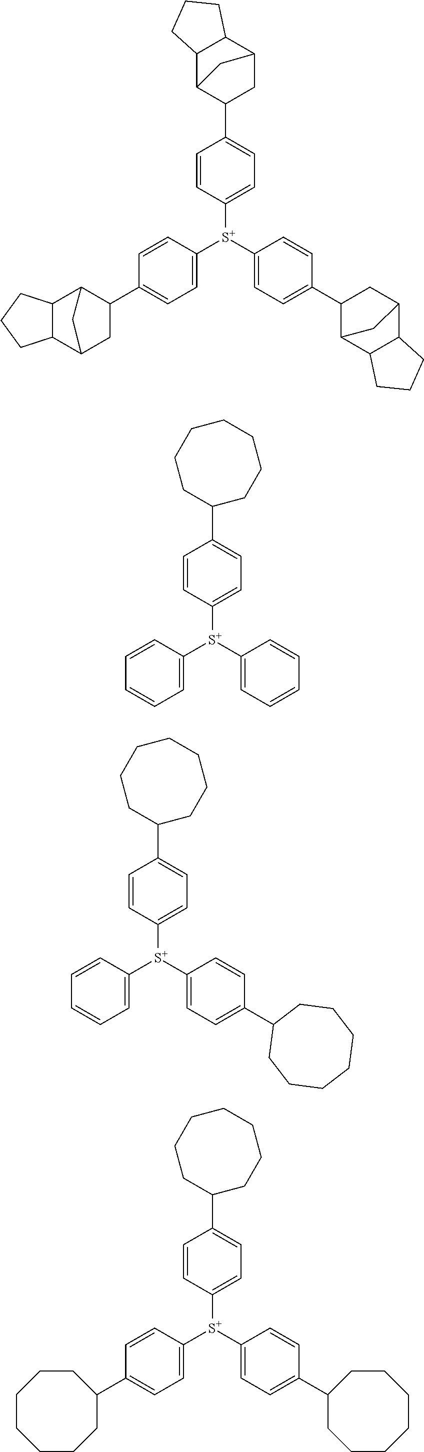

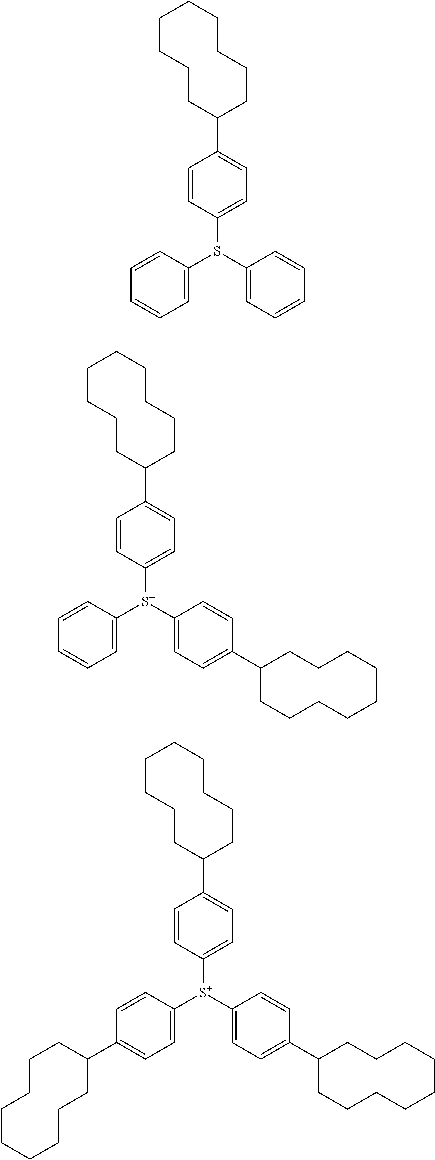

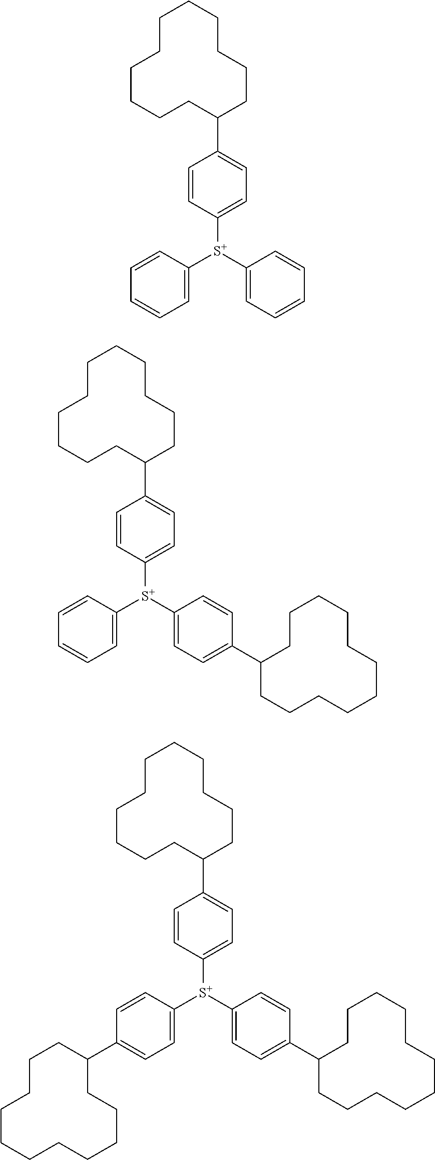

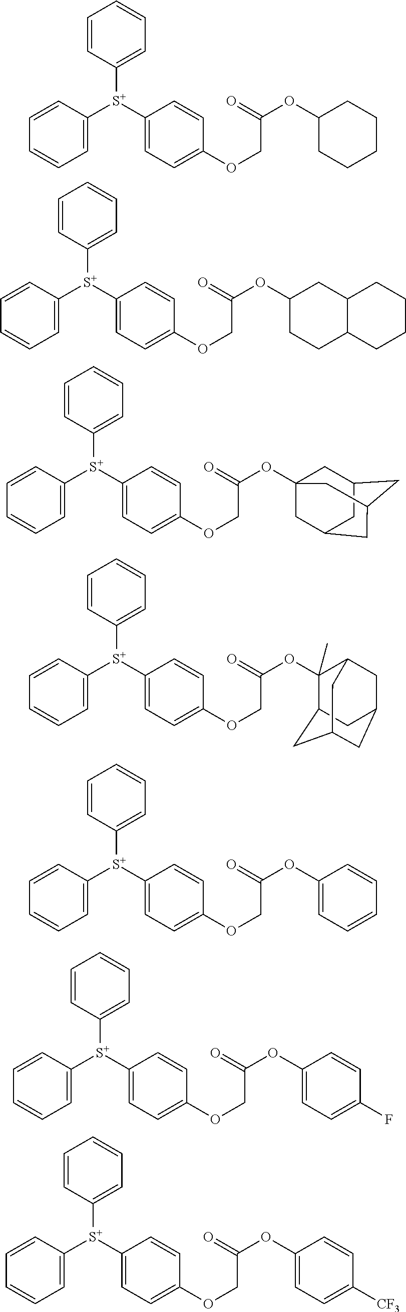

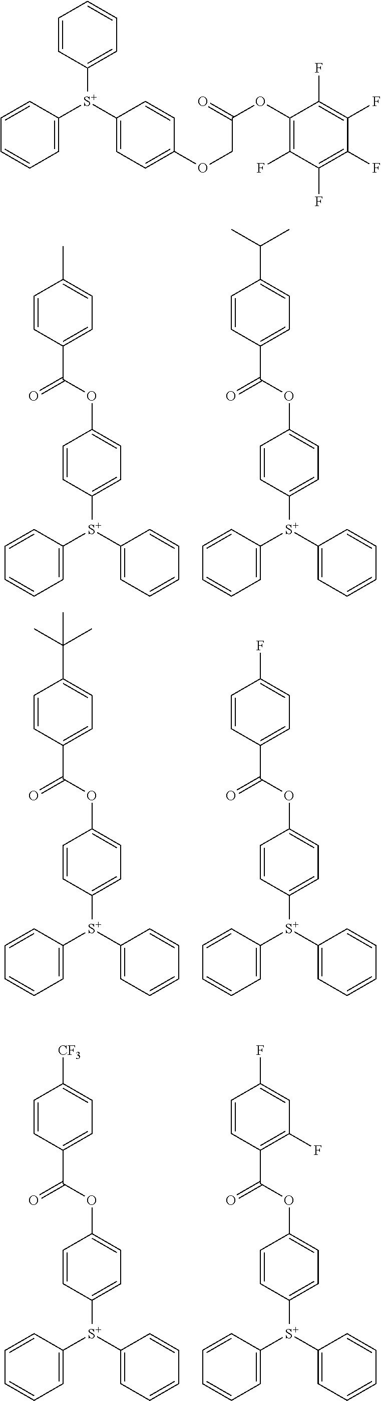

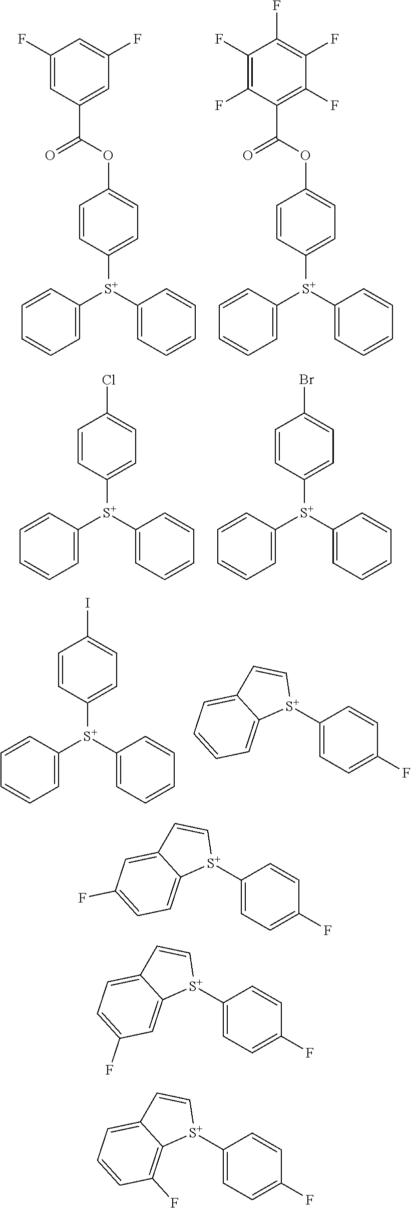

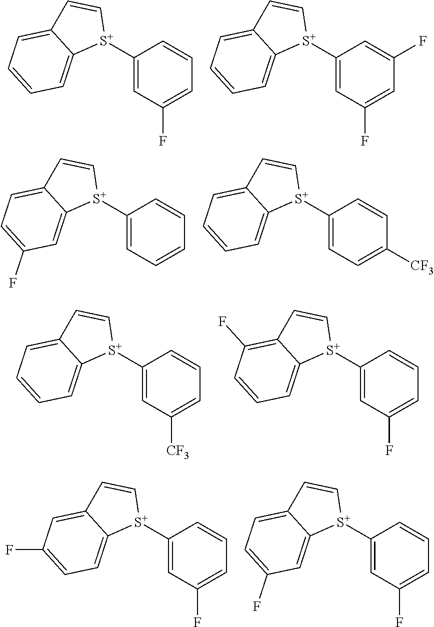

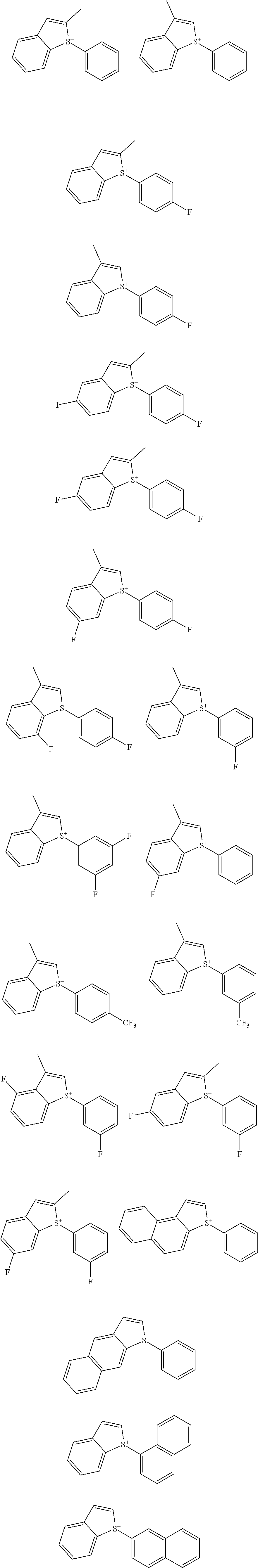

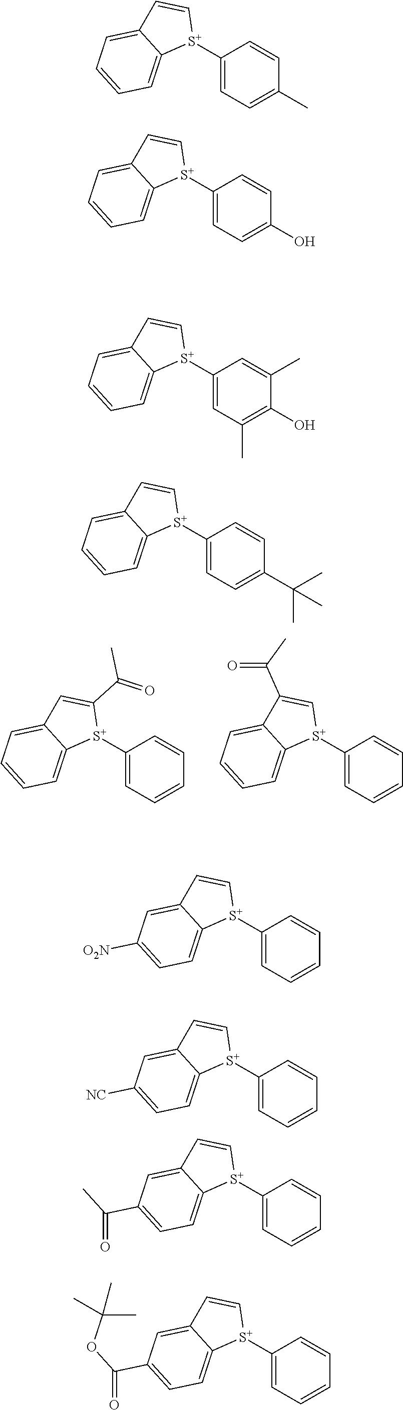

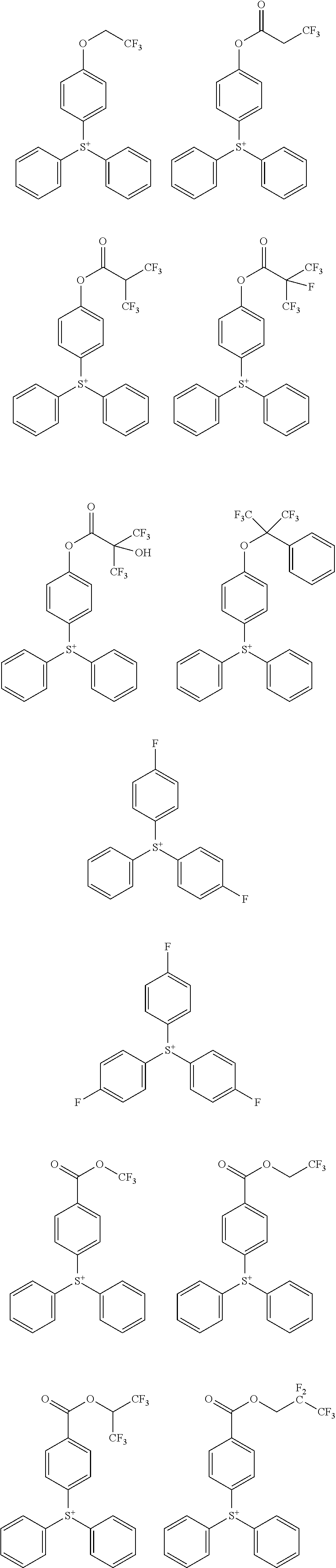

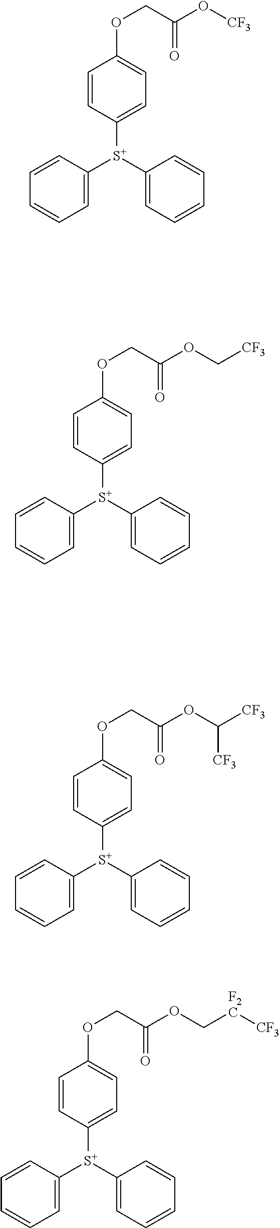

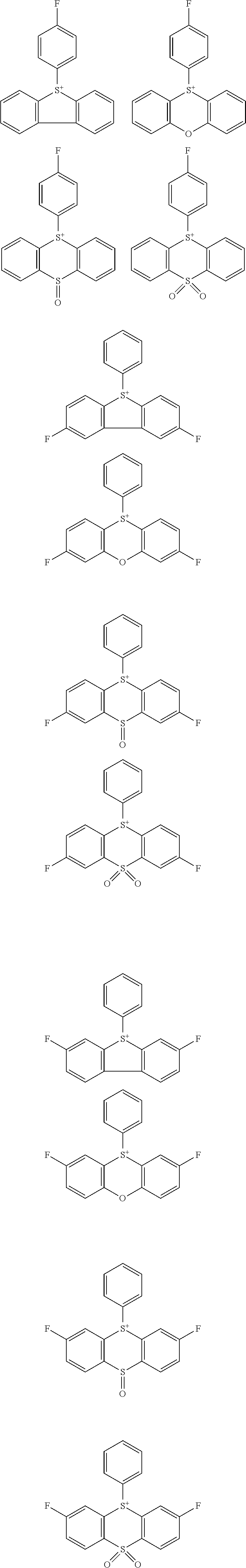

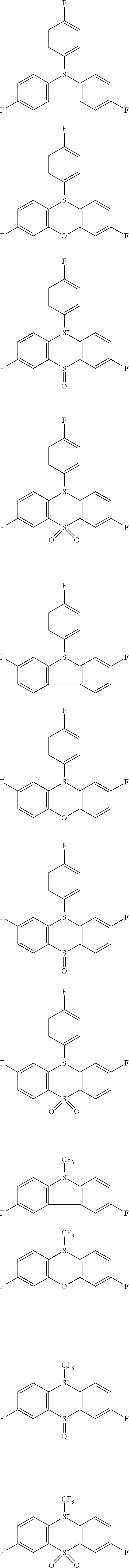

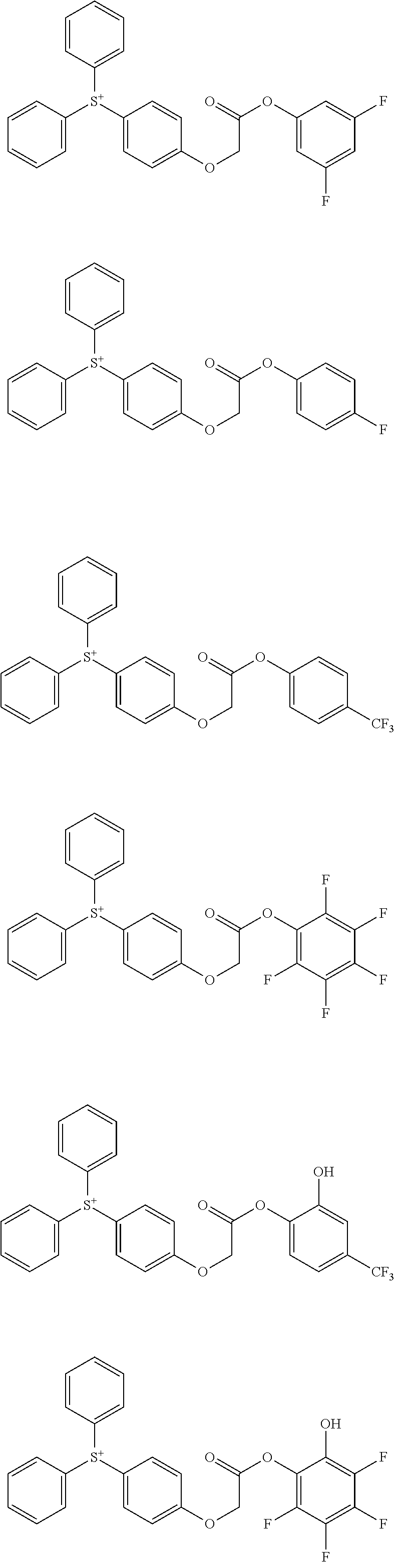

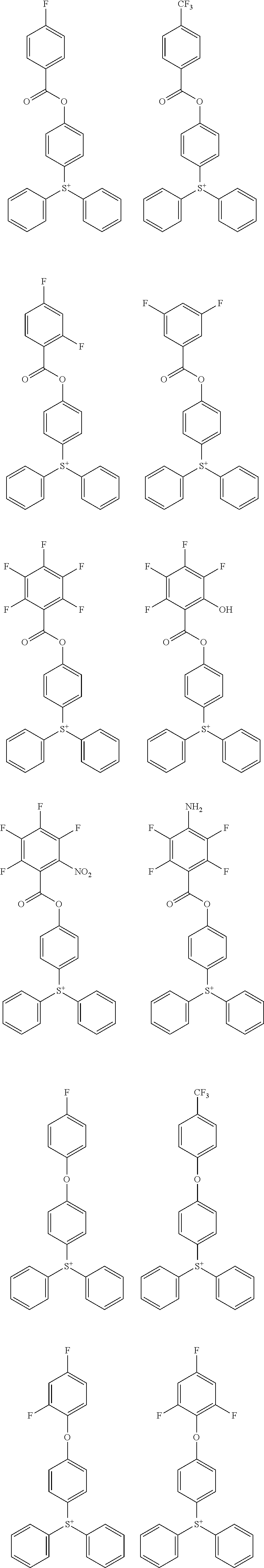

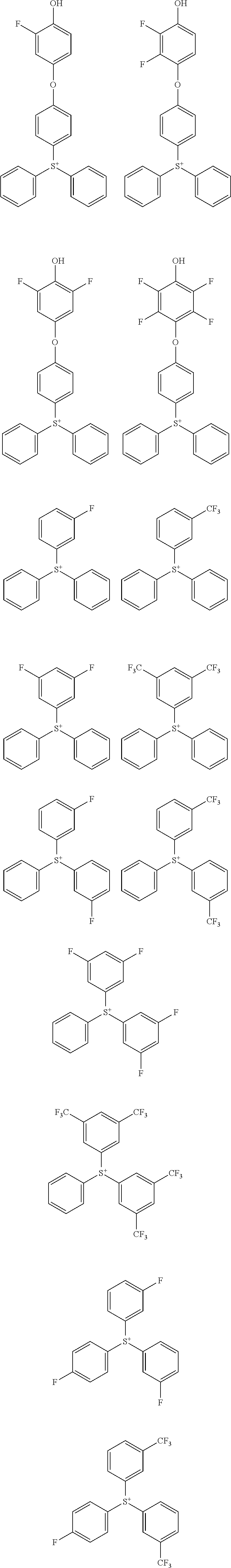

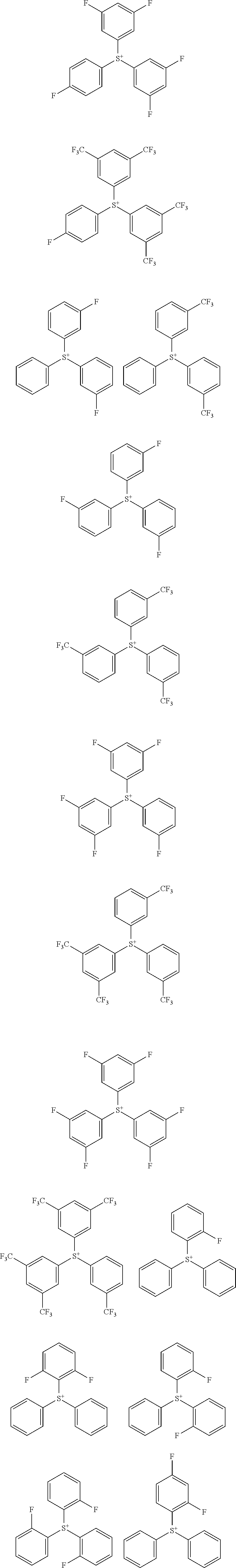

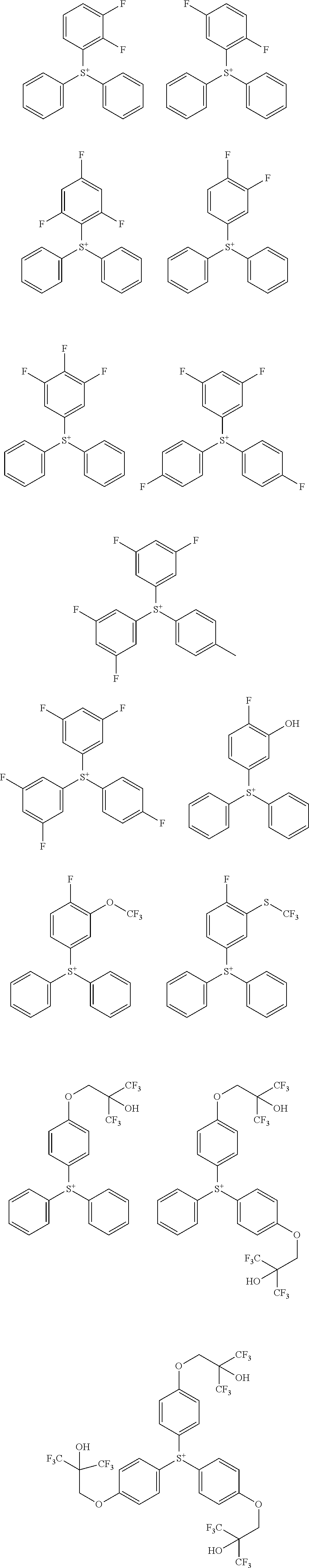

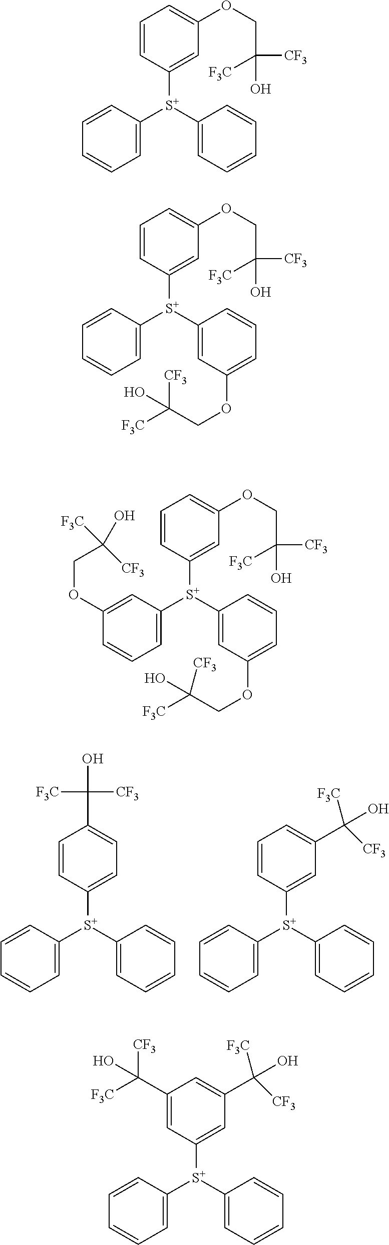

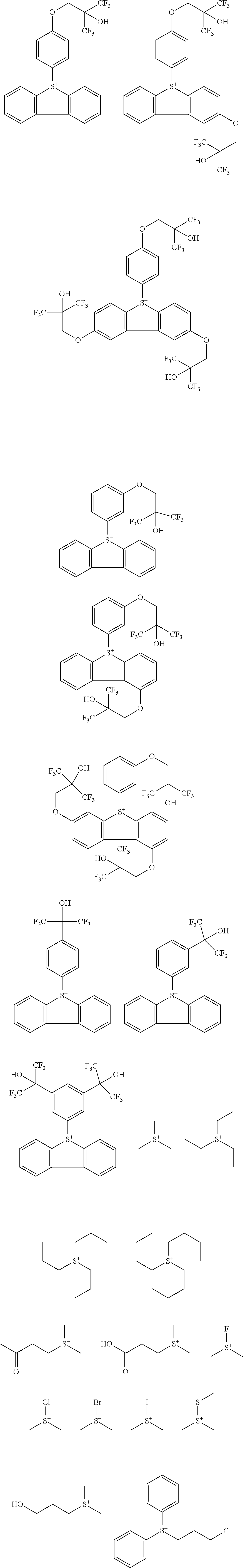

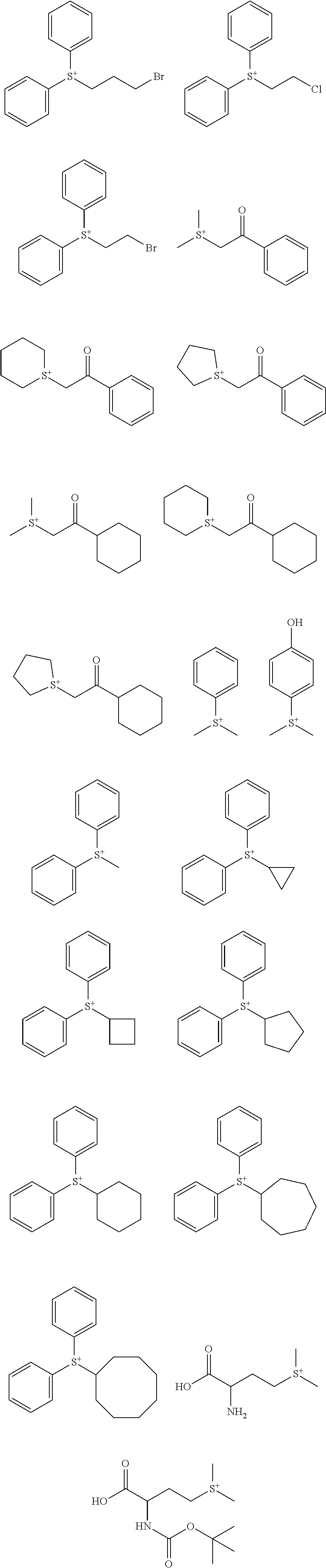

The sulfonium salt A can be synthesized by, for example, ion exchange of a hydrochloride salt or carbonate salt having the sulfonium cation with a substituted or unsubstituted hydroxytrifluoromethoxybenzoic acid or with a substituted or unsubstituted hydroxytrifluoromethylthiobenzoic acid.

In the inventive resist material, the content of the sulfonium salt A is preferably 0.001 to 50 parts by mass, and more preferably 0.01 to 40 parts by mass, relative to 100 parts by mass of the base polymer, described later. The sulfonium salt A may be used singly, or may be used in combination of two or more kinds thereof.

Base Polymer

The inventive resist material optionally comprises a base polymer. In a case of the positive-type resist material, the base polymer has a repeating unit having an acid-labile group. The repeating unit having an acid-labile group is preferably a repeating unit represented by the following general formula (a1) (hereinafter, also referred to as “repeating unit a1”) and/or a repeating unit represented by the following general formula (a2) (hereinafter, also referred to as “repeating unit a2”).

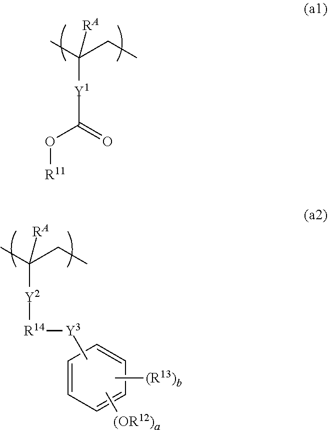

In the general formulae (a1) and (a2), R^{A} each independently represents a hydrogen atom or a methyl group. Y¹ represents a single bond, a phenylene group, a naphthylene group, or a linking group having 1 to 12 carbon atoms and having at least one selected from an ester bond and a lactone ring. Y² represents a single bond or an ester bond. Y³ represents a single bond, an ether bond, or an ester bond. R¹¹ and R¹² each independently represent an acid-labile group. When the base polymer has both of the repeating unit a1 and the repeating unit a2, R¹¹ and R¹² may be same as or different from each other. R¹³ represents a fluorine atom, a trifluoromethyl group, a cyano group, or a saturated hydrocarbyl group having 1 to 6 carbon atoms. R¹⁴ represents a single bond or an alkanediyl group having 1 to 6 carbon atoms, a part of carbon atoms therein being optionally substituted with an ether bond or an ester bond. “a” represents 1 or 2, “b” represents an integer of 0 to 4, and 1≤a+b≤5.

Examples of a monomer to yield the repeating unit a1 include the following monomers, but the monomer is not limited thereto. In the following formulae, R^{A} and R¹¹ represent the same as above.

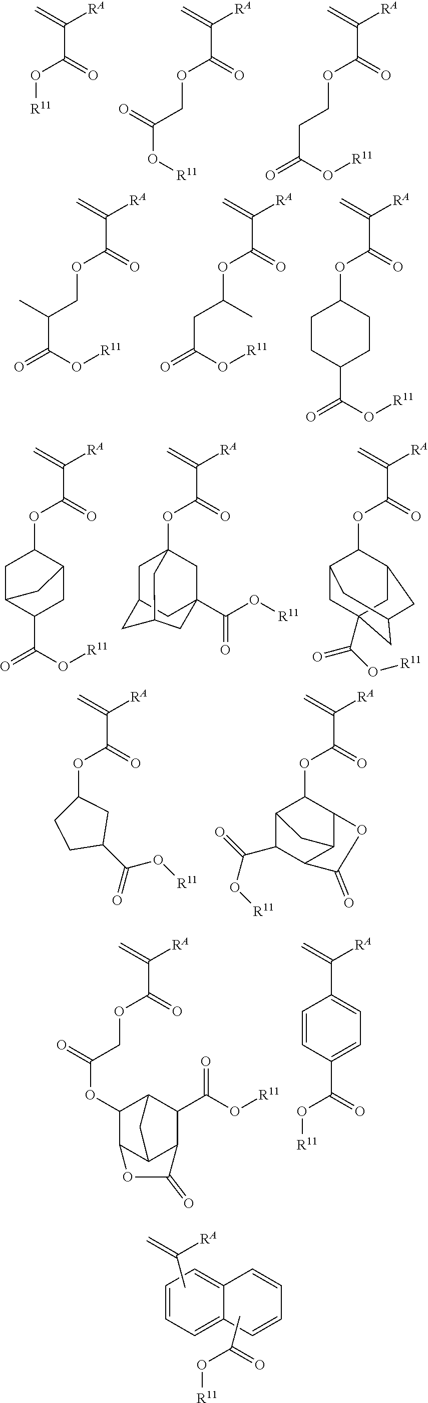

Examples of a monomer to yield the repeating unit a2 include the following monomers, but the monomer is not limited thereto. In the following formulae, R^{A} and R¹² represent the same as above.

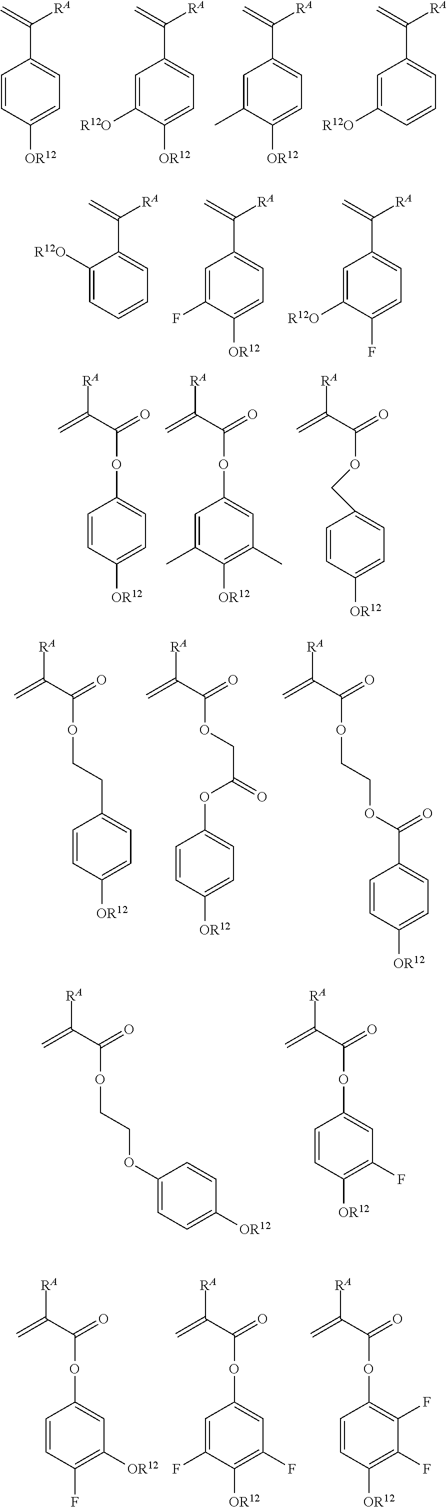

Examples of the acid-labile group represented by R¹, R^{1′}, R¹¹, and R¹² in the general formulae (1), (1′), (a1), and (a2) include groups described in JP 2013-80033 A and JP 2013-83821 A.

Typical examples of the acid-labile group include groups represented by the following general formulae (AL-1) to (AL-3).

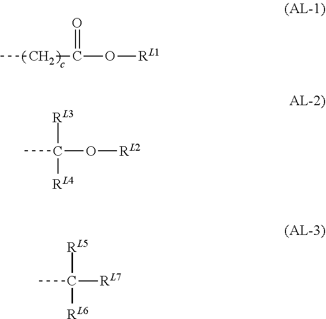

In the formulae, a broken line represents an attachment point.

In the general formulae (AL-1) and (AL-2), R^{L1} and R^{L2} each independently represent a hydrocarbyl group having 1 to 40 carbon atoms and optionally having a heteroatom such as an oxygen atom, a sulfur atom, a nitrogen atom, and a fluorine atom. The hydrocarbyl group may be a saturated or unsaturated group, and may be any of linear, branched, and cyclic groups. The hydrocarbyl group is preferably a saturated hydrocarbyl group having 1 to 40 carbon atoms, and more preferably a saturated hydrocarbyl group having 1 to 20 carbon atoms.

In the general formula (AL-1), “c” represents an integer of 0 to 10, and preferably an integer of 1 to 5.

In the general formula (AL-2), R^{L3} and R^{L4} each independently represent a hydrogen atom or a hydrocarbyl group having 1 to 20 carbon atoms and optionally having a heteroatom such as an oxygen atom, a sulfur atom, a nitrogen atom, and a fluorine atom. The hydrocarbyl group may be a saturated or unsaturated group, and may be any of linear, branched, and cyclic groups. The hydrocarbyl group is preferably a saturated hydrocarbyl group having 1 to 20 carbon atoms. Any two of R^{L2}, R^{L3}, and R^{L4} are optionally bonded to each other to form a ring having 3 to 20 carbon atoms together with the carbon atom or the carbon atom and oxygen atom to which these groups are bonded. The ring is preferably a ring having 4 to 16 carbon atoms, and particularly preferably an aliphatic ring.

In the formula (AL-3), R^{L5}, R^{L6}, and R^{L7} each independently represent a hydrocarbyl group having 1 to 20 carbon atoms and optionally having a heteroatom such as an oxygen atom, a sulfur atom, a nitrogen atom, and a fluorine atom. The hydrocarbyl group may be a saturated or unsaturated group, and may be any of linear, branched, and cyclic groups. The hydrocarbyl group is preferably a saturated hydrocarbyl group having 1 to 20 carbon atoms. Any two of R^{L5}, R^{L6}, and R^{L7} are optionally bonded to each other to form a ring having 3 to 20 carbon atoms together with the carbon atom to which these groups are bonded. The ring is preferably a ring having 4 to 16 carbon atoms, and particularly preferably an aliphatic ring.

When the base polymer in the resist material has the repeating unit a1 or a2, the resist material is a chemically amplified positive-type resist material.

The base polymer in the resist material also preferably has no acid-labile group. In this case, the resist material is a chemically amplified negative-type resist material.

The base polymer may have a repeating unit b having a phenolic hydroxy group as an adhesive group. Examples of a monomer to yield the repeating unit b include the following monomers, but the monomer is not limited thereto. In the following formulae, R A represents the same as above.

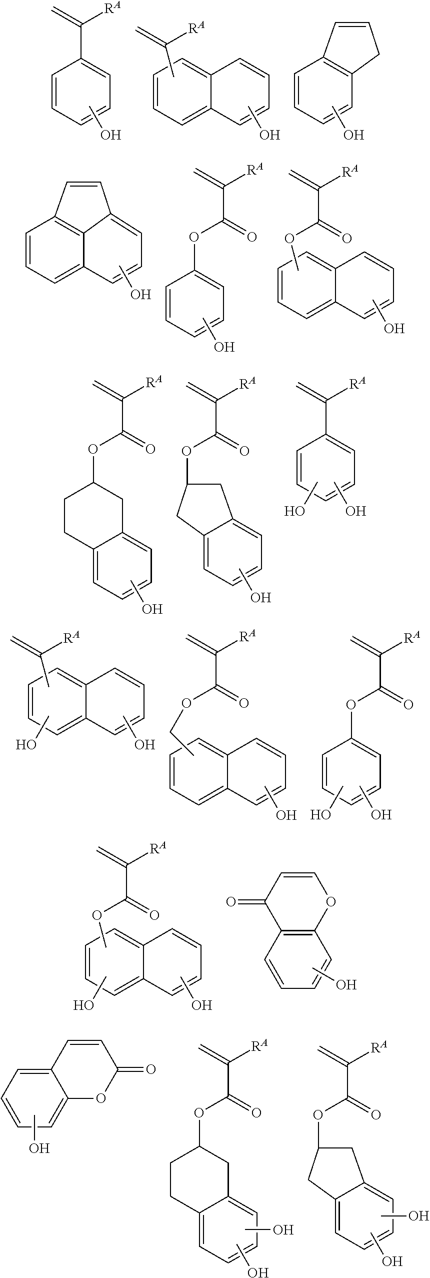

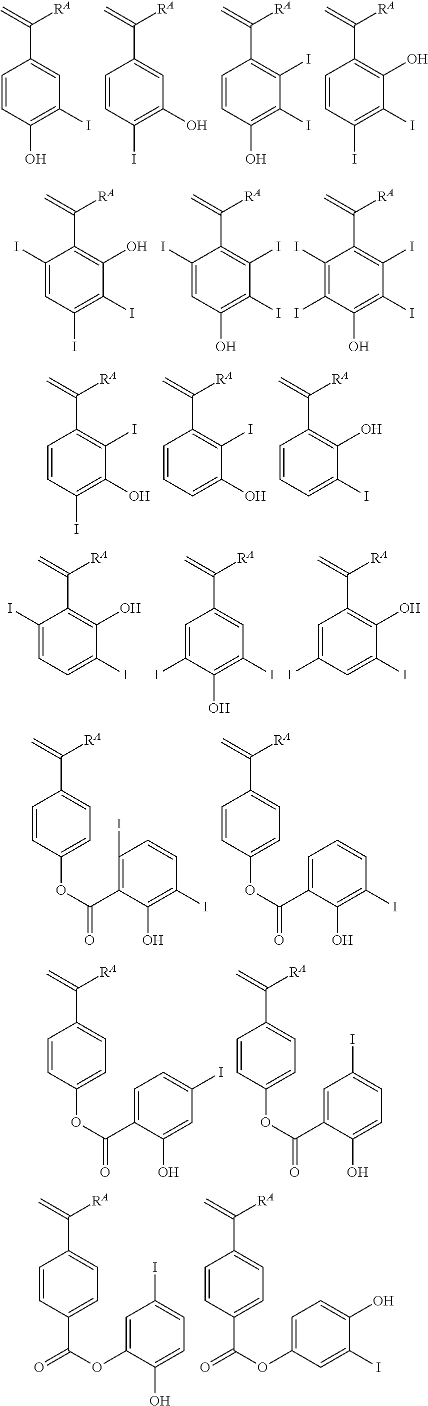

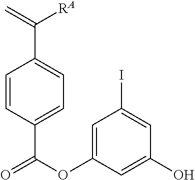

The base polymer may have a repeating unit c having, as another adhesive group, a hydroxy group other than a phenolic hydroxy group, a lactone ring, a sultone ring, an ether bond, an ester bond, a sulfonate ester bond, a carbonyl group, a sulfonyl group, a cyano group, and/or a carboxy group. Examples of a monomer to yield the repeating unit c include the following monomers, but the monomer is not limited thereto. In the following formulae, R A represents the same as above.

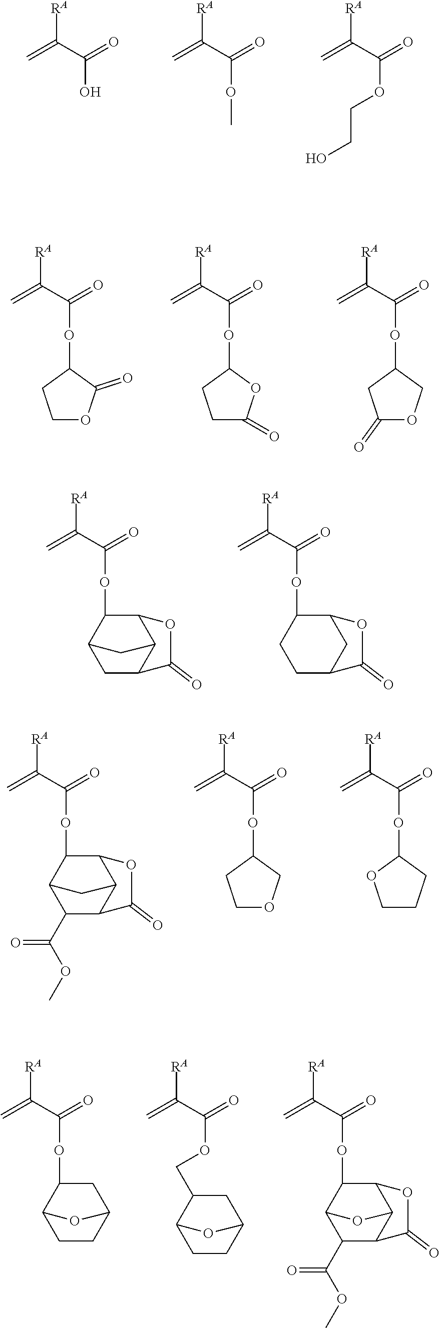

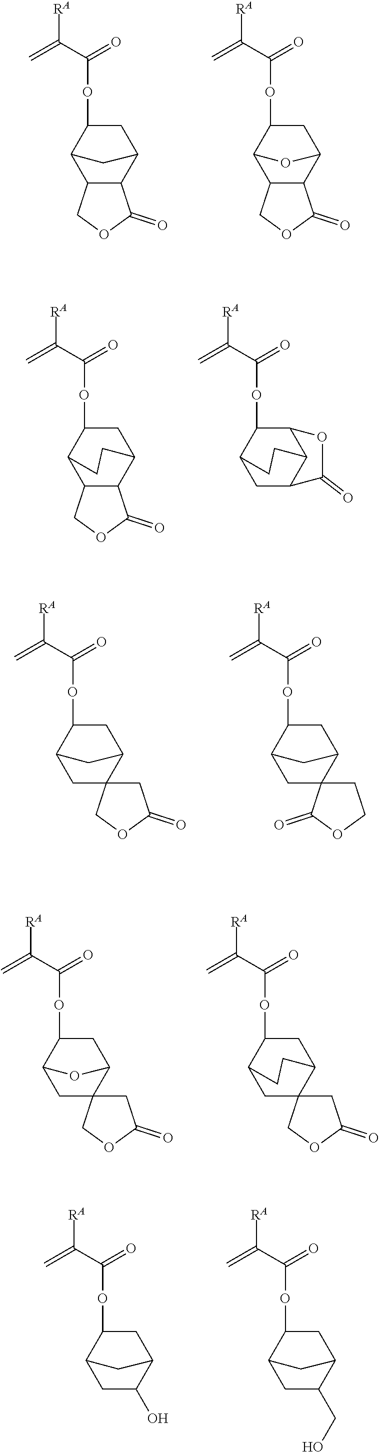

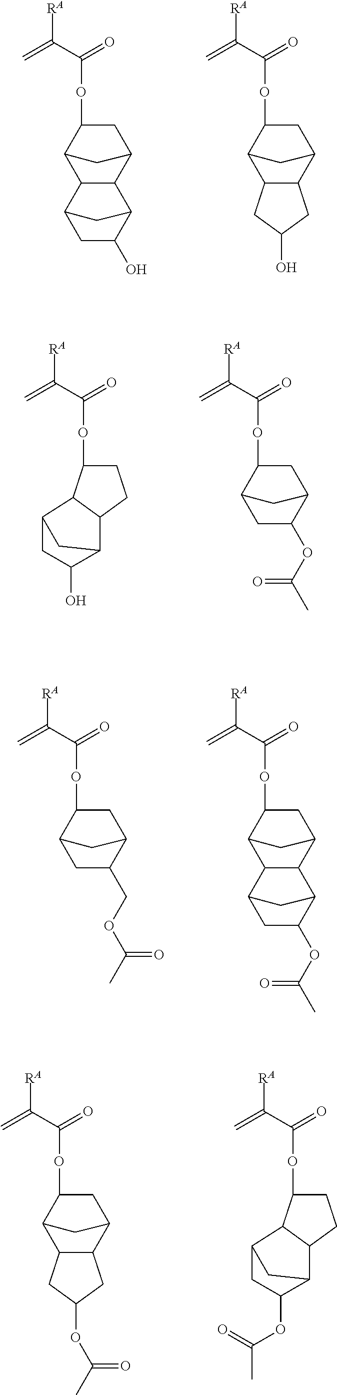

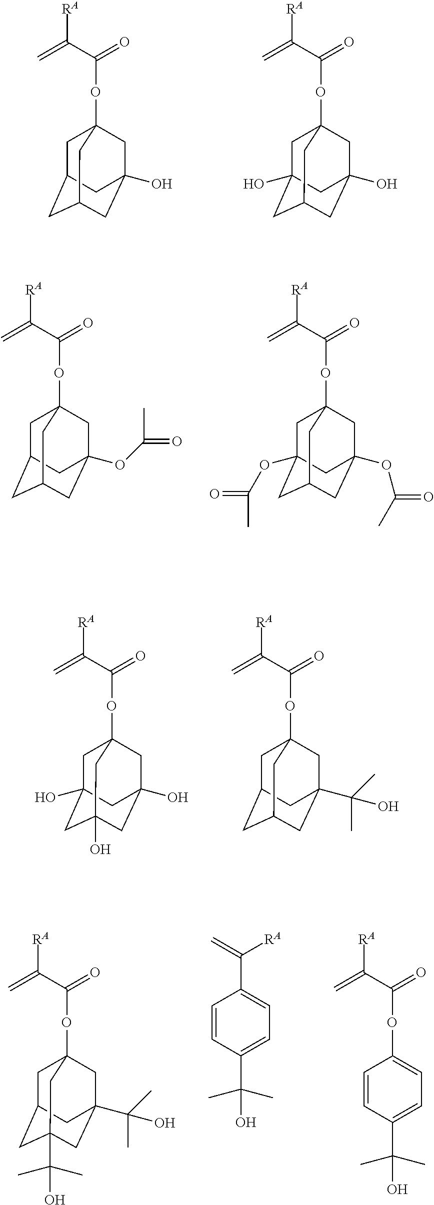

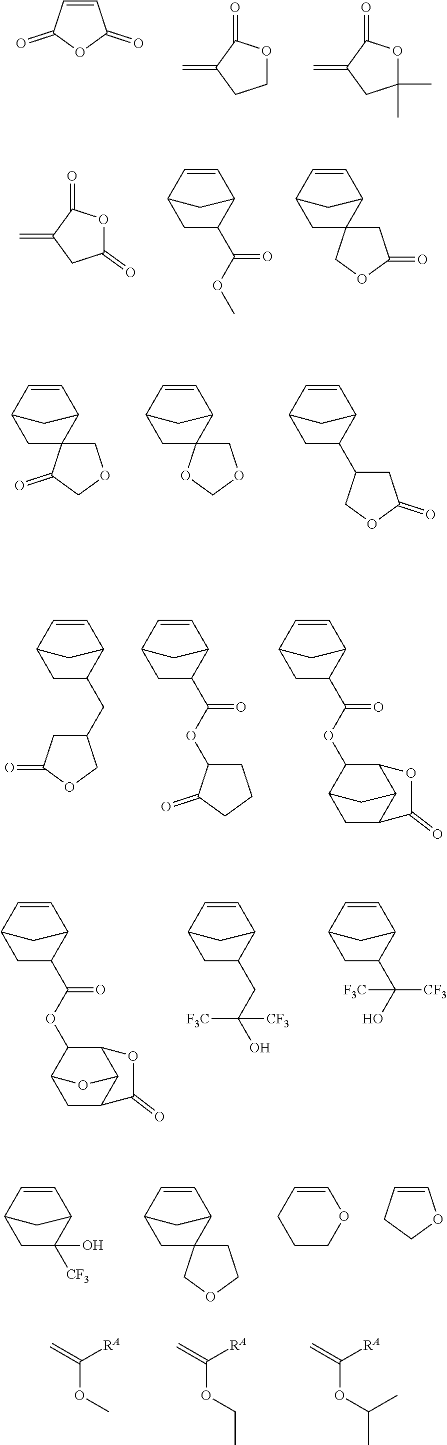

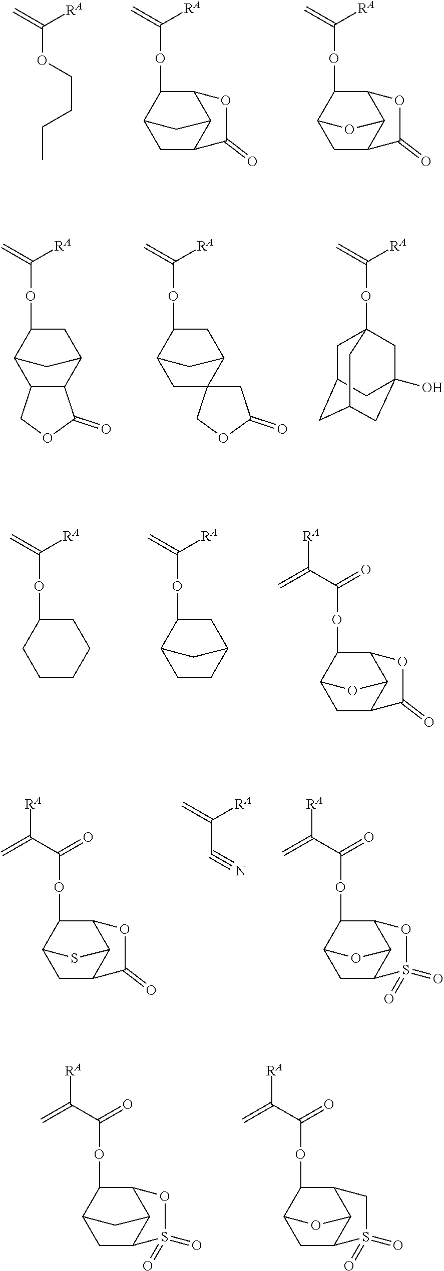

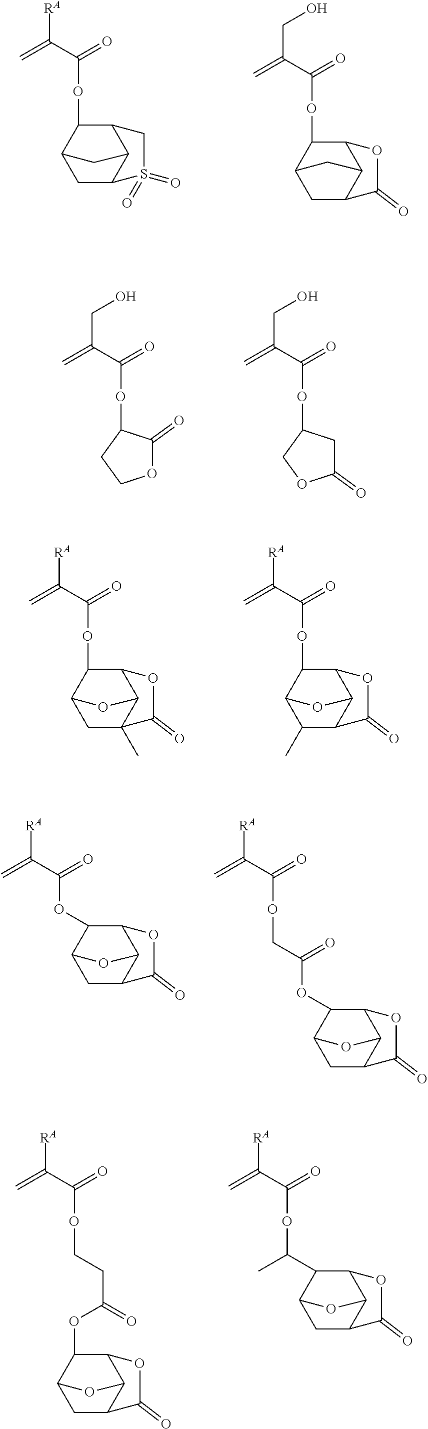

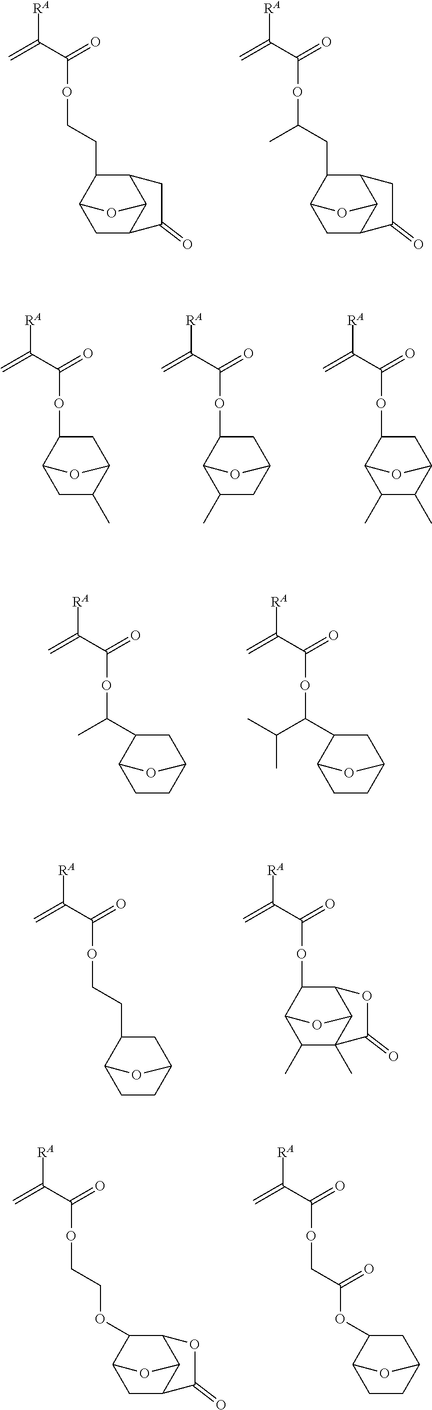

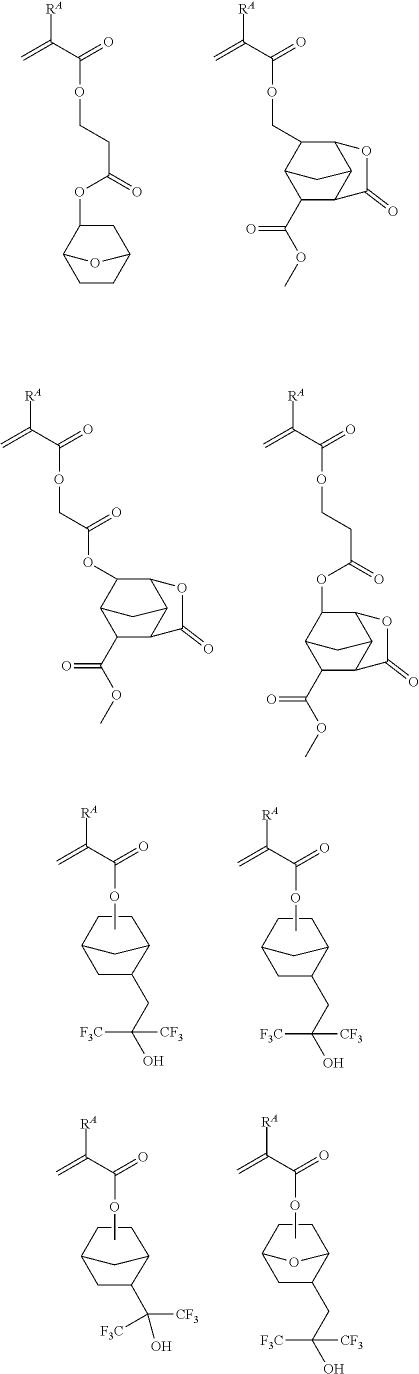

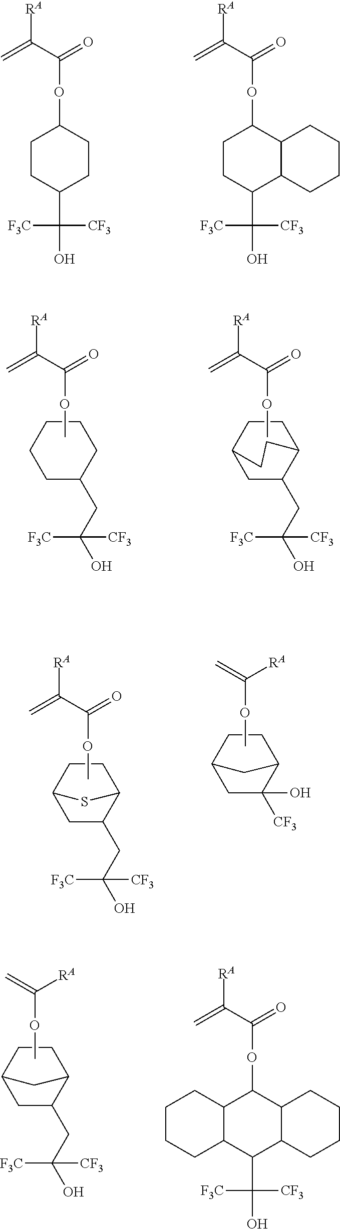

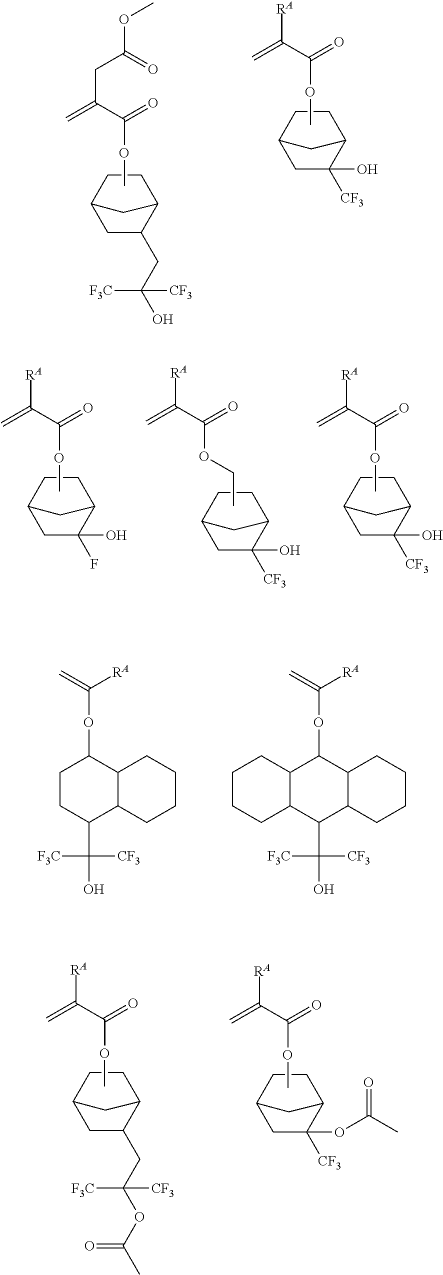

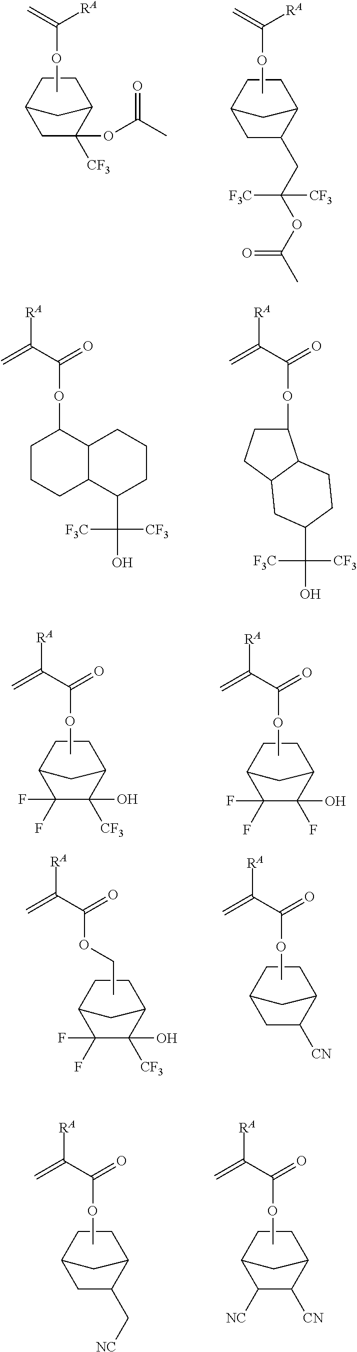

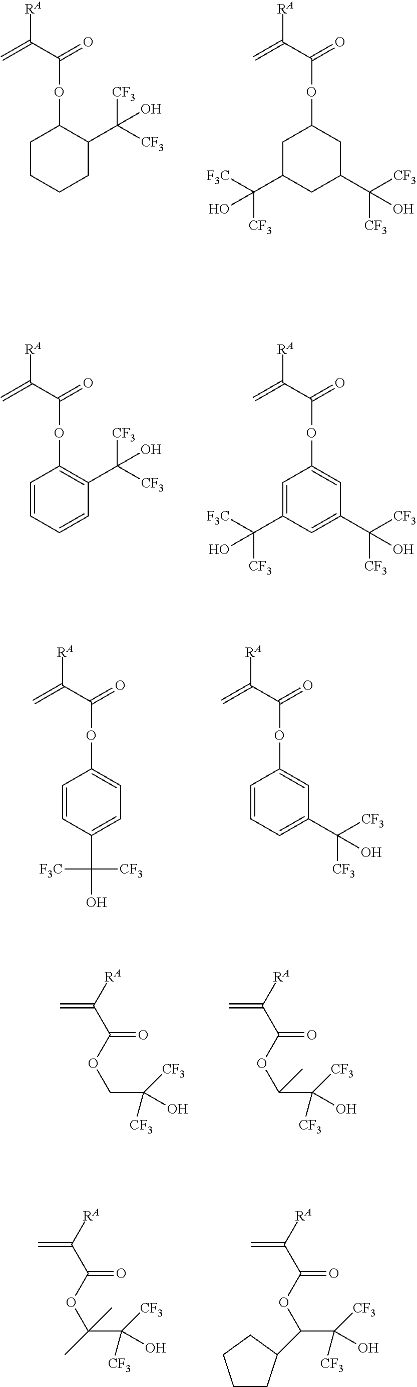

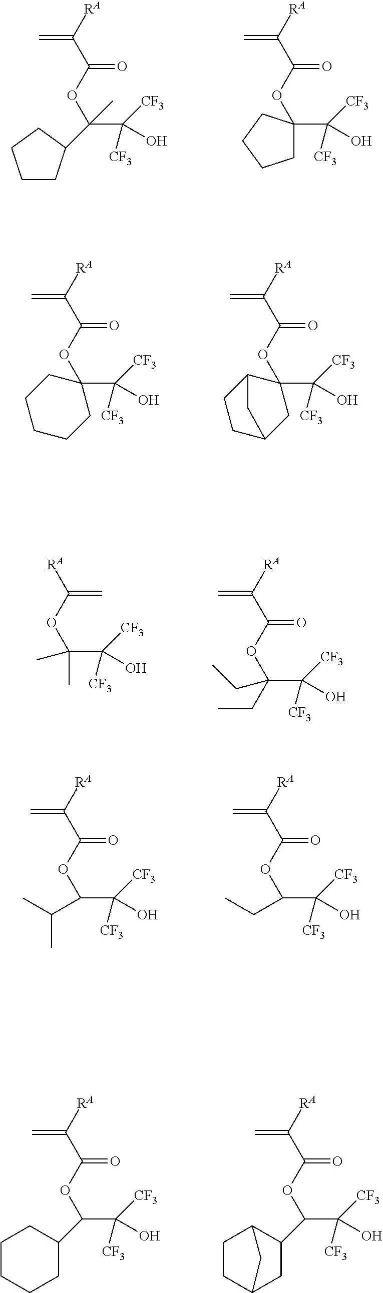

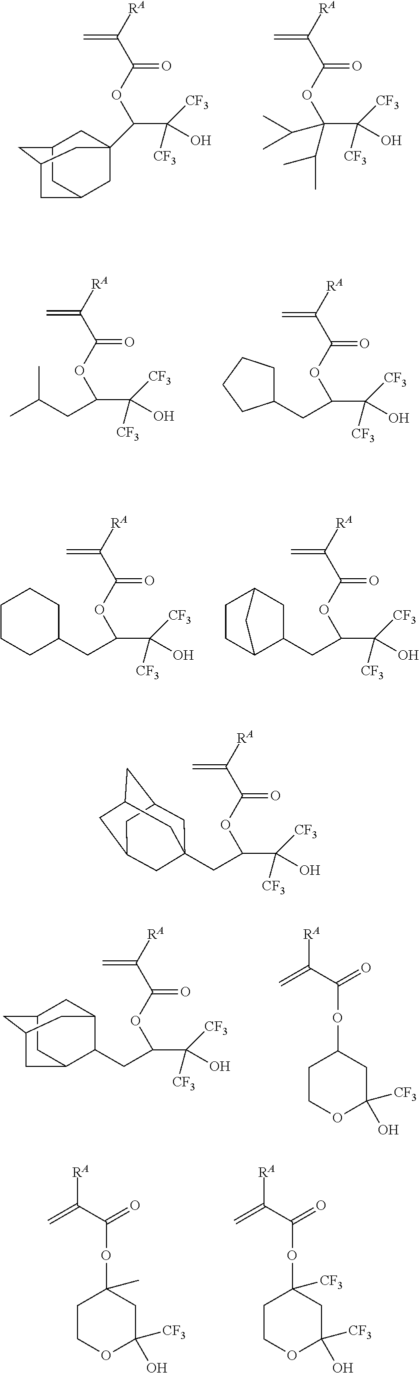

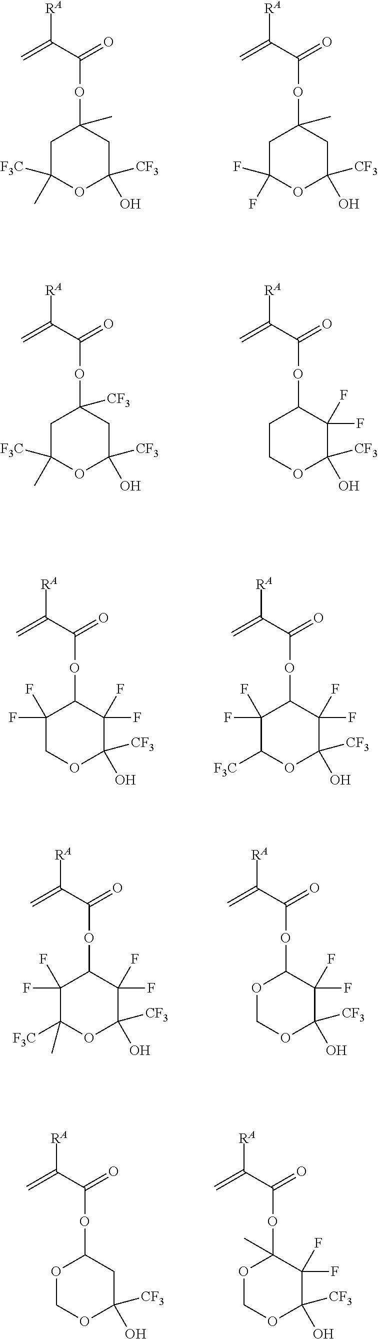

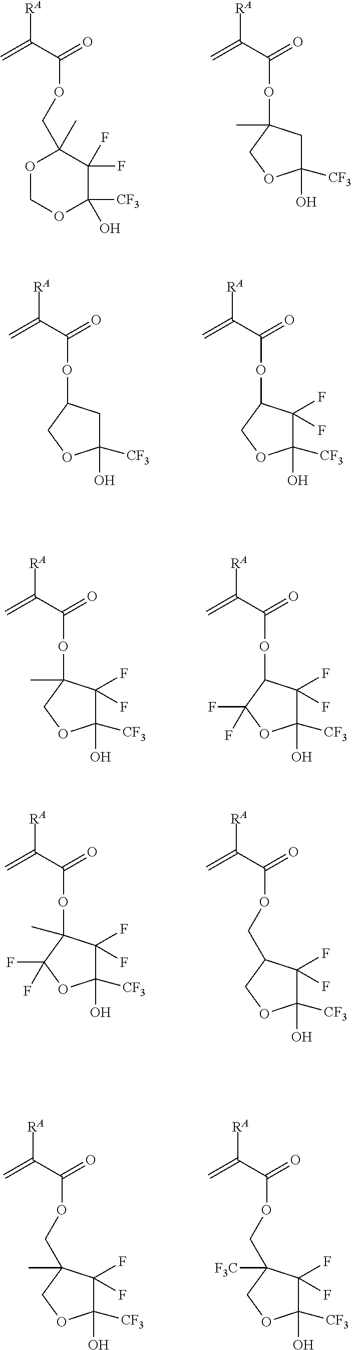

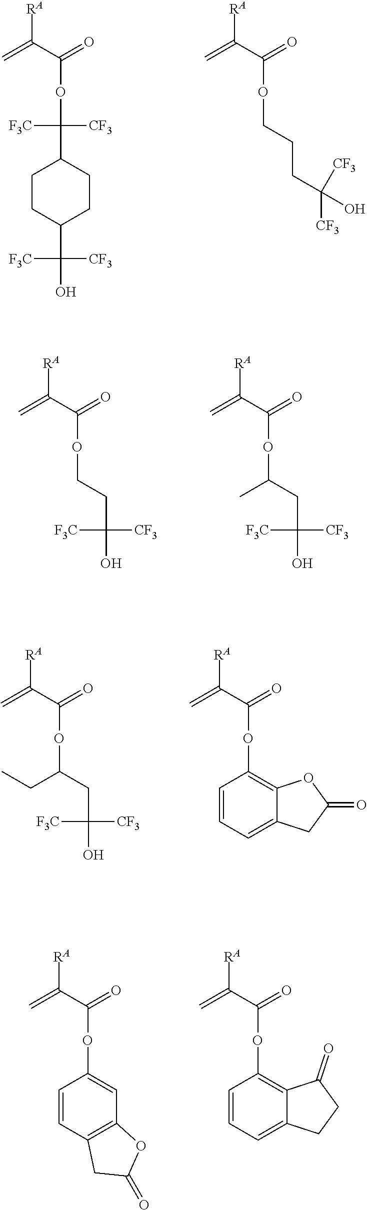

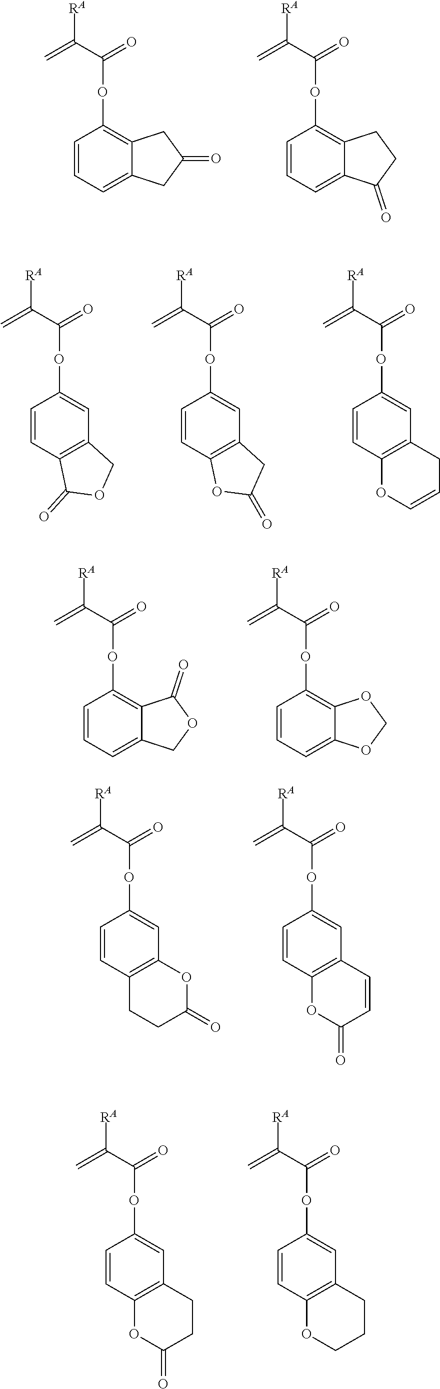

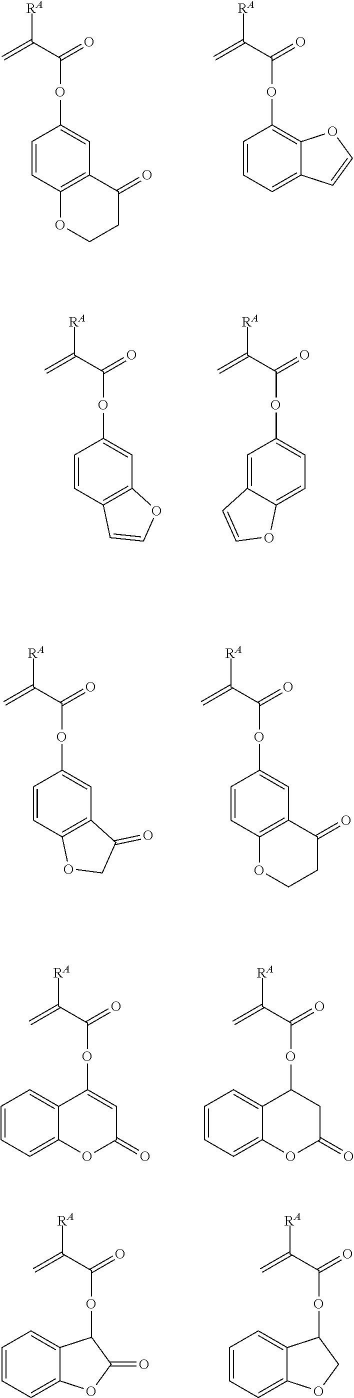

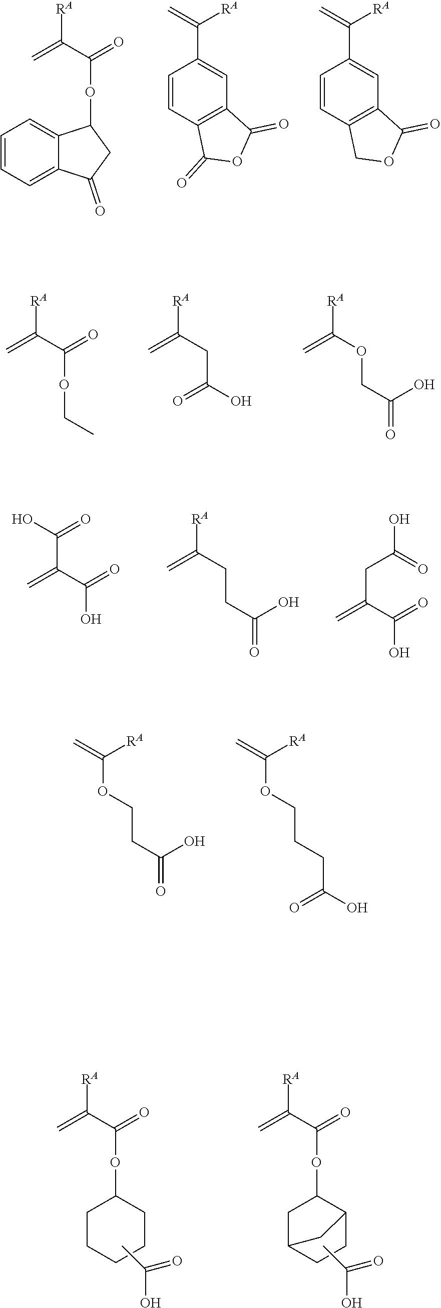

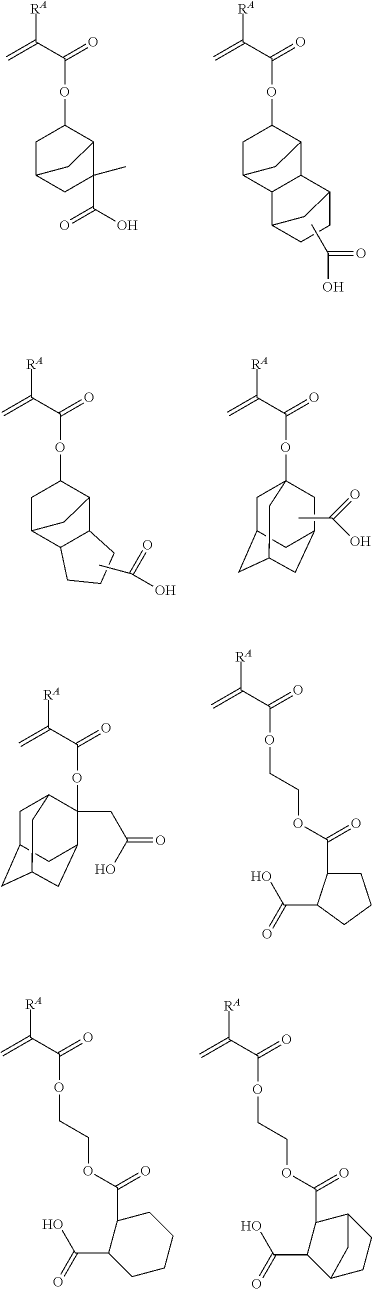

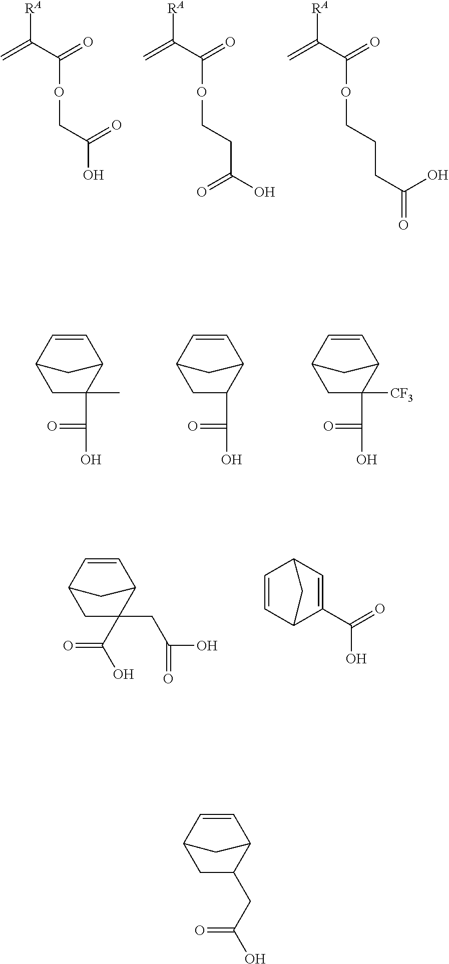

The base polymer may have a repeating unit d derived from indene, benzofuran, benzothiophene, acenaphthylene, chromone, coumarin, norbornadiene, or a derivative thereof. Examples of a monomer to yield the repeating unit d include the following monomers, but the monomer is not limited thereto.

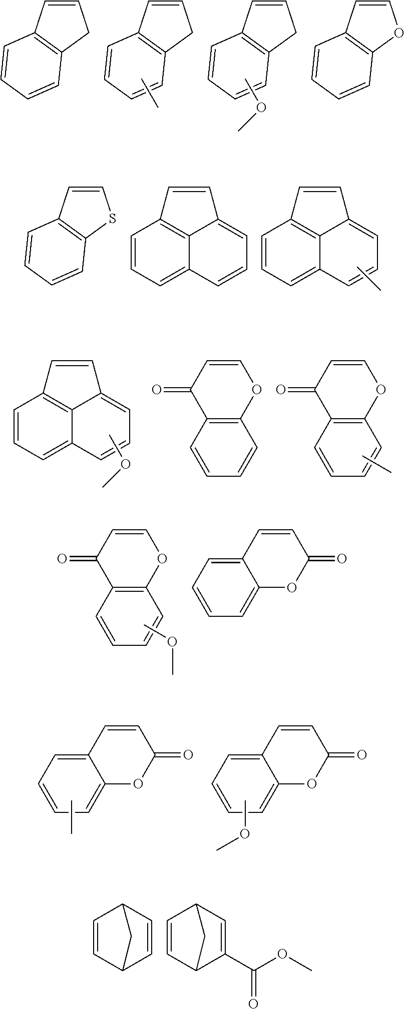

The base polymer may have a repeating unit e derived from styrene, vinylnaphthalene, vinylanthracene, vinylpyrene, methyleneindane, vinylpyridine, or vinylcarbazole.

The base polymer may have a repeating unit f derived from an onium salt having a polymerizable unsaturated bond. Examples of a preferable repeating unit f include a repeating unit represented by the following general formula (f1) (hereinafter, also referred to as the repeating unit f1), a repeating unit represented by the following general formula (f2) (hereinafter, also referred to as the repeating unit f2), and a repeating unit represented by the following general formula (f3) (hereinafter, also referred to as the repeating unit f3). The repeating units f1 to f3 may be used singly, or may be used in combination of two or more kinds thereof.

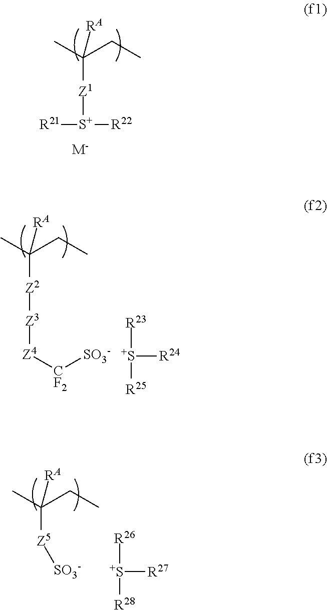

In the general formulae (f1) to (f3), R^{A} each independently represents a hydrogen atom or a methyl group. Z¹ represents a single bond, an aliphatic hydrocarbylene group having 1 to 6 carbon atoms, a phenylene group, a naphthylene group, an ester bond, a group having 7 to 18 carbon atoms obtained by combining these groups, —O—Z¹¹—, —C(═O)—O—Z¹¹—, or —C(═O)—NH—Z¹¹—. Z¹¹ represents an aliphatic hydrocarbylene group having 1 to 6 carbon atoms, a phenylene group, a naphthylene group, or a group having 7 to 18 carbon atoms obtained by combining these groups, Z″ optionally having a carbonyl group, an ester bond, an ether bond, or a hydroxy group. Z² represents a single bond or an ester bond. Z³ represents a single bond, —Z³¹—C(═O)—O—, —Z³¹—O—, or —Z³¹—O—C(═O)—. Z³¹ represents a hydrocarbylene group having 1 to 12 carbon atoms, a phenylene group, or a group having 7 to 18 carbon atoms obtained by combining these groups, Z³¹ optionally having a carbonyl group, an ester bond, an ether bond, an iodine atom, or a bromine atom. Z⁴ represents a methylene group, a 2,2,2-trifluoro-1,1-ethanediyl group, or a carbonyl group. Z⁵ represents a single bond, a methylene group, an ethylene group, a phenylene group, a fluorinated phenylene group, a phenylene group substituted with a trifluoromethyl group, —O—Z⁵¹—, —C(═O)—O—Z⁵¹—, or —C(═O)—NH—Z⁵¹—. Z⁵¹ represents an aliphatic hydrocarbylene group having 1 to 15, preferably 1 to 6, carbon atoms, a phenylene group, a fluorinated phenylene group, a phenylene group substituted with a trifluoromethyl group, or a combination thereof, Z⁵¹ optionally having a carbonyl group, an ester bond, an ether bond, a halogen atom, and/or a hydroxy group.

In the general formulae (f1) to (f3), R²¹ to R²⁸ each independently represent a halogen atom or a hydrocarbyl group having 1 to 25, preferably 1 to 20, carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R² to R⁴ in the description of the general formulae (1) and (1′). A part or all of hydrogen atoms in the hydrocarbyl group are optionally substituted with a group having a heteroatom such as an oxygen atom, a sulfur atom, a nitrogen atom, and a halogen atom. A part of carbon atoms in these groups is optionally substituted with a group having a heteroatom such as an oxygen atom, a sulfur atom, and a nitrogen atom. As a result, optionally contained are a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride, a haloalkyl group, etc. R²³ and R²⁴ or R²⁶ and R²⁷ are optionally bonded to each other to form a ring together with the sulfur atom to which these groups are bonded. In this case, examples of the ring include rings same as those exemplified as the rings that can be formed by bonding R² and R³ together with the sulfur atom to which these groups are bonded, described in the general formulae (1) and (1′).

In the general formula (f1), M⁻ represents a non-nucleophilic counterion. Examples of the non-nucleophilic counterion include: halide ions, such as a chloride ion and a bromide ion; fluoroalkylsulfonate ions, such as a triflate ion, a 1,1,1-trifluoroethanesulfonate ion, and a nonafluorobutanesulfonate ion; arylsulfonate ions, such as a tosylate ion, a benzenesulfonate ion, a 4-fluorobenzenesulfonate ion, and a 1,2,3,4,5-pentafluorobenzenesulfonate ion; alkylsulfonate ions, such as a mesylate ion and a butanesulfonate ion; imide ions, such as a bis(trifluoromethylsulfonyl)imide ion, a bis(perfluoroethylsulfonyl)imide ion, and a bis(perfluorobutylsulfonyl)imide ion; and methide ions, such as a tris(trifluoromethylsulfonyl)methide ion and a tris(perfluoroethylsulfonyl)methide ion.

Other examples of the non-nucleophilic counterion include: a sulfonate ion in which the α-position is substituted with a fluorine atom, represented by the following general formula (f1-1); and a sulfonate ion in which the α-position is substituted with a fluorine atom and the 0-position is substituted with a trifluoromethyl group, represented by the following general formula (f1-2).

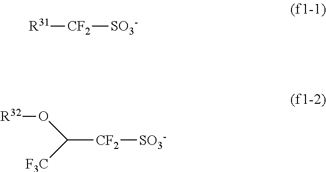

In the general formula (f1-1), R³¹ represents a hydrogen atom or a hydrocarbyl group having 1 to 20 carbon atoms. The hydrocarbyl group optionally has an ether bond, an ester bond, a carbonyl group, a lactone ring, or a fluorine atom. The hydrocarbyl group may be a saturated or unsaturated group, and may be any of linear, branched, and cyclic groups. Specific examples thereof include groups same as those exemplified as a hydrocarbyl group represented by R¹¹¹ in the formula (3A′), described later.

In the general formula (f1-2), R³² represents a hydrogen atom, a hydrocarbyl group having 1 to 30 carbon atoms, or a hydrocarbylcarbonyl group having 6 to 20 carbon atoms. The hydrocarbyl group and the hydrocarbylcarbonyl group optionally have an ether bond, an ester bond, a carbonyl group, or a lactone ring. The hydrocarbyl group and the hydrocarbyl part of the hydrocarbylcarbonyl group may be a saturated or unsaturated group, and may be any of linear, branched, and cyclic groups. Specific examples thereof include groups same as those exemplified as a hydrocarbyl group represented by R¹¹¹ in the formula (3A′), described later.

Examples of the cation of a monomer to yield the repeating unit f1 include the following cations, but the cation is not limited thereto. In the following formulae, R A represents the same as above.

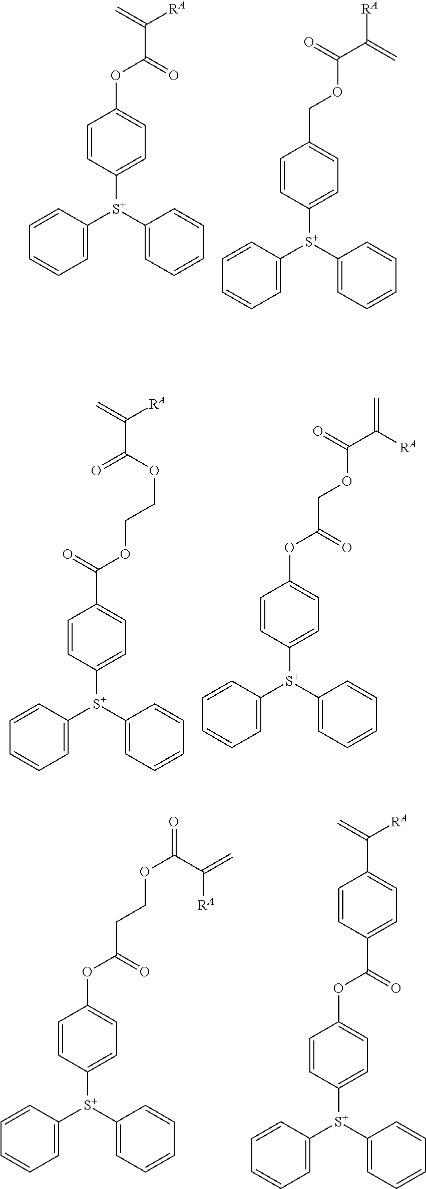

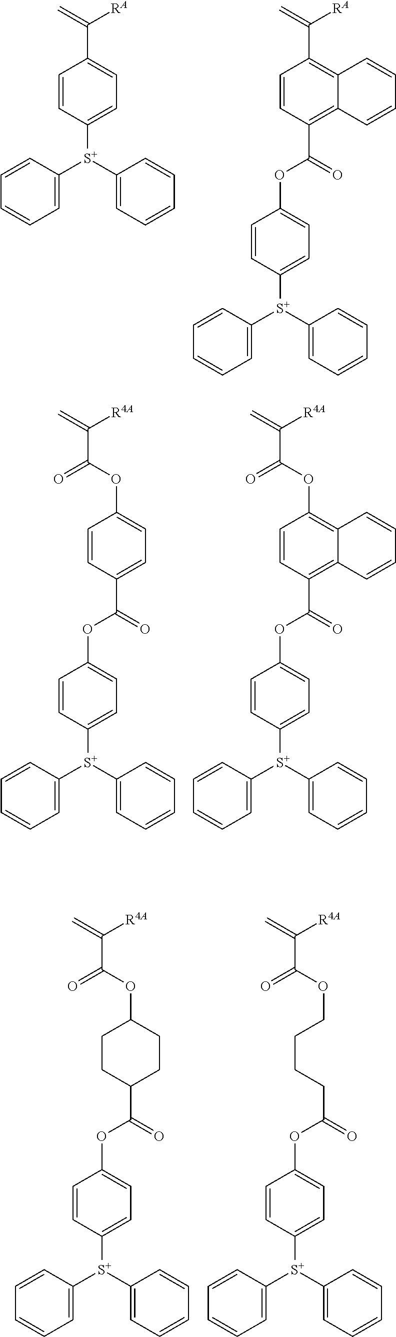

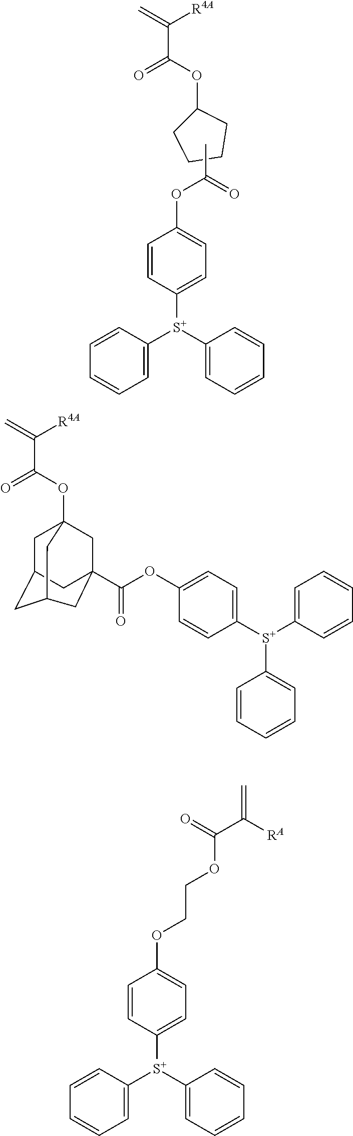

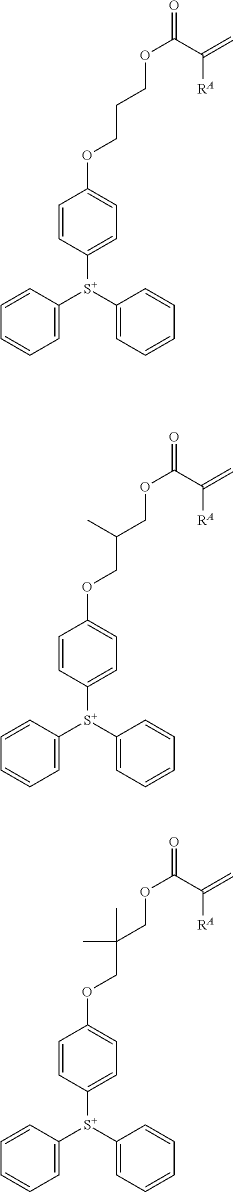

Specific examples of the cation of a monomer to yield the repeating unit f2 or f3 include cations same as those exemplified as the cation of the sulfonium salt represented by the formulae (1) and (1′).

Examples of the anion of a monomer to yield the repeating unit f2 include the following anions, but the anion is not limited thereto. In the following formulae, R A represents the same as above.

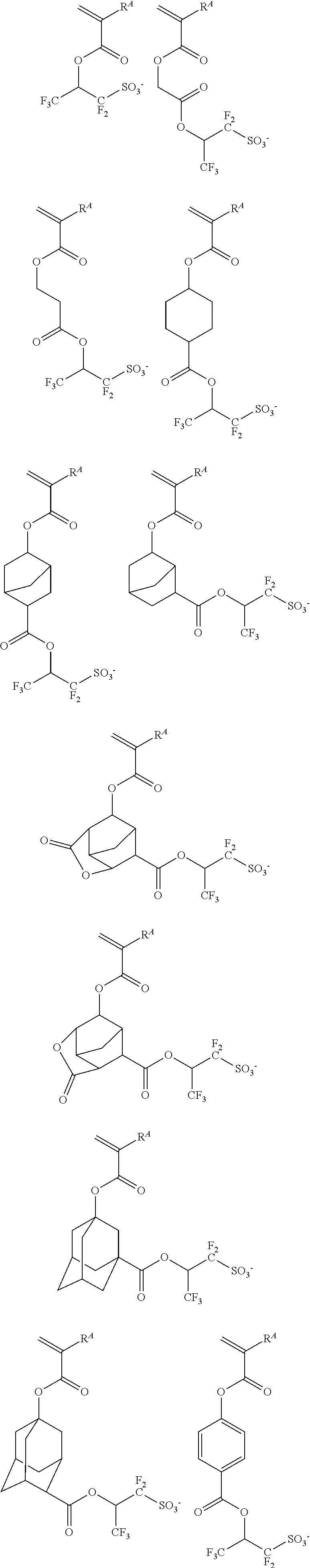

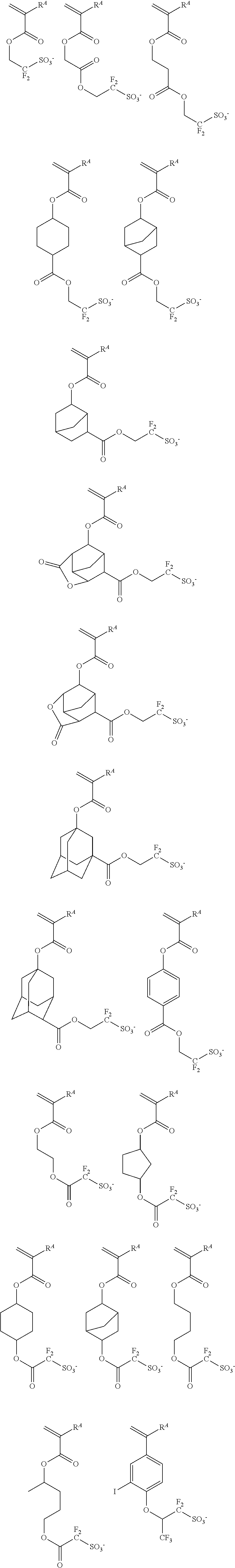

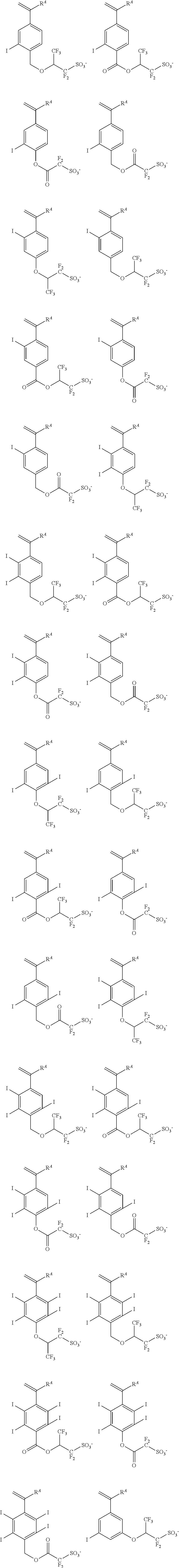

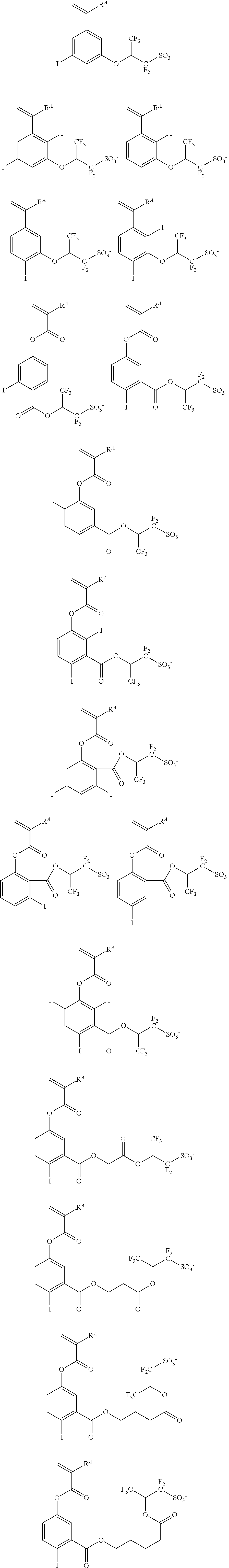

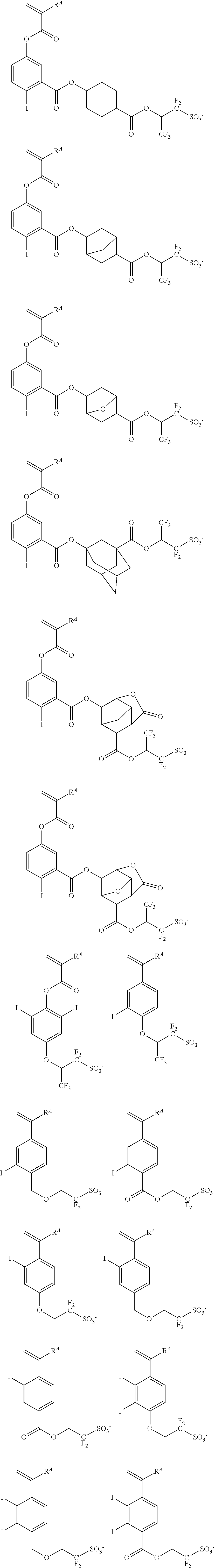

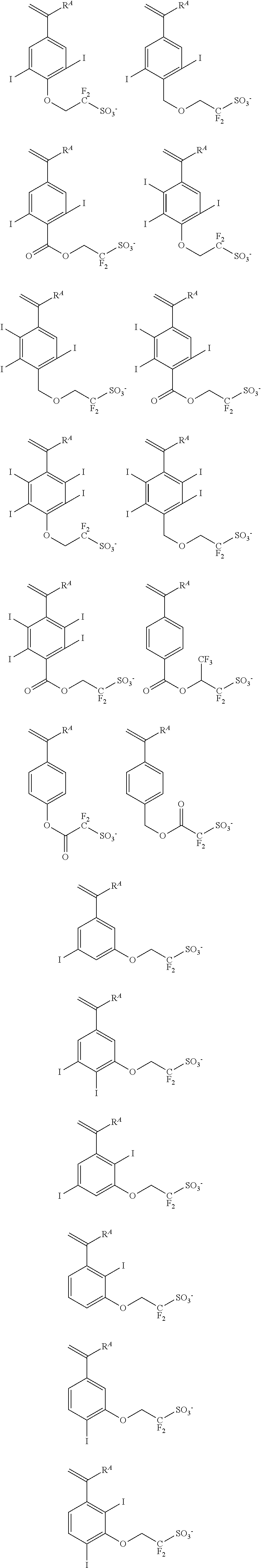

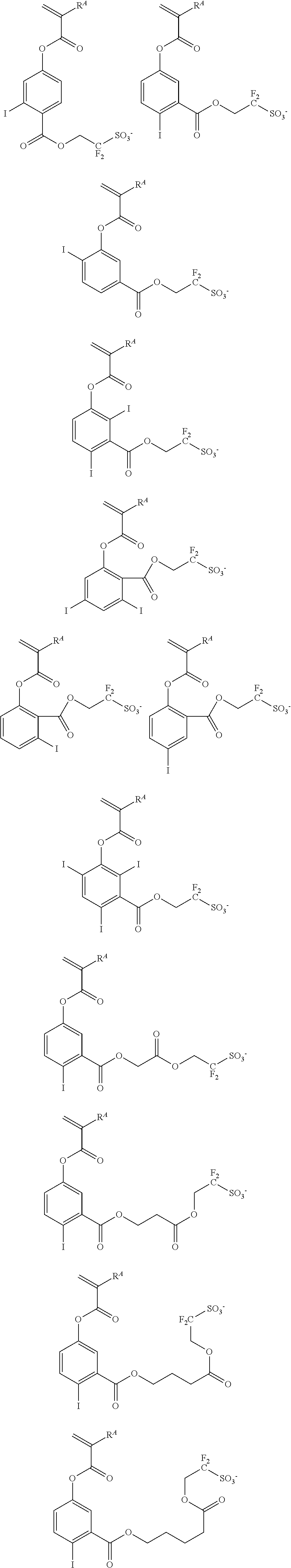

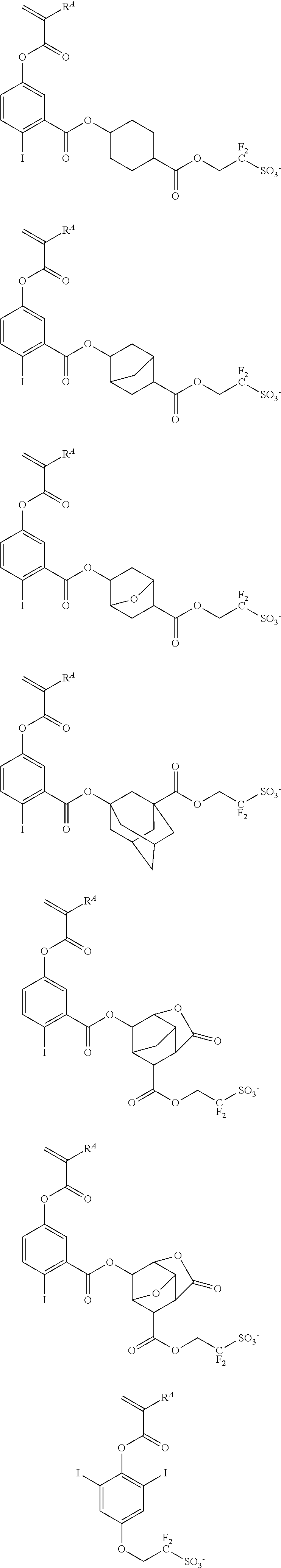

Examples of the anion of a monomer to yield the repeating unit f3 include the following anions, but the anion is not limited thereto. In the following formulae, R^{A} represents the same as above.

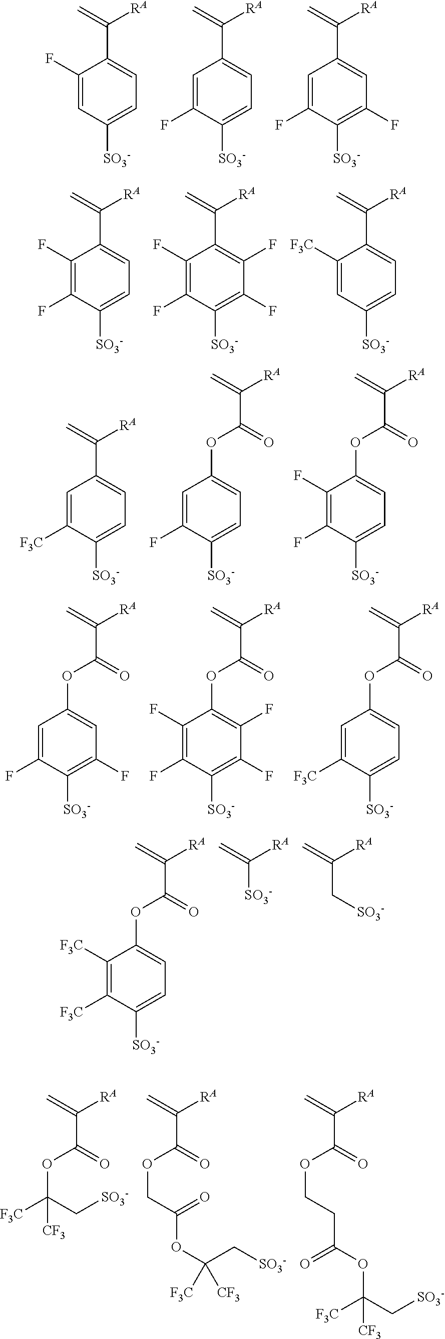

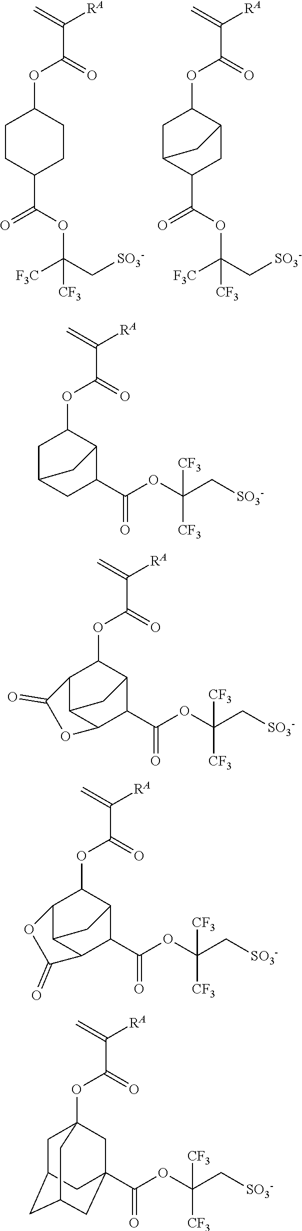

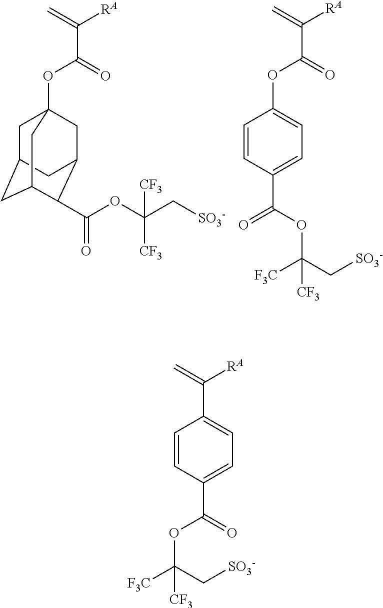

The repeating units f1 to f3 have a function of an acid generator. Binding the acid generator to the polymer main chain reduces the acid diffusion, and can prevent deterioration of resolution due to blur with the acid diffusion. In addition, uniformly dispersing the acid generator improves LWR and CDU. When the base polymer having the repeating unit f is used, blending an additive-type acid generator, described later, can be omitted.

In the base polymer, content rates of the repeating units a1, a2, b, c, d, e, f1, f2, and f3 are preferably 0≤a1≤0.9, 0≤a2≤0.9, 0≤a1+a2≤0.9, 0≤b≤0.9, 0≤c≤0.9, 0≤d≤0.5, 0≤e≤0.5, 0≤f1≤0.5, 0≤f2≤0.5, 0≤f3≤0.5, and 0≤f1+f2+f3≤0.5, more preferably 0≤a1≤0.8, 0≤a2≤0.8, 0≤a1+a2≤0.8, 0≤b≤0.8, 0≤c≤0.8, 0≤d≤0.4, 0≤e≤0.4, 0≤f1≤0.4, 0≤f2≤0.4, 0≤f3≤0.4, and 0≤f1+f2+f3≤0.4, and further preferably 0≤a1≤0.7, 0≤a2≤0.7, 0≤a1+a2≤0.7, 0≤b≤0.7, 0≤c≤0.7, 0≤d≤0.3, 0≤e≤0.3, 0≤f1≤0.3, 0≤f2≤0.3, 0≤f3≤0.3, and 0≤f1+f2+f3≤0.3. Note that, a1+a2+b+c+d+f1+f2+f3+e=1.0.

To synthesize the base polymer, the monomers to yield the aforementioned repeating units are heated in an organic solvent with adding a radical polymerization initiator to perform polymerization, for example.

Examples of the organic solvent used for the polymerization include toluene, benzene, tetrahydrofuran (THF), diethyl ether, and dioxane. Examples of the polymerization initiator include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. The temperature for the polymerization is preferably 50 to 80° C. The reaction time is preferably 2 to 100 hours, and more preferably 5 to 20 hours.

When a monomer having a hydroxy group is copolymerized, the hydroxy group may be substituted with an acetal group easily deprotected by an acid, such as an ethoxyethoxy group, for the polymerization, and the protected hydroxy group may be deprotected by a weak acid and water after the polymerization. Alternatively, the hydroxy group may be substituted with an acetyl group, a formyl group, a pivaloyl group, etc. and hydrolyzed with an alkali after the polymerization.

When hydroxystyrene or hydroxyvinylnaphthalene is copolymerized, acetoxystyrene or acetoxyvinylnaphthalene may be used instead of hydroxystyrene or hydroxyvinylnaphthalene, and the acetoxy group is deprotected by alkaline hydrolysis after the polymerization to be converted into hydroxystyrene or hydroxyvinylnaphthalene.

As a base in the alkaline hydrolysis, aqueous ammonia, triethylamine, etc. can be used. The reaction temperature is preferably −20 to 100° C., and more preferably 0 to 60° C. The reaction time is preferably 0.2 to 100 hours, and more preferably 0.5 to 20 hours.

The base polymer preferably has a weight-average molecular weight (Mw) of 1,000 to 500,000, and more preferably 2,000 to 30,000. The Mw is in terms of polystyrene by gel permeation chromatography (GPC) using THF as a solvent. Mw within the above range yields good heat resistance of the resist film and solubility in an alkaline developer.

When the base polymer has sufficiently narrow molecular weight distribution (Mw/Mn), a low molecular-weight and high molecular-weight polymers are absent, and thereby there is no risk of foreign matter observed on a pattern and deterioration in a pattern shape after the exposure. Since Mw and Mw/Mn have a larger effect as the pattern rule becomes smaller, the Mw/Mn of the base polymer is preferably 1.0 to 2.0 and particularly preferably 1.0 to 1.5, which indicates narrow distribution, in order to obtain the resist material suitably used for a fine pattern size.

The base polymer may contain two or more kinds of polymers having different composition ratios, Mw, and Mw/Mn.

Acid Generator

The inventive resist material may contain an acid generator to generate a strong acid (hereinafter, also referred to as the additive-type acid generator). The strong acid herein means: a compound having sufficient acidity for causing the deprotection reaction of the acid-labile group in the base polymer in a case of a chemically amplified positive-type resist material; or a compound having sufficient acidity for causing a polarity-changing reaction or crosslinking reaction with the acid in a case of a chemically amplified negative-type resist material. Containing such an acid generator enables the aforementioned sulfonium salt A to function as a quencher, and enables the inventive resist material to function as a chemically amplified positive-type resist material or a chemically amplified negative-type resist material.

Examples of the acid generator include a compound to generate an acid by sensitizing with active ray or radiation (photoacid generator). The photoacid generator may be any compound that generates an acid by high-energy ray irradiation, but the photoacid generator preferably generates a sulfonic acid, an imide acid, or a methide acid. Examples of preferable photoacid generators include sulfonium salts, iodonium salts, sulfonyldiazomethanes, N-sulfonyloxyimides, and oxime-O-sulfonate-type acid generators. Specific examples of the photoacid generator include those described in paragraphs [0122] to [0142] of JP 2008-111103 A.

As the photoacid generator, a sulfonium salt represented by the following general formula (3-1) and an iodonium salt represented by the following general formula (3-2) can also be preferably used.

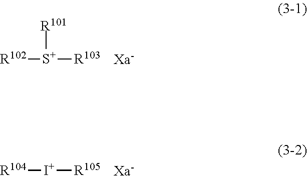

In the formulae (3-1) and (3-2), R¹⁰¹ to R¹⁰⁵ each independently represent a halogen atom or a hydrocarbyl group having 1 to 25, preferably 1 to 20, carbon atoms and optionally having a heteroatom. The hydrocarbyl group may be a saturated or unsaturated group, and may be any one of linear, branched, and cyclic groups. Specific examples thereof include groups same as those exemplified as the hydrocarbyl group represented by R² to R⁴ in description of the formulae (1) and (1′).

R¹⁰¹ and R¹⁰² are optionally bonded each other to form a ring together with the sulfur atom to which these groups are bonded. Examples of the ring in this case include rings same as those exemplified as the rings that can be formed by bonding R² and R³ together with the sulfur atom to which these groups are bonded, described in the general formulae (1) and (1′).

Examples of the cation of the sulfonium salt represented by the general formula (3-1) include cations same as those exemplified as the cation of the sulfonium salt represented by the general formulae (1) and (1′).

Examples of the cation of the iodonium salt represented by the general formula (3-2) include the following cations, but the cation is not limited thereto.

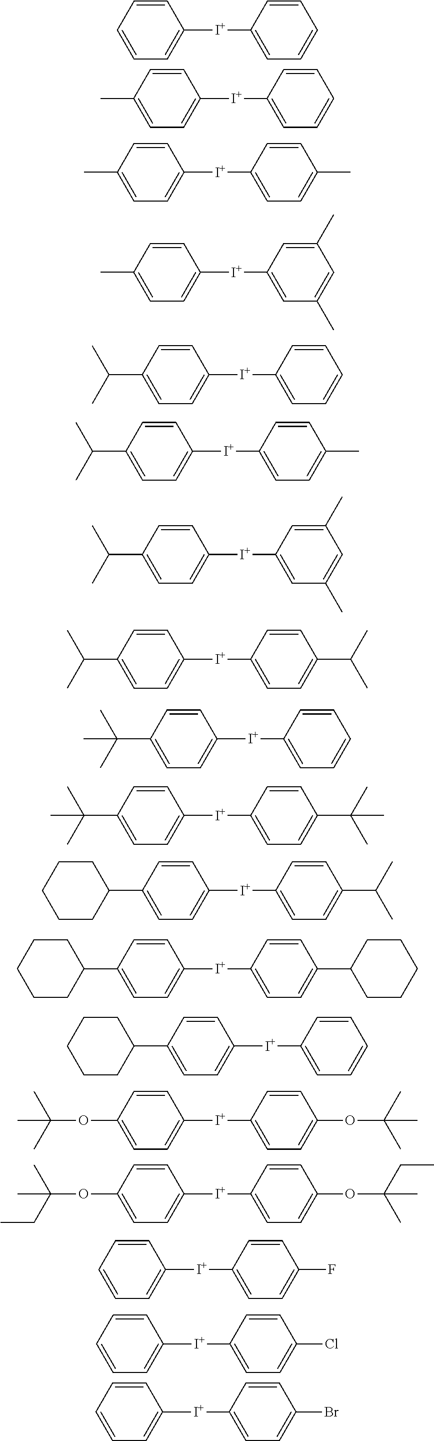

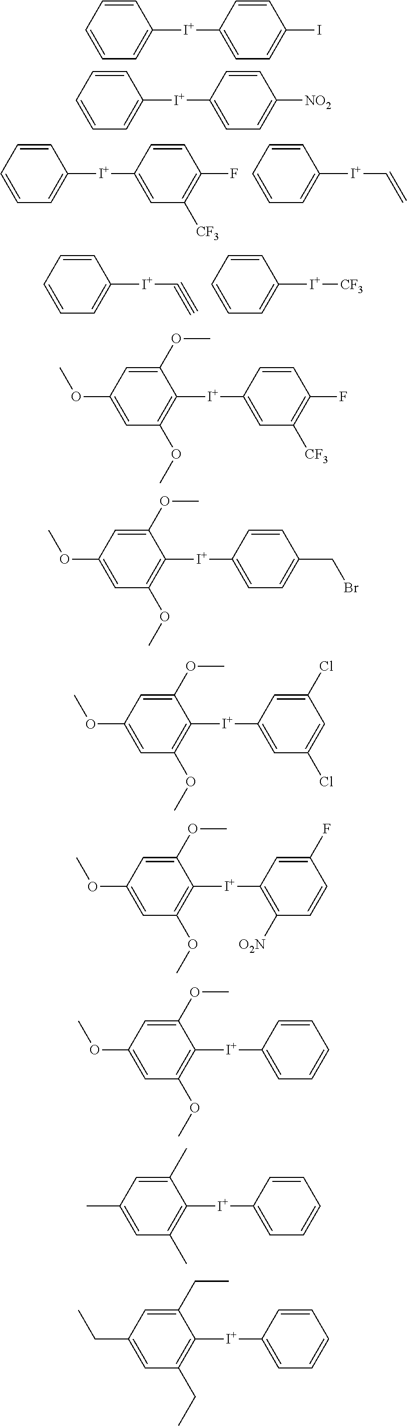

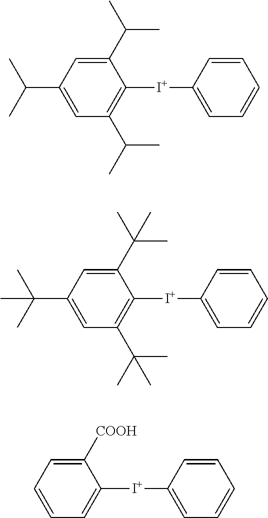

In the general formulae (3-1) and (3-2), Xa⁻ represents an anion selected from the following general formulae (3A) to (3D).

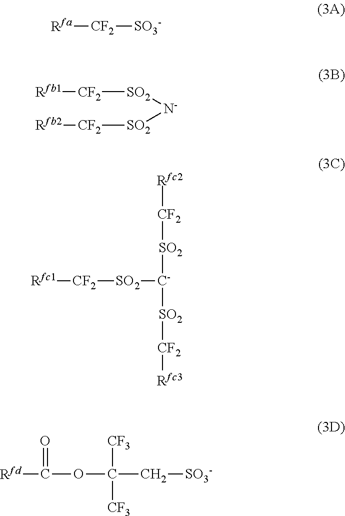

In the general formula (3A), R^{fa} represents a fluorine atom or a hydrocarbyl group having 1 to 40 carbon atoms and optionally having a heteroatom. The hydrocarbyl group may be a saturated or unsaturated group, and may be any of linear, branched, and cyclic groups. Specific examples thereof include groups same as those exemplified as a hydrocarbyl group represented by R¹¹¹ in the formula (3A′), described later.

The anion represented by the general formula (3A) is preferably represented by the following general formula (3A′).

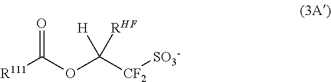

In the general formula (3A′), R^{HF} represents a hydrogen atom or a trifluoromethyl group, and preferably a trifluoromethyl group. R¹¹¹ represents a hydrocarbyl group having 1 to 38 carbon atoms and optionally having a heteroatom. The heteroatom is preferably an oxygen atom, a nitrogen atom, a sulfur atom, a halogen atom, etc., and more preferably an oxygen atom. The hydrocarbyl group particularly preferably has 6 to 30 carbon atoms in terms of obtaining high resolution in fine patterning.

The hydrocarbyl group represented by R¹¹¹ may be a saturated or unsaturated group, and may be any of linear, branched, and cyclic groups. Specific examples thereof include: alkyl groups having 1 to 38 carbon atoms, such as a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosanyl group; cyclic saturated hydrocarbyl groups having 3 to 38 carbon atoms, such as a cyclopentyl group, a cyclohexyl group, a 1-adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group; unsaturated aliphatic hydrocarbyl groups having 2 to 38 carbon atoms, such as an allyl group and a 3-cyclohexenyl group; aryl groups having 6 to 38 carbon atoms, such as a phenyl group, a 1-naphthyl group, and a 2-naphthyl group; aralkyl groups having 7 to 38 carbon atoms, such as a benzyl group and a diphenylmethyl group; and groups obtained by combining these groups.

A part or all of hydrogen atoms in these groups are optionally substituted with a group having a heteroatom such as an oxygen atom, a sulfur atom, a nitrogen atom, and a halogen atom. A part of carbon atoms in these groups is optionally substituted with a group having a heteroatom such as an oxygen atom, a sulfur atom, and a nitrogen atom. As a result, optionally contained are a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride, a haloalkyl group, etc. Examples of the hydrocarbyl group having a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group.

Synthesis of the sulfonium salt having the anion represented by the general formula (3A′) is detailed in JP 2007-145797 A, JP 2008-106045 A, JP 2009-7327 A, and JP 2009-258695 A. Sulfonium salts described in JP 2010-215608 A, JP 2012-41320 A, JP 2012-106986 A, JP 2012-153644 A, etc. are also preferably used.

Examples of the anion represented by the general formula (3A) include anions same as those exemplified as the anion represented by the formula (1A) of JP 2018-197853 A.

In the general formula (3B), R^{fb1} and R^{fb2} each independently represent a fluorine atom or a hydrocarbyl group having 1 to 40 carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R¹¹¹ in the general formula (3A′). R^{fb1} and R^{fb2} preferably represent a fluorine atom or a linear fluorinated alkyl group having 1 to 4 carbon atoms. R^{fb1} and R^{fb2} are optionally bonded to each other to form a ring together with the group to which these groups are bonded (—CF₂—SO₂—N⁻—SO₂—CF₂—), and the group obtained in this case by bonding R^{fb1} and R^{fb2} each other is preferably a fluorinated ethylene group or a fluorinated propylene group.

In the general formula (3C), R^{fc1}, R^{fc2}, and R^{fc3} each independently represent a fluorine atom or a hydrocarbyl group having 1 to 40 carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R¹¹¹ in the general formula (3A′). R^{fc1}, R^{fc2}, and R^{fc3} preferably represent a fluorine atom or a linear fluorinated alkyl group having 1 to 4 carbon atoms. R^{fc1} and R^{fc2} are optionally bonded to each other to form a ring together with the group to which these groups are bonded (—CF₂—SO₂—C⁻—SO₂—CF₂—), and the group obtained in this case by bonding R^{fc1} and R^{fc2} each other is preferably a fluorinated ethylene group or a fluorinated propylene group.

In the general formula (3D), R^{fd} represents a hydrocarbyl group having 1 to 40 carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R¹¹¹ in the general formula (3A′).

Synthesis of the sulfonium salt having the anion represented by the general formula (3D) is detailed in JP 2010-215608 A and JP 2014-133723 A.

Examples of the anion represented by the general formula (3D) include anions same as those exemplified as the anions represented by the formula (1D) in JP 2018-197853 A.

The photoacid generator having the anion represented by the general formula (3D) has no fluorine atom at the α-position of the sulfo group, but has two trifluoromethyl groups at the β-position, resulting in the photoacid generator having sufficient acidity for cleaving the acid-labile group in the base polymer. Therefore, it can be used as a photoacid generator.

As the photoacid generator, a compound represented by the following general formula (4) can also be preferably used.

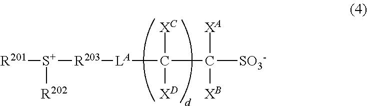

In the general formula (4), R²⁰¹ and R²⁰² each independently represent a halogen atom or a hydrocarbyl group having 1 to 30 carbon atoms and optionally having a heteroatom. R²⁰³ represents a hydrocarbylene group having 1 to 30 carbon atoms and optionally having a heteroatom. Any two of R²⁰¹, R²⁰² and R²⁰³ are optionally bonded to each other to form a ring together with the sulfur atom to which these groups are bonded. In this case, examples of the ring include rings same as those exemplified as the rings that can be formed by bonding R² and R³ together with the sulfur atom to which these groups are bonded, described in the general formulae (1) and (1′).

The hydrocarbyl group represented by R²⁰¹ and R²⁰² may be a saturated or unsaturated group, and may be any of linear, branched, and cyclic groups. Specific examples thereof include: alkyl groups having 1 to 30 carbon atoms, such as a methyl group, an ethyl group, a propyl group, an isopropyl group, an n-butyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 30 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decanyl group, and an adamantyl group; aryl groups having 6 to 30 carbon atoms, such as a phenyl group, a methylphenyl group, an ethylphenyl group, an n-propylphenyl group, an isopropylphenyl group, an n-butylphenyl group, an isobutylphenyl group, a sec-butylphenyl group, a tert-butylphenyl group, a naphthyl group, a methylnaphthyl group, an ethylnaphthyl group, an n-propylnaphthyl group, an isopropylnaphthyl group, an n-butylnaphthyl group, an isobutylnaphthyl group, a sec-butylnaphthyl group, a tert-butylnaphthyl group, and an anthracenyl group; and groups obtained by combining these groups. A part or all of hydrogen atoms in these groups are optionally substituted with a group having a heteroatom such as an oxygen atom, a sulfur atom, a nitrogen atom, and a halogen atom. A part of carbon atoms in these groups is optionally substituted with a group having a heteroatom such as an oxygen atom, a sulfur atom, and a nitrogen atom. As a result, optionally contained are a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride, a haloalkyl group, etc.

The hydrocarbylene group represented by R²⁰³ may be a saturated or unsaturated group, and may be any of linear, branched, and cyclic groups. Specific examples thereof include: alkanediyl groups having 1 to 30 carbon atoms, such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; cyclic saturated hydrocarbylene groups having 3 to 30 carbon atoms, such as a cyclopentanediyl group, a cyclohexanediyl group, a norbornanediyl group, and an adamantanediyl group; arylene groups having 6 to 30 carbon atoms, such as a phenylene group, a methylphenylene group, an ethylphenylene group, an n-propylphenylene group, an isopropylphenylene group, an n-butylphenylene group, an isobutylphenylene group, a sec-butylphenylene group, a tert-butylphenylene group, a naphthylene group, a methylnaphthylene group, an ethylnaphthylene group, an n-propylnaphthylene group, an isopropylnaphthylene group, an n-butylnaphthylene group, an isobutylnaphthylene group, a sec-butylnaphthylene group, and a tert-butylnaphthylene group; and groups obtained by combining these groups. A part or all of hydrogen atoms in these groups are optionally substituted with a group having a heteroatom such as an oxygen atom, a sulfur atom, a nitrogen atom, and a halogen atom. A part of carbon atoms in these groups is optionally substituted with a group having a heteroatom such as an oxygen atom, a sulfur atom, and a nitrogen atom. As a result, optionally contained are a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride, a haloalkyl group, etc. The heteroatom is preferably an oxygen atom.

In the general formula (4), L^{A} represents a single bond, an ether bond, or a hydrocarbylene group having 1 to 20 carbon atoms and optionally having a heteroatom. The hydrocarbylene group may be a saturated or unsaturated group, and may be any of linear, branched, and cyclic groups. Specific examples thereof include groups same as those exemplified as the hydrocarbylene group represented by R²⁰³.

In the general formula (4), X^{A}, X^{B}, X^{C}, and X^{D} each independently represent a hydrogen atom, a fluorine atom, or a trifluoromethyl group. Note that, at least one of X^{A}, X^{B}, X^{C}, and X^{D} represents a fluorine atom or a trifluoromethyl group.

In the general formula (4), “d” represents an integer of 0 to 3.

The photoacid generator represented by the general formula (4) is preferably represented by the following general formula (4′).

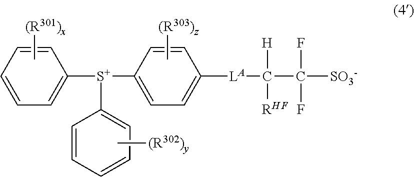

In the general formula (4′), L A represents the same as above. R^{HF} represents a hydrogen atom or a trifluoromethyl group, and preferably a trifluoromethyl group. R³⁰¹, R³⁰², and R³⁰³ each independently represent a hydrogen atom or a hydrocarbyl group having 1 to 20 carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R¹¹¹ in the formula (3A′). “x” and “y” each independently represent an integer of 0 to 5, and “z” represents an integer of 0 to 4.

Examples of the photoacid generator represented by the general formula (4) include photoacid generators same as those exemplified as photoacid generators represented by the formula (2) in JP 2017-026980 A.

Among the above photoacid generators, the photoacid generators having the anion represented by the general formula (3A′) or (3D) are particularly preferable since having small acid diffusion and excellent solubility in the solvent. The photoacid generators represented by the formula (4′) are particularly preferable since having extremely small acid diffusion.

As the photoacid generator, a sulfonium salt or iodonium salt having an anion having an aromatic ring substituted with an iodine atom or a bromine atom can also be used. Examples of such a salt include salts represented by the following general formula (5-1) or (5-2).

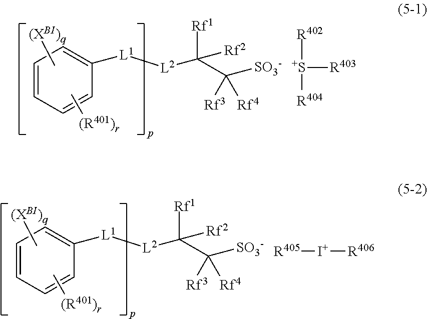

In the general formulae (5-1) and (5-2), “p” represents an integer satisfying 1≤p≤3. “q” and “r” represent an integer satisfying 1≤q≤5, 0≤r≤3, and 1≤q+r≤5. “q” preferably represents an integer satisfying 1≤q≤3, and more preferably 2 or 3. “r” preferably represents an integer satisfying 0≤r≤2.

In the general formulae (5-1) and (5-2), X^{BI} represents an iodine atom or a bromine atom. When “p” and/or “q” represent 2 or more, X^{BI} may be same as or different from each other.

In the general formulae (5-1) and (5-2), L¹ represents a single bond, an ether bond, an ester bond, or a saturated hydrocarbylene group having 1 to 6 carbon atoms and optionally having an ether bond or an ester bond. The saturated hydrocarbylene group may be any of linear, branched, and cyclic groups.

In the general formulae (5-1) and (5-2), L² represents a single bond or a divalent linking group having 1 to 20 carbon atoms when “p” represents 1, and L² represents a (p+1)-valent linking group having 1 to 20 carbon atoms when “p” represents 2 or 3. The linking group optionally has an oxygen atom, a sulfur atom, a halogen atom, or a nitrogen atom.

In the general formulae (5-1) and (5-2), R⁴⁰¹ represents a hydroxy group, a carboxy group, a fluorine atom, a chlorine atom, a bromine atom, an amino group, a hydrocarbyl group having 1 to 20 carbon atoms, a hydrocarbyloxy group having 1 to 20 carbon atoms, a hydrocarbylcarbonyl group having 2 to 20 carbon atoms, a hydrocarbyloxycarbonyl group having 2 to 20 carbon atoms, a hydrocarbylcarbonyloxy group having 2 to 20 carbon atoms, a hydrocarbylsulfonyloxy group having 1 to 20 carbon atoms, —N(R^{401A})(R^{401B}), —N(R^{401C})—C(═O)—R^{401D}, or —N(R^{401C})—C(═O)—O—R^{401D}. The hydrocarbyl group, the hydrocarbyloxy group, hydrocarbylcarbonyl group, the hydrocarbyloxycarbonyl group, the hydrocarbylcarbonyloxy group, and the hydrocarbylsulfonyloxy group optionally have a fluorine atom, a chlorine atom, a bromine atom, a hydroxy group, an amino group, an ether bond, an ester bond, or an amide bond. R^{401A} and R^{401B} each independently represent a hydrogen atom or a saturated hydrocarbyl group having 1 to 6 carbon atoms. R^{401C} represents a hydrogen atom or a saturated hydrocarbyl group having 1 to 6 carbon atoms, and optionally having a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{401D} represents an aliphatic hydrocarbyl group having 1 to 16 carbon atoms, an aryl group having 6 to 14 carbon atoms, or an aralkyl group having 7 to 15 carbon atoms, and optionally has a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be a saturated or unsaturated group, and may be any of linear, branched, and cyclic groups. The saturated hydrocarbyl group, the saturated hydrocarbyloxy group, the saturated hydrcarbyloxycarbonyl group, the saturated hydrocarbylcarbonyl group, and the saturated hydrocarbylcarbonyloxy group may be any of linear, branched, and cyclic groups. When “p” and/or “r” represent 2 or more, each R⁴⁰¹ may be same as or different from each other.

Among these, R⁴⁰¹ preferably represents a hydroxy group, —N(R^{401C})—C(═O)—R^{401D}, —N(R^{401C})—C(═O)—O—R^{401D}, a fluorine atom, a chlorine atom, a bromine atom, a methyl group, a methoxy group, etc.

In the general formulae (5-1) and (5-2), Rf¹ to Rf⁴ each independently represent a hydrogen atom, a fluorine atom, or a trifluoromethyl group, and at least one of them represents a fluorine atom or a trifluoromethyl group. Rf¹ and Rf² are optionally integrated to form a carbonyl group. In particular, both of Rf³ and Rf⁴ preferably represent fluorine atoms.

In the general formulae (5-1) and (5-2), R⁴⁰² to R⁴⁰⁶ each independently represent a halogen atom or a hydrocarbyl group having 1 to 20 carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R² to R³ in the description of the general formulae (1) and (1′). A part or all of hydrogen atoms in these groups are optionally substituted with a hydroxy group, a carboxy group, a halogen atom, a cyano group, a nitro group, a mercapto group, a sultone ring, a sulfone group, or a sulfonium-salt-containing group. A part of carbon atoms in these groups is optionally substituted with an ether bond, an ester bond, a carbonyl group, an amide bond, a carbonate bond, or a sulfonate ester bond. Furthermore, R⁴⁰² and R⁴⁰³ are optionally bonded to each other to form a ring together with the sulfur atom to which these groups are bonded. In this case, examples of the ring include rings same as those exemplified as the ring that can be formed by bonding R² and R³ each other together with the sulfur atom to which these groups are bonded, described in the general formulae (1) and (1′).

Examples of the cation of the sulfonium salt represented by the general formula (5-1) include cations same as those exemplified as the cation of the sulfonium salt represented by the general formulae (1) and (1′). Examples of the cation of the iodonium salt represented by the general formula (5-2) include cations same as those exemplified as the cation of the iodonium salt represented by the general formula (3-2).

Examples of the anion of the onium salt represented by the general formula (5-1) or (5-2) include the following anions, but the anion is not limited thereto. In the following formulae, X^{BI} represents the same as above.

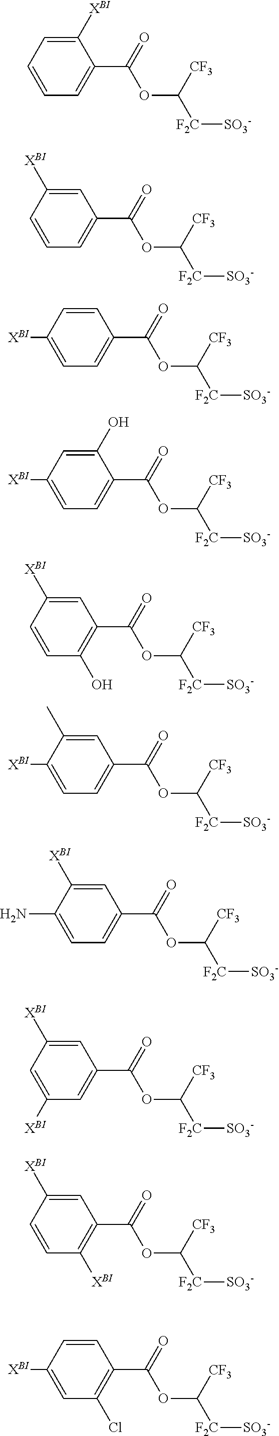

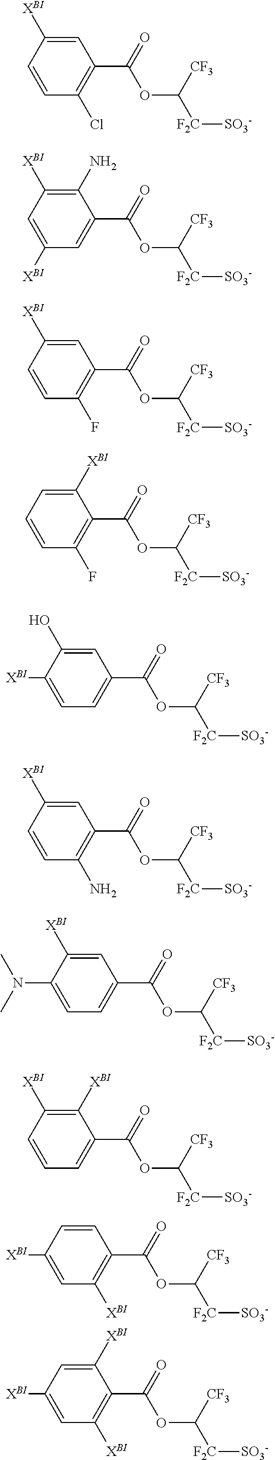

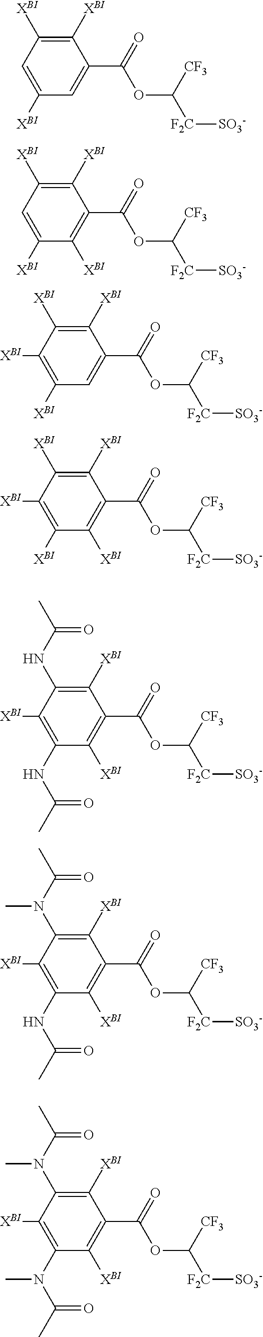

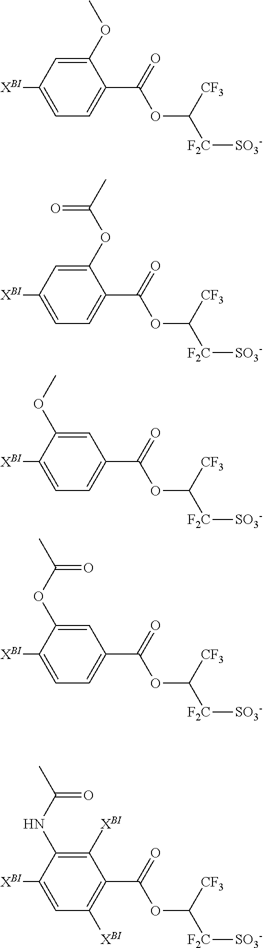

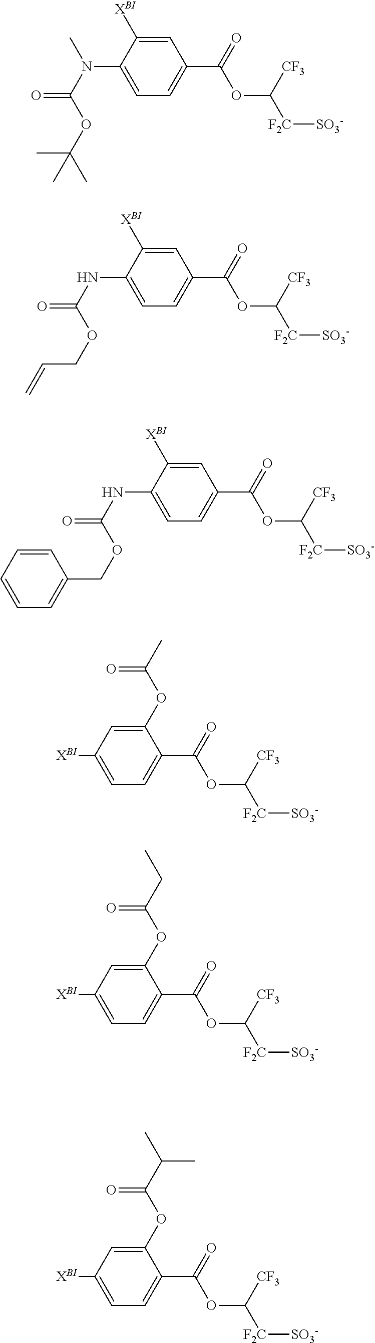

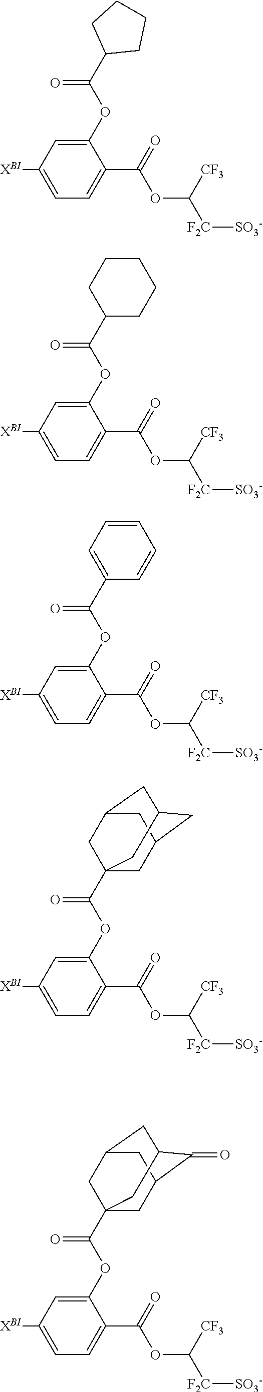

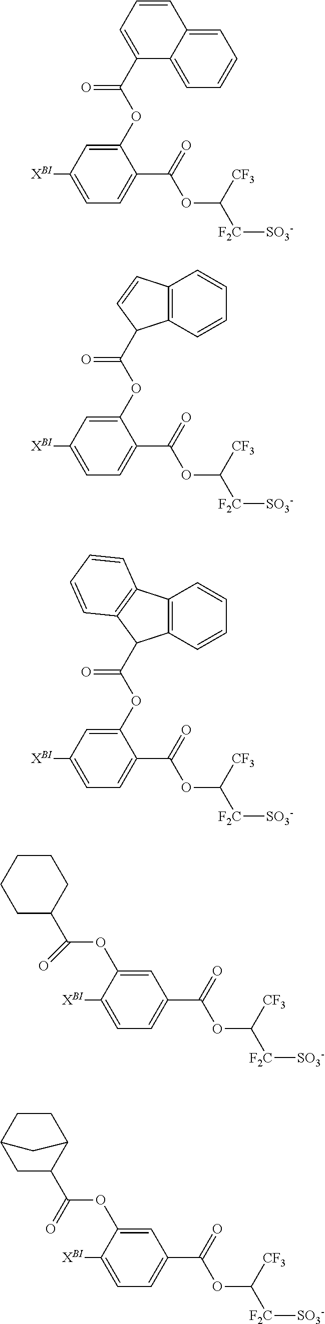

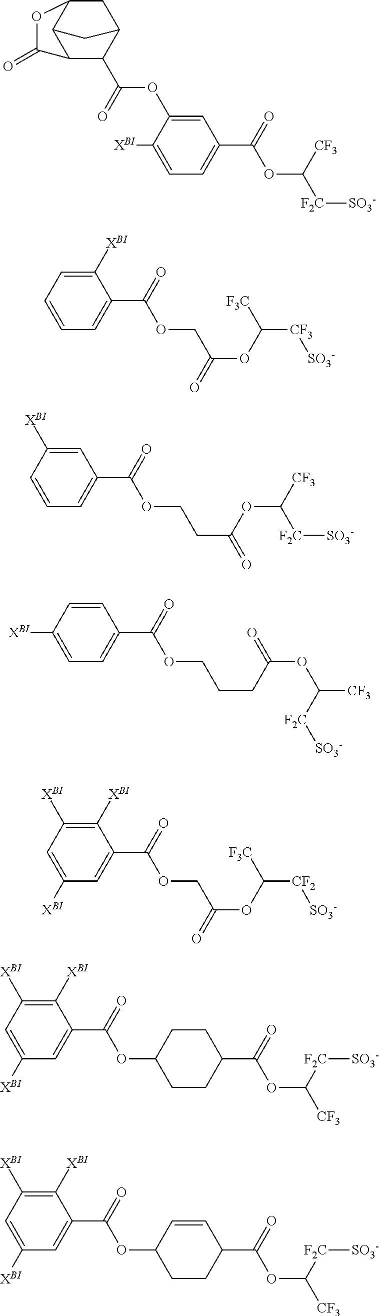

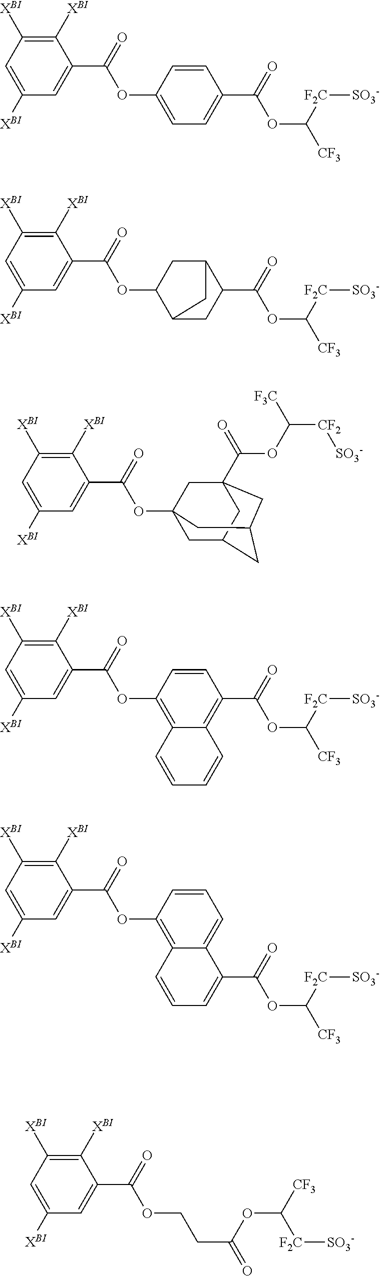

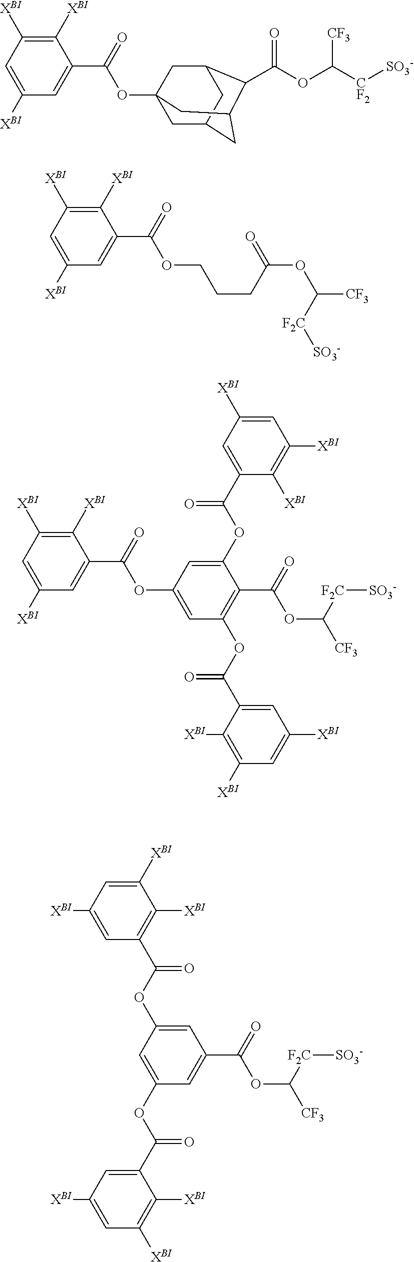

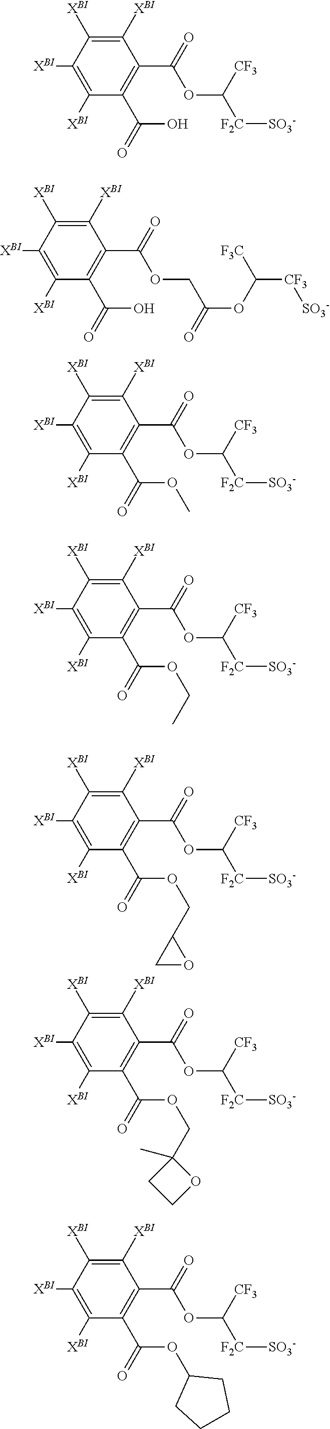

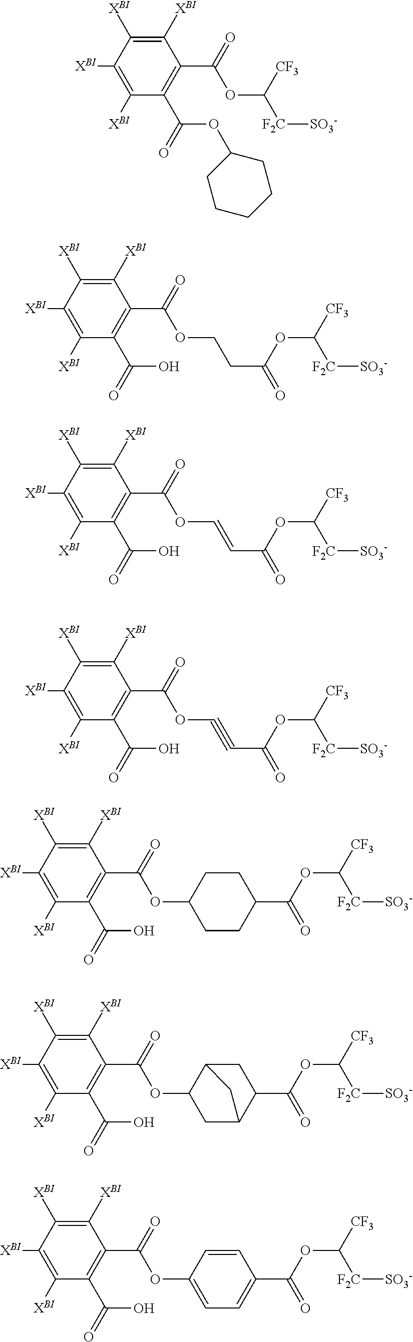

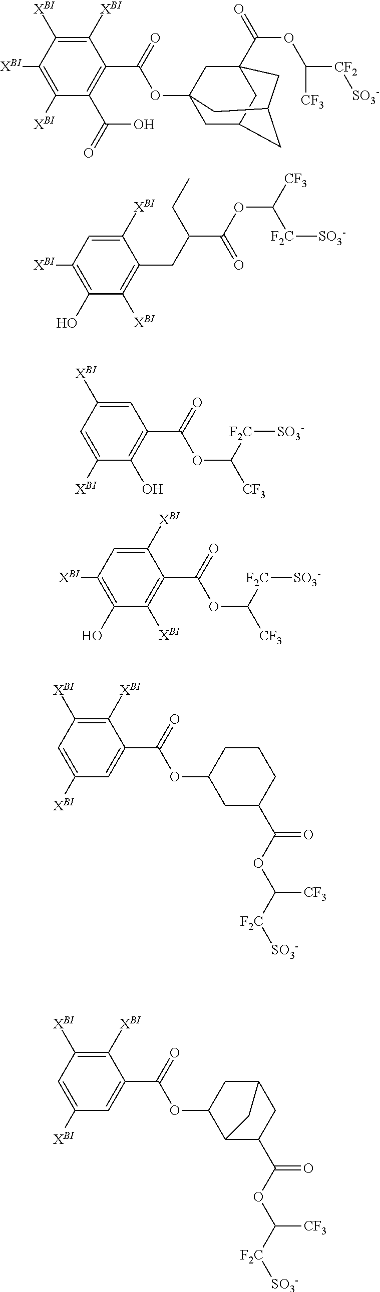

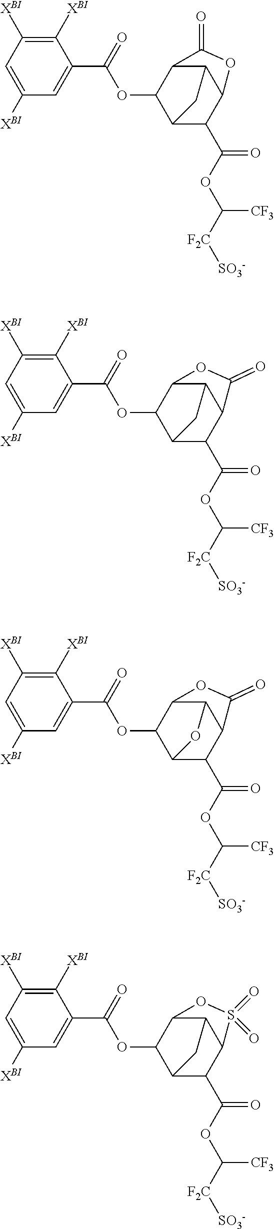

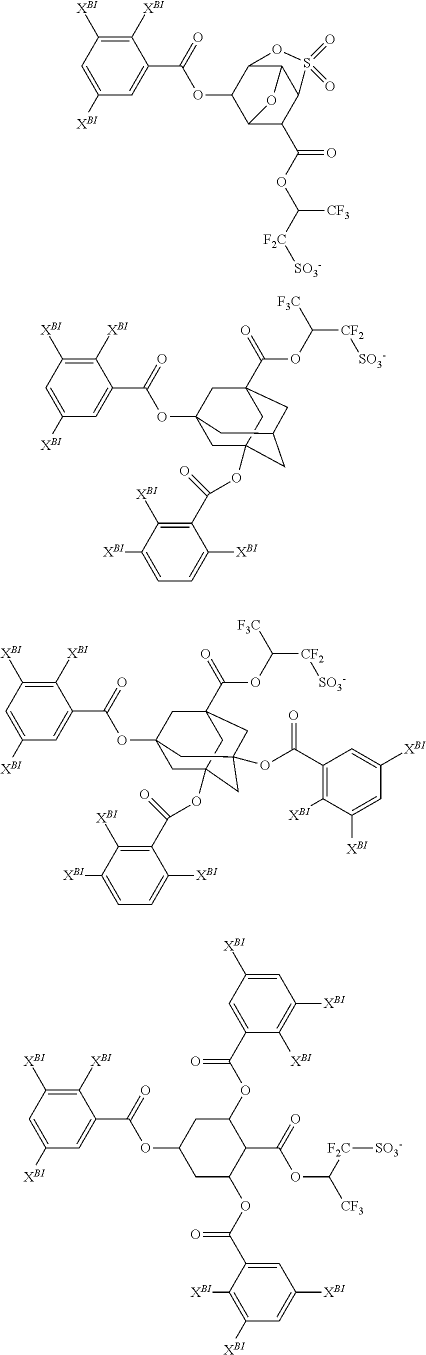

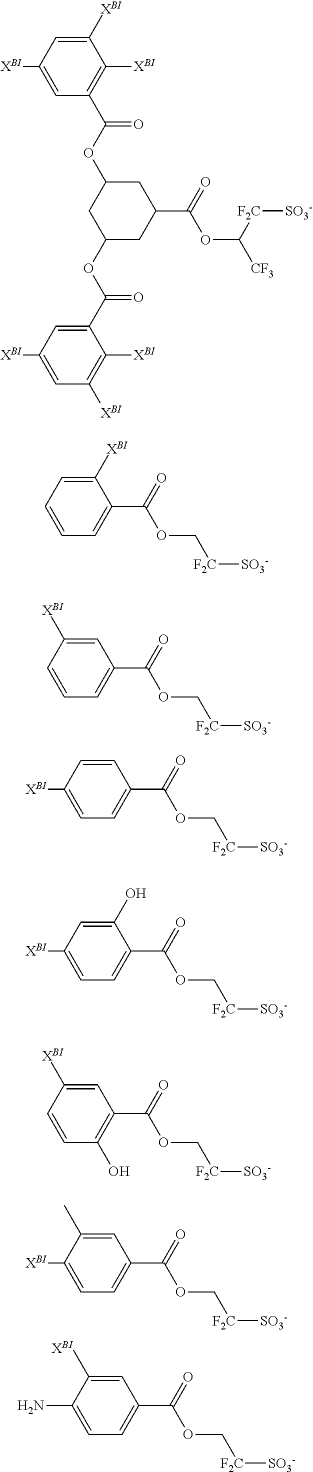

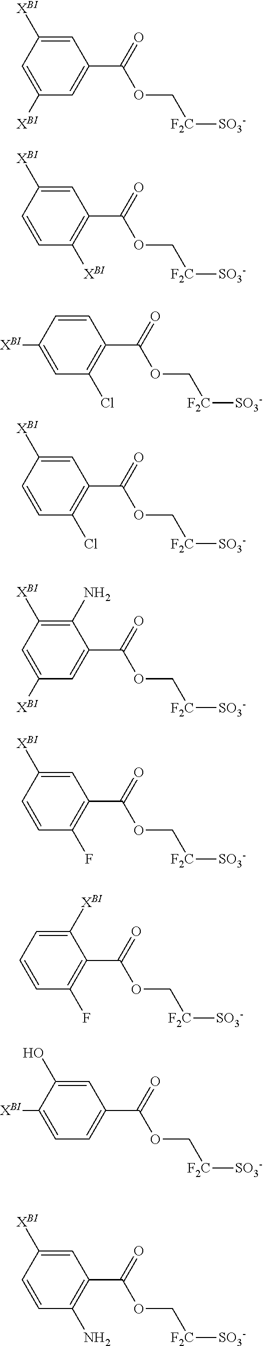

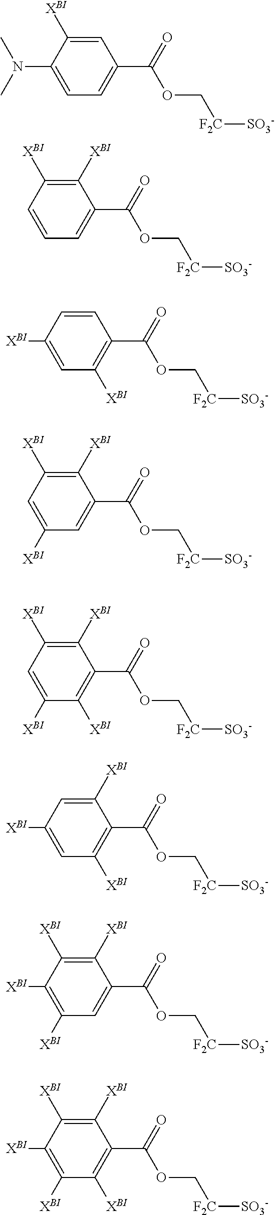

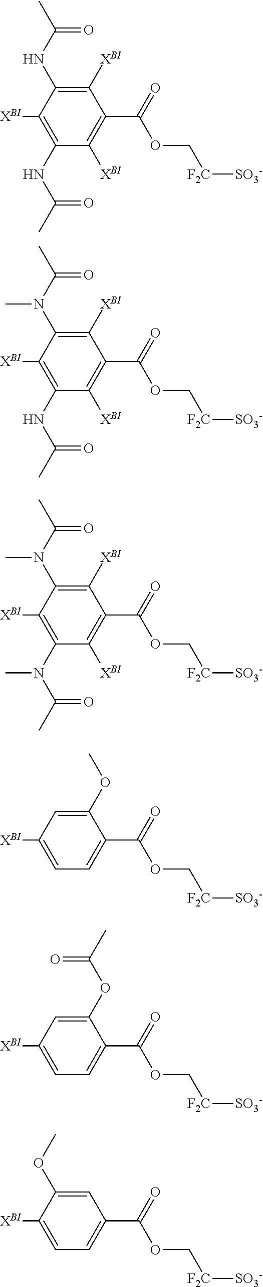

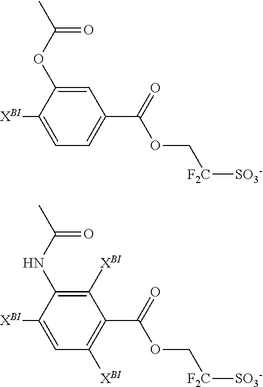

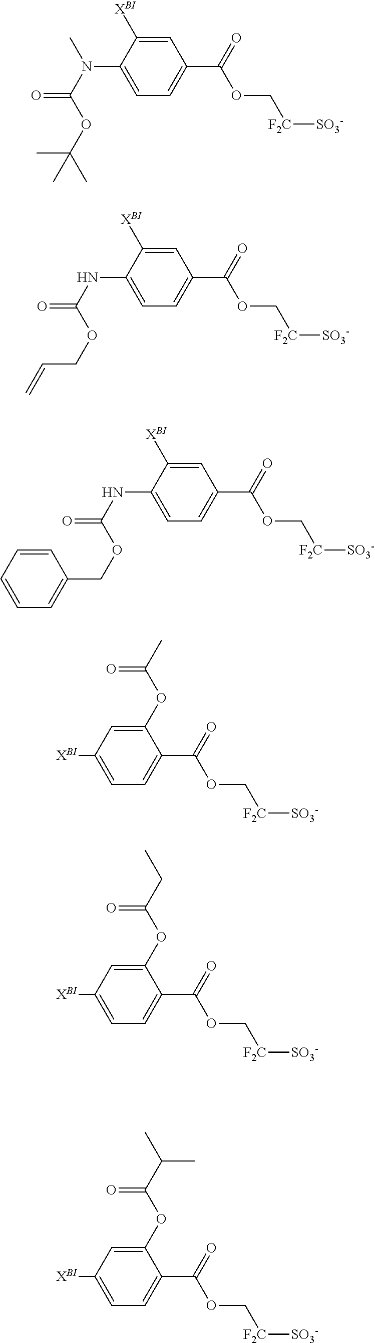

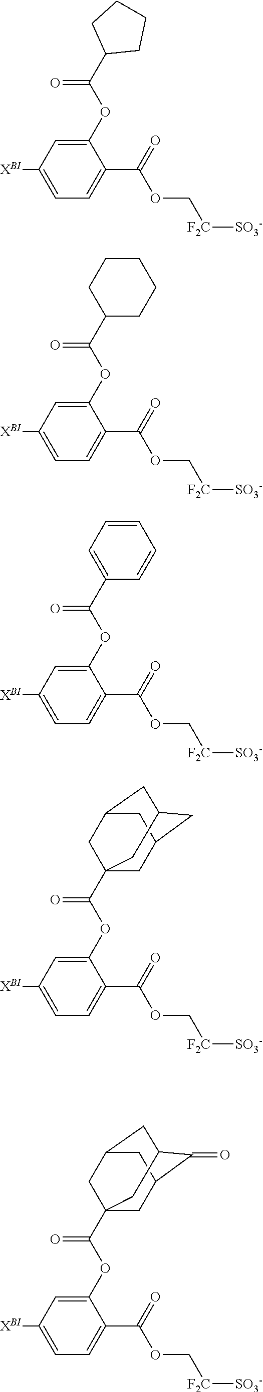

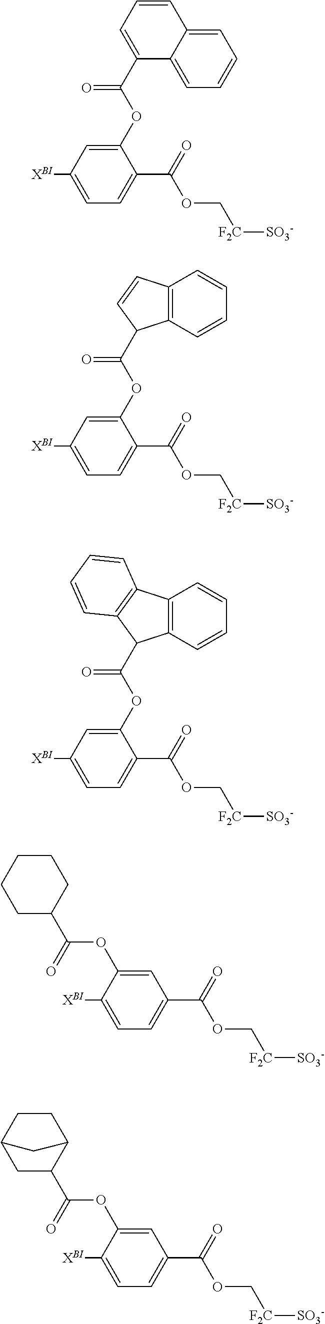

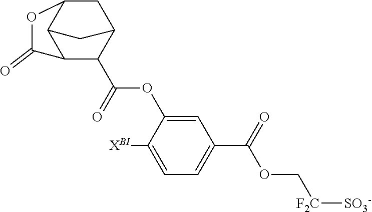

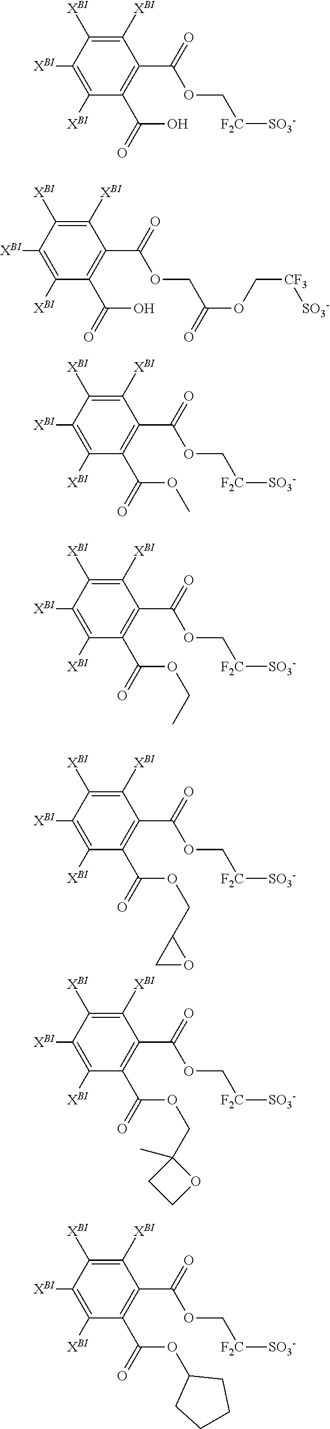

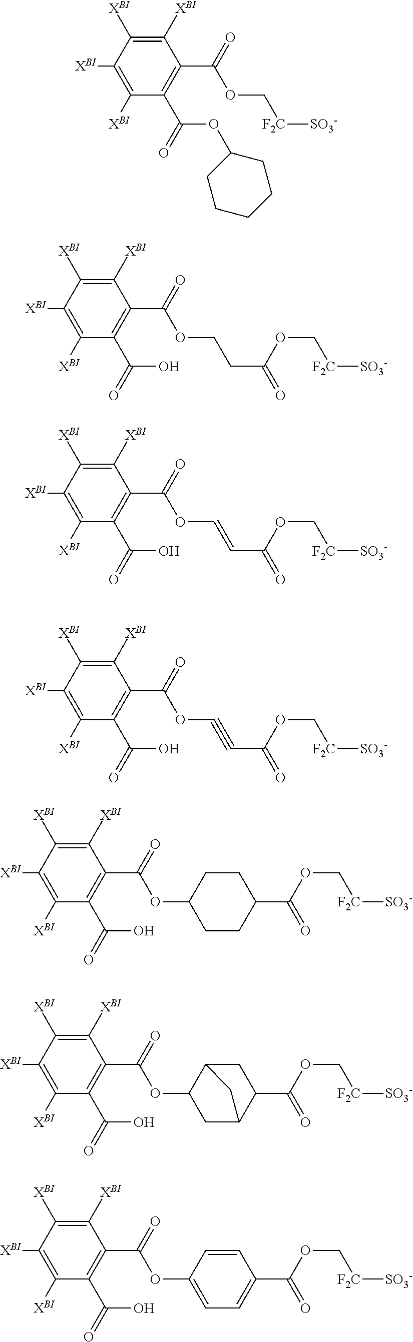

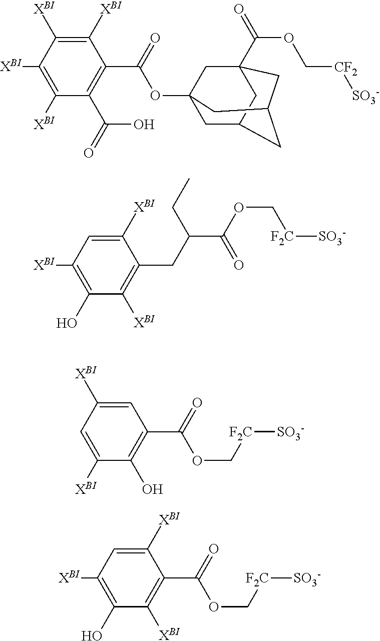

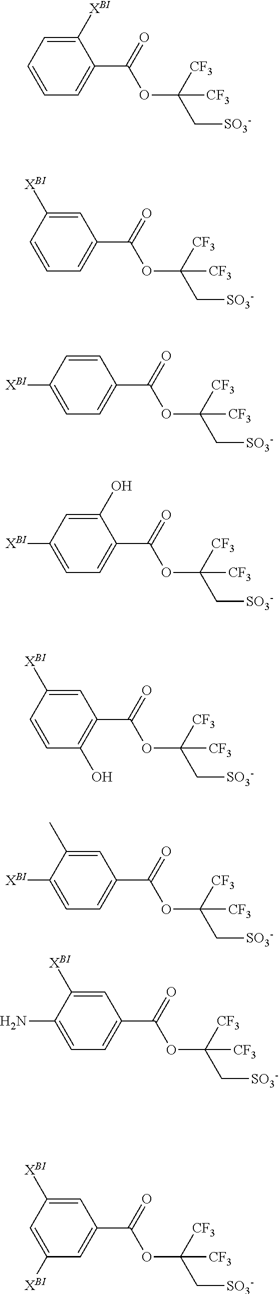

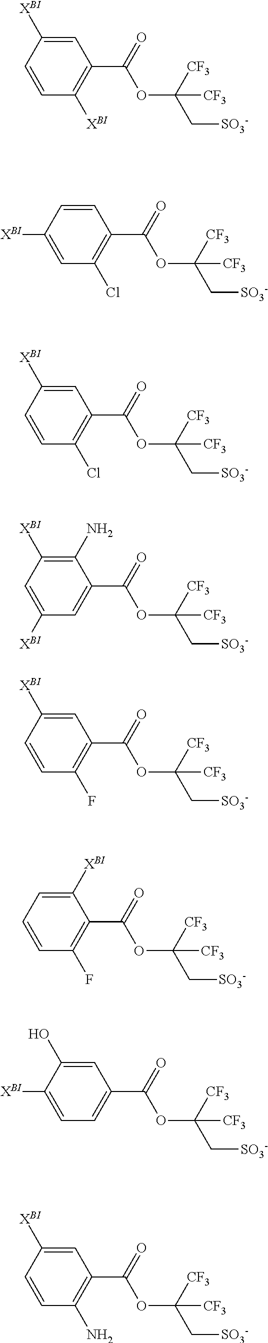

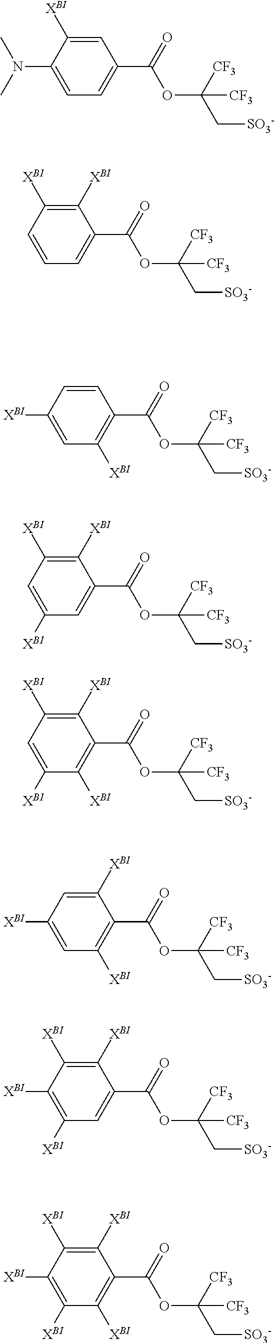

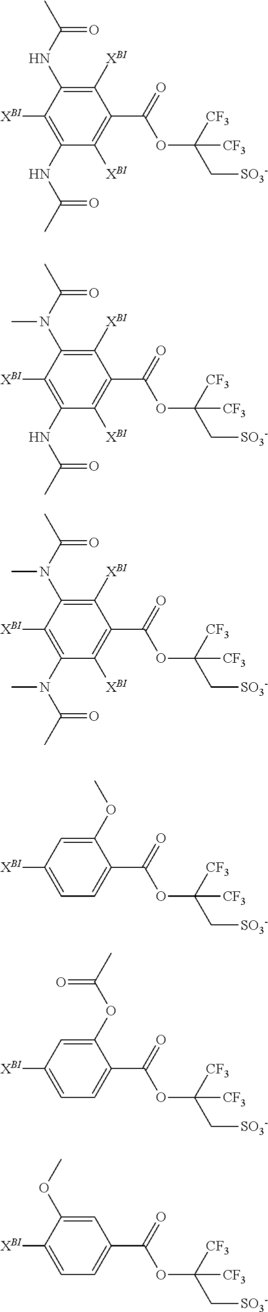

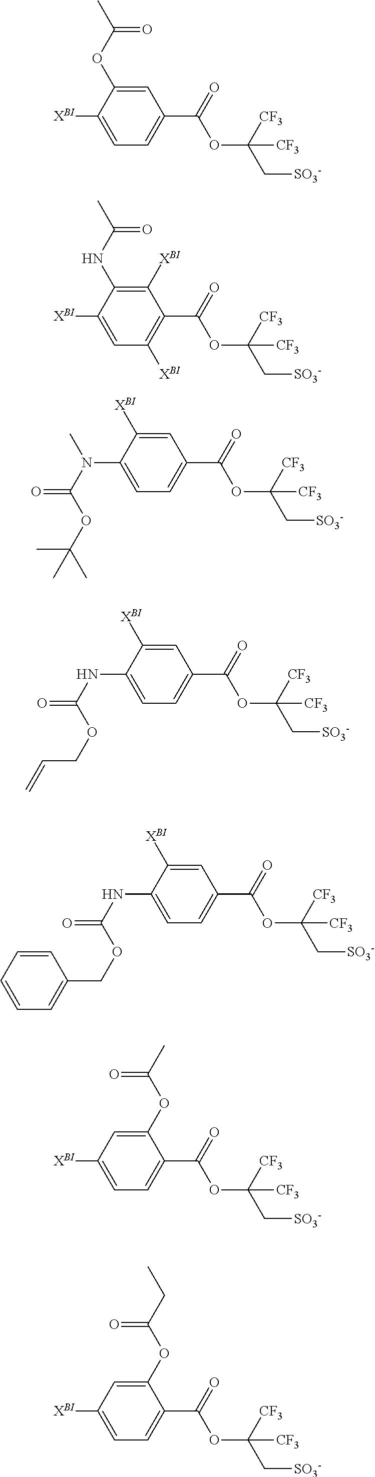

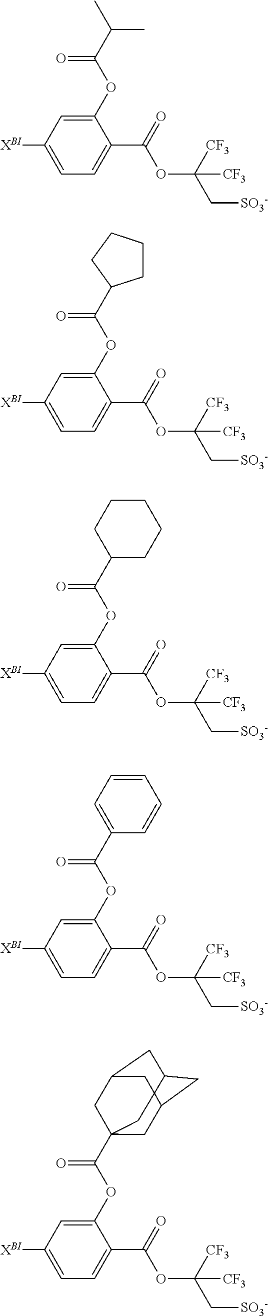

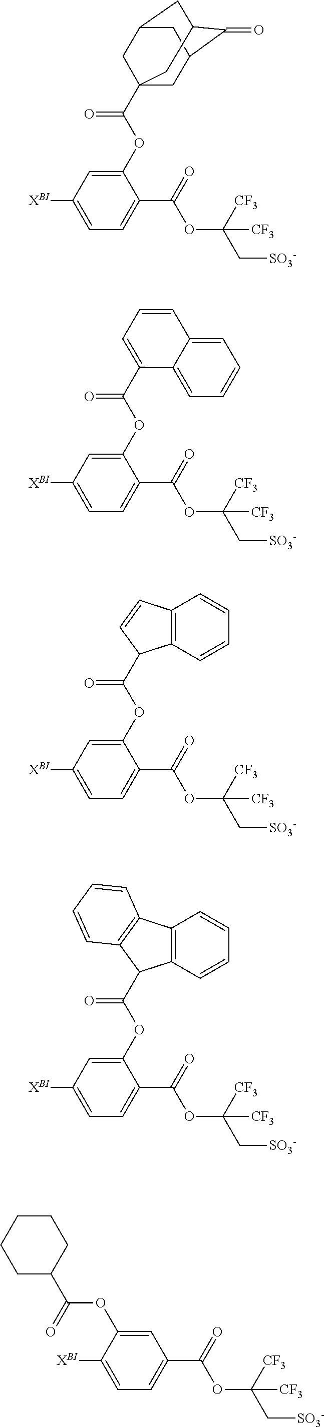

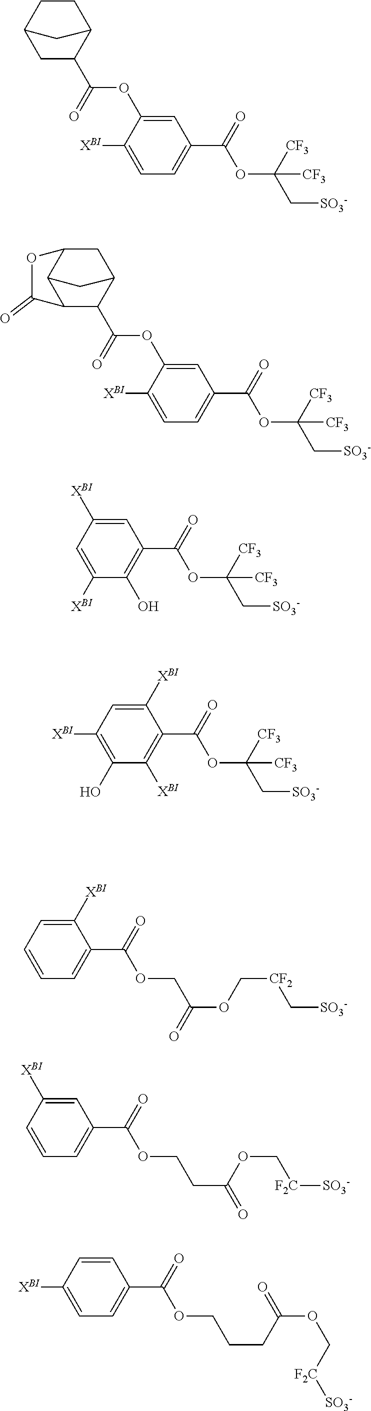

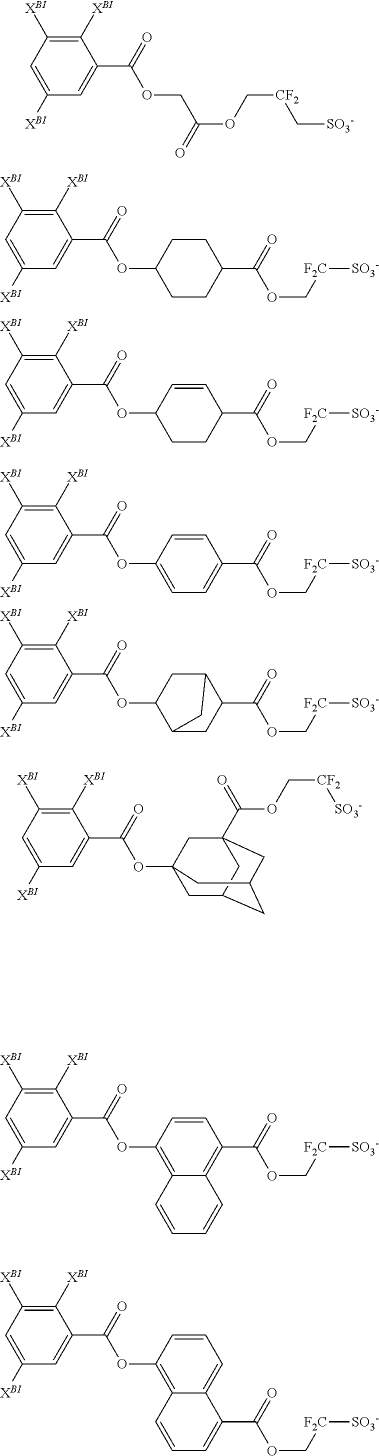

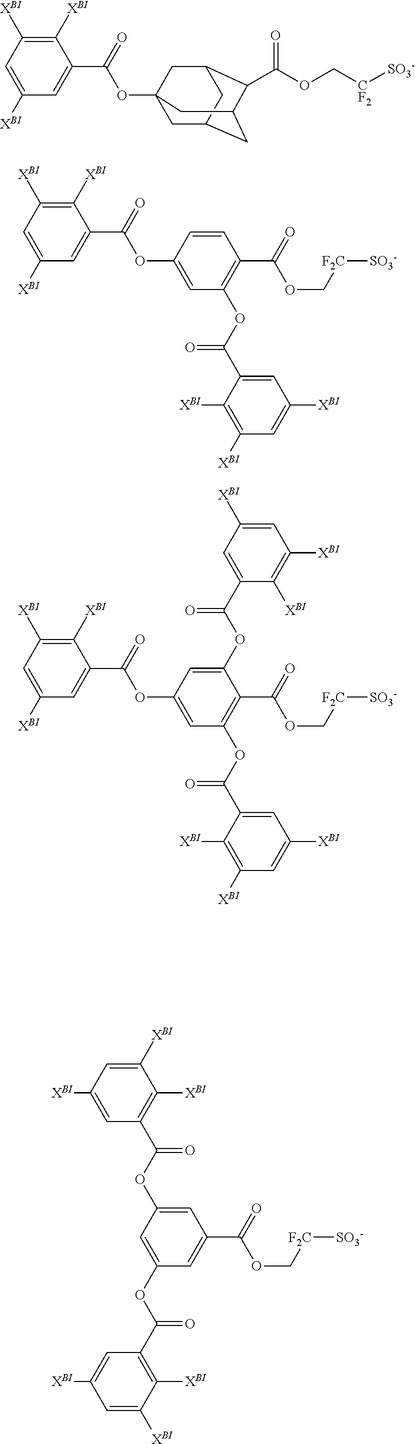

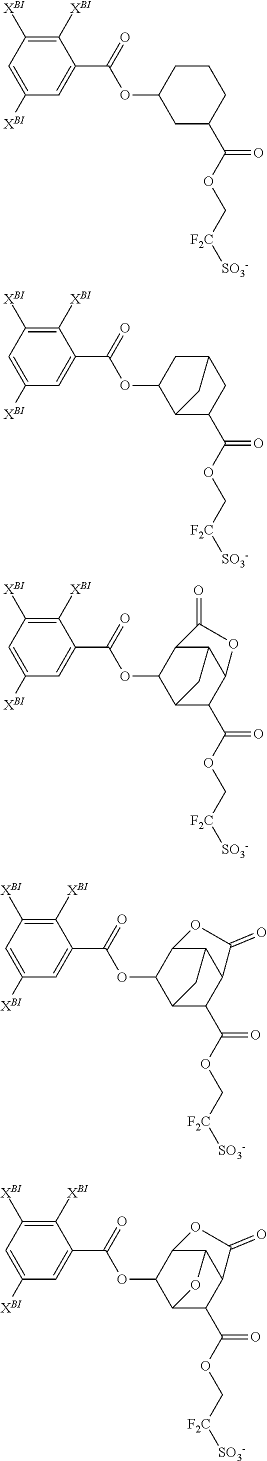

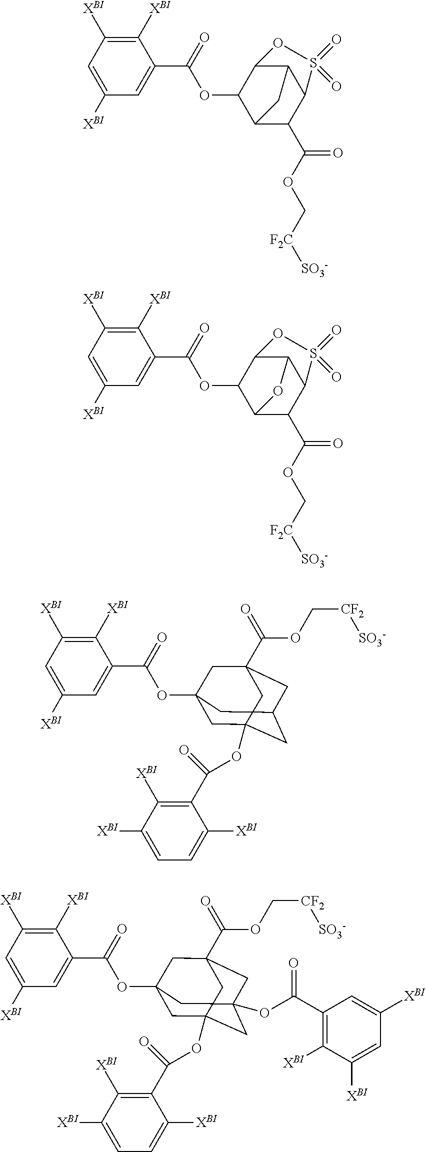

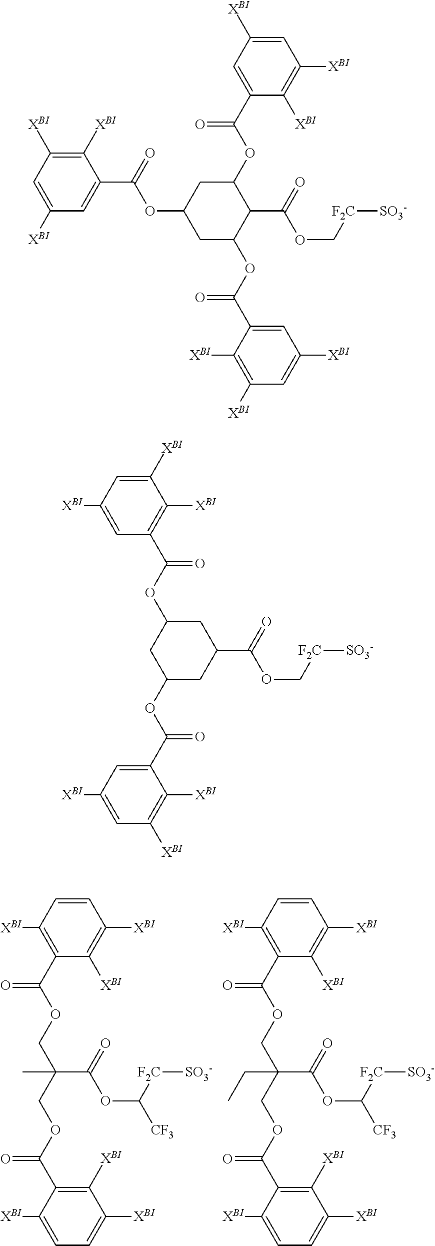

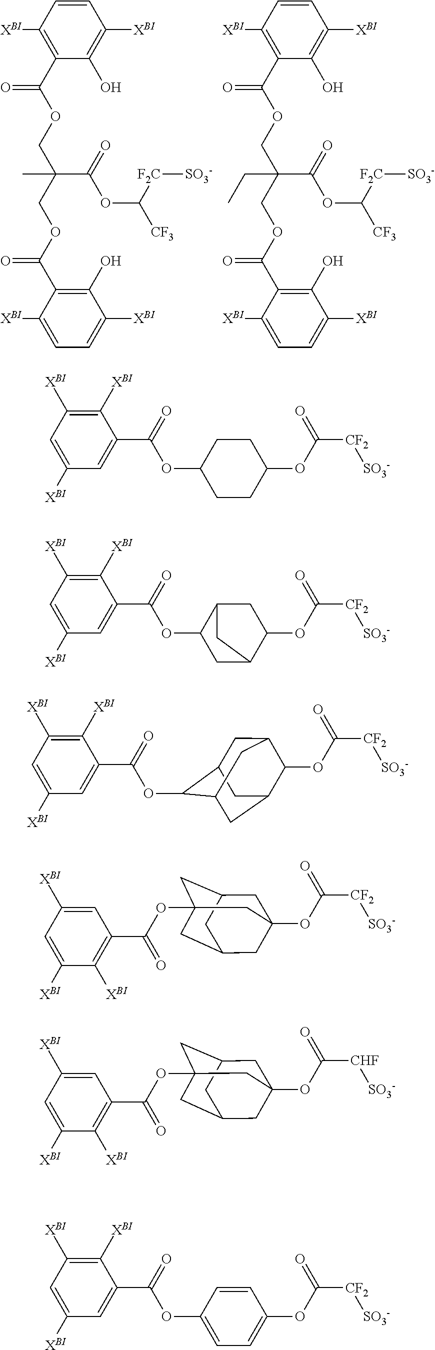

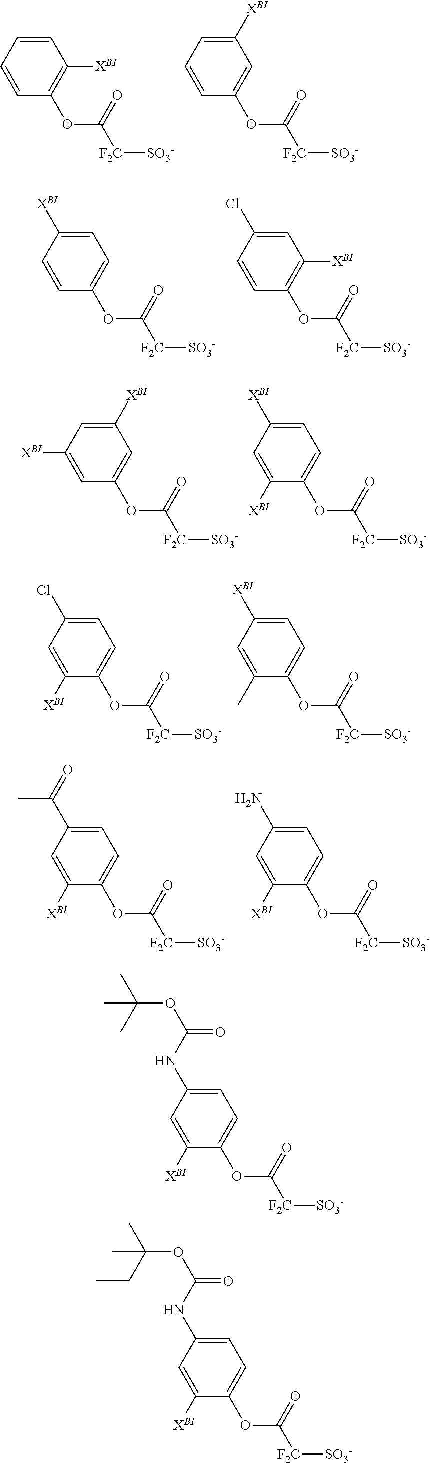

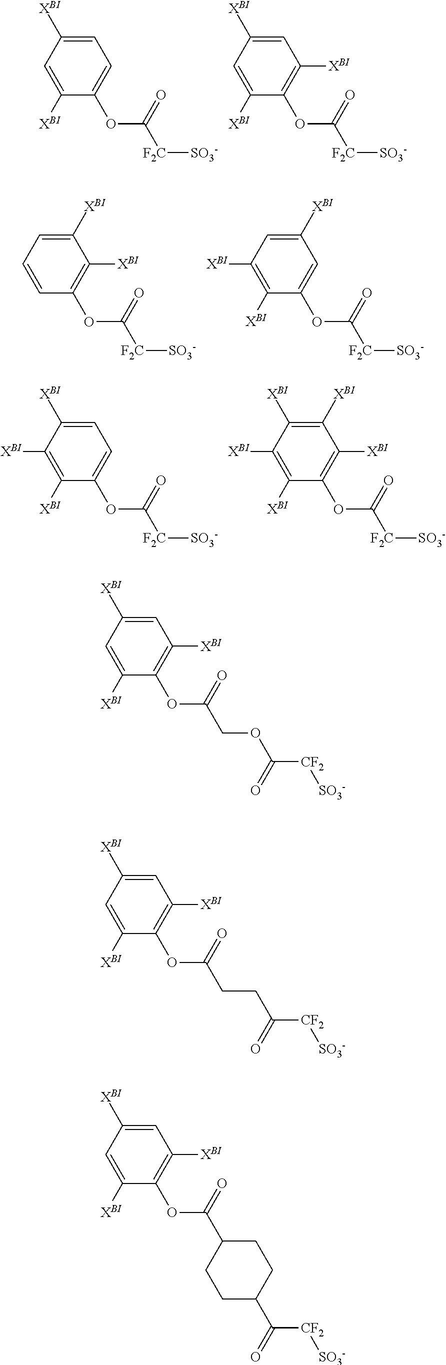

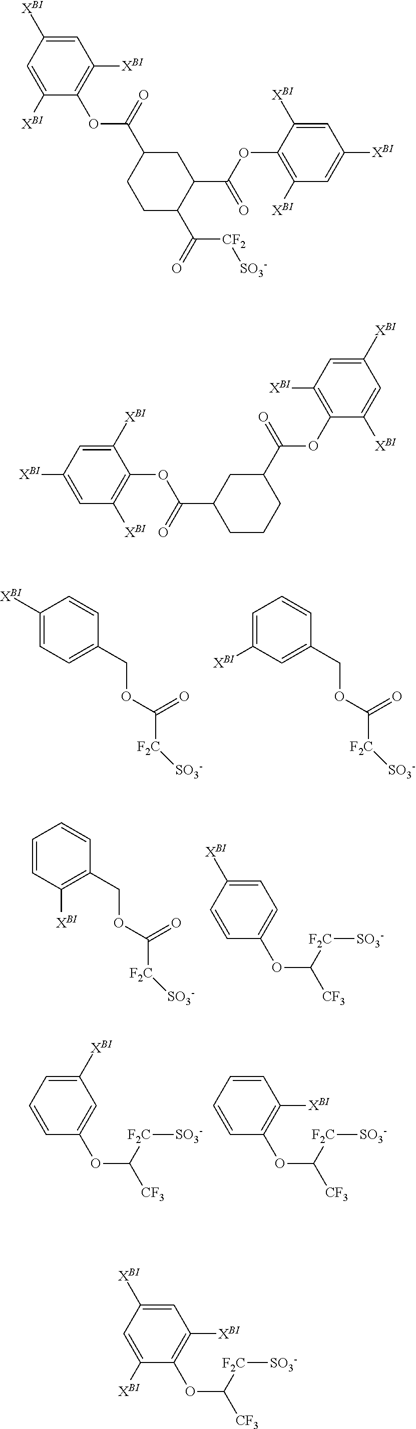

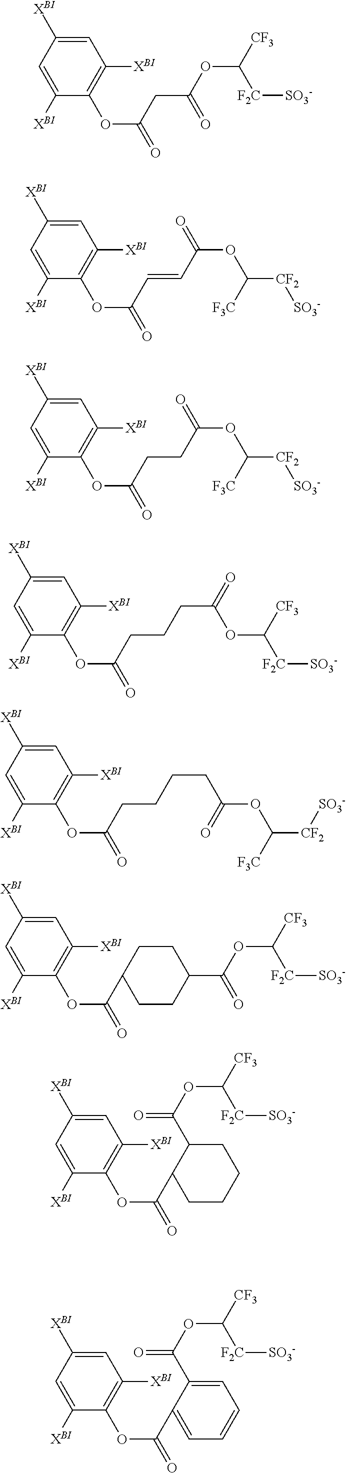

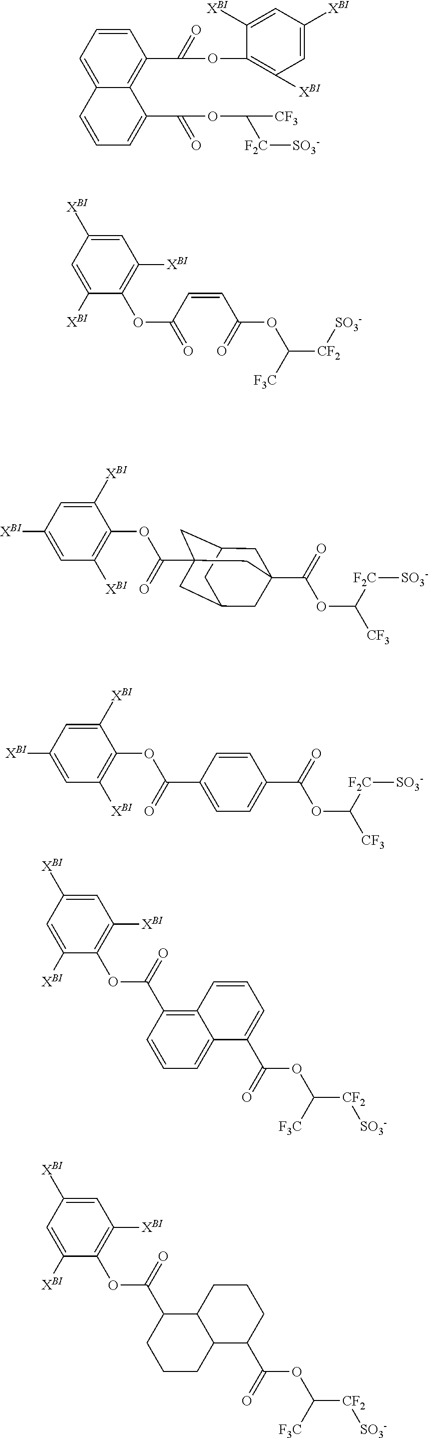

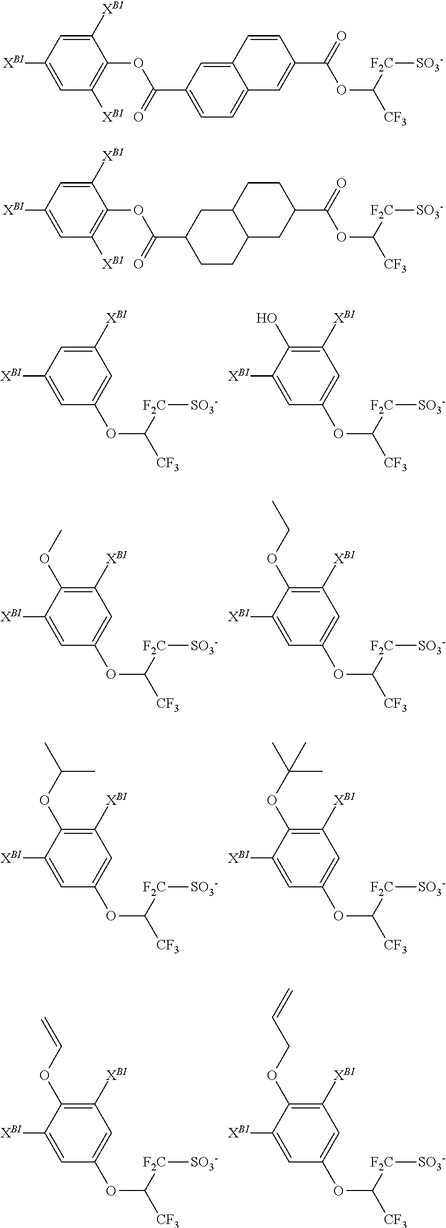

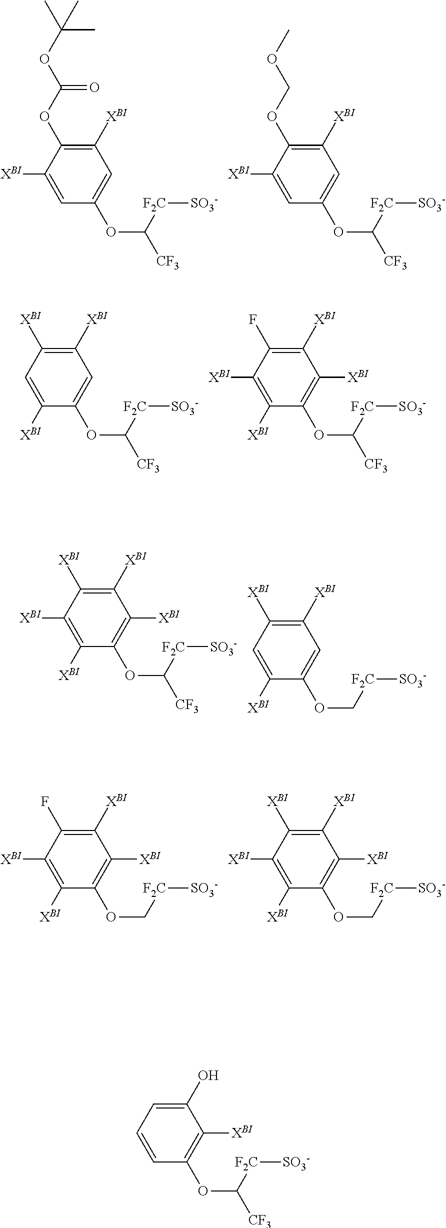

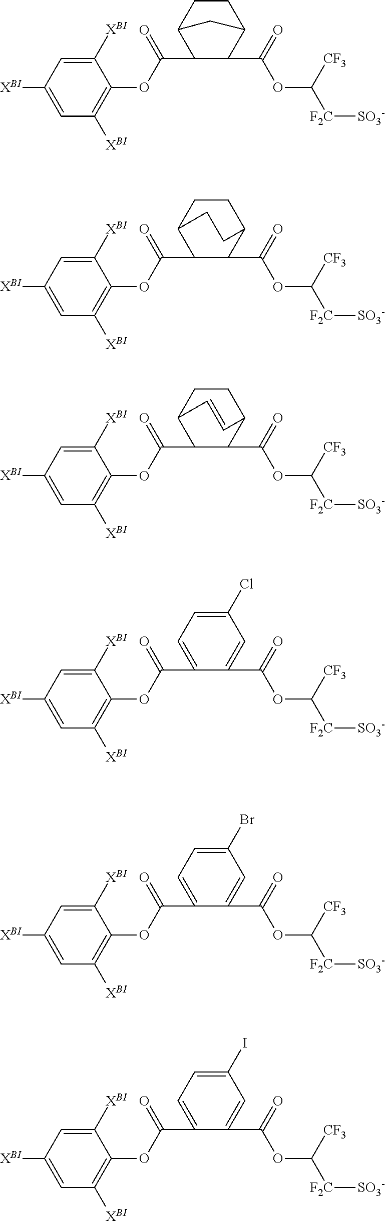

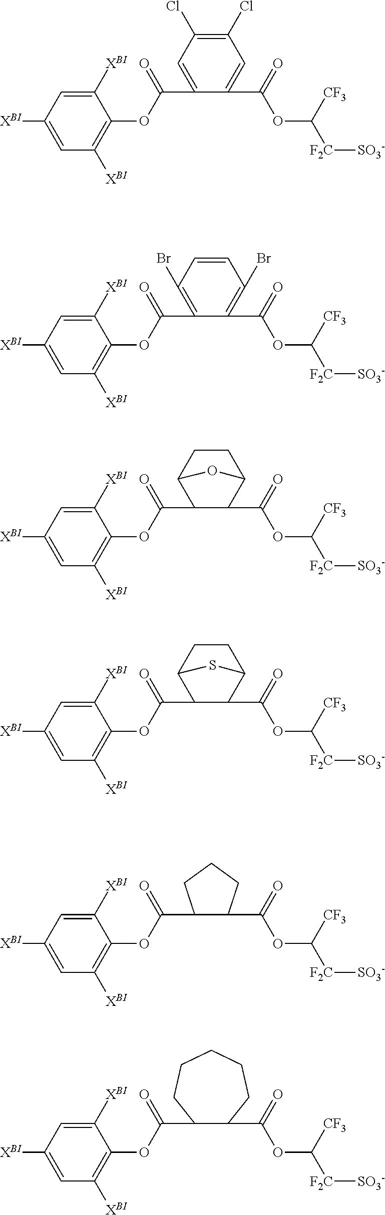

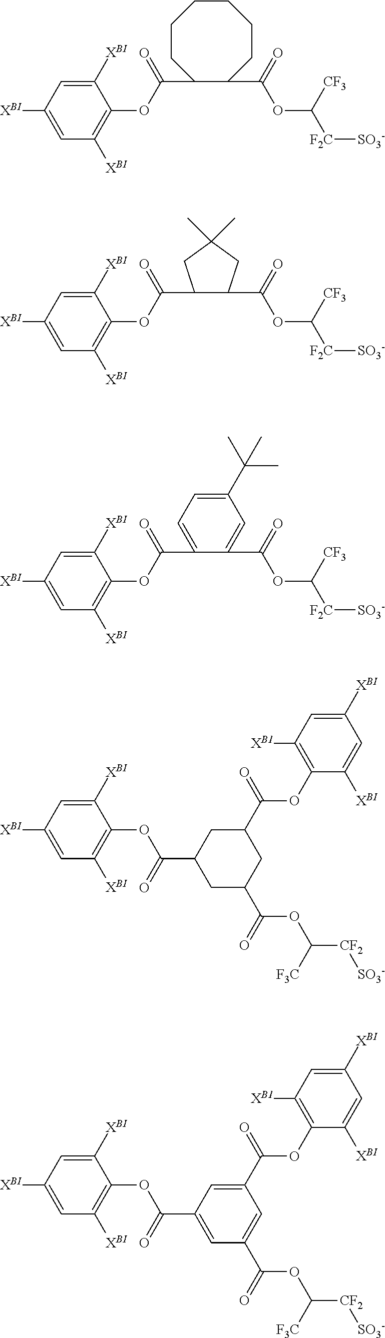

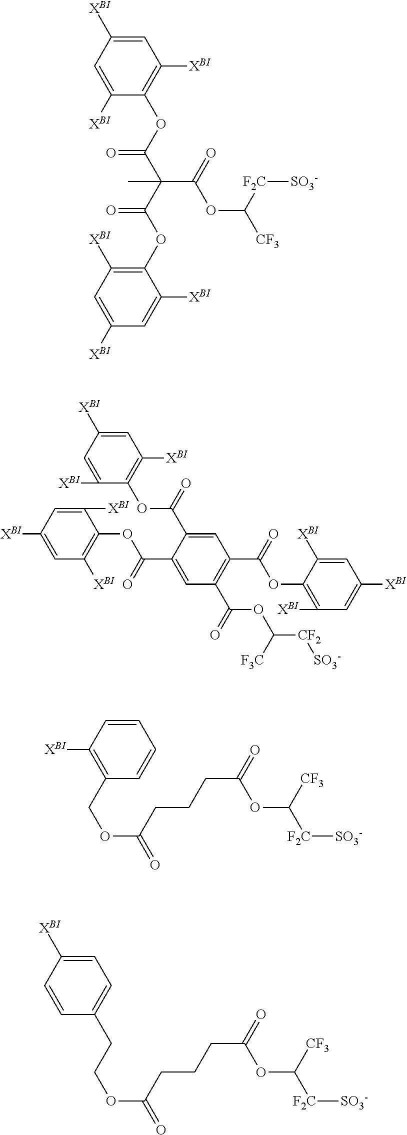

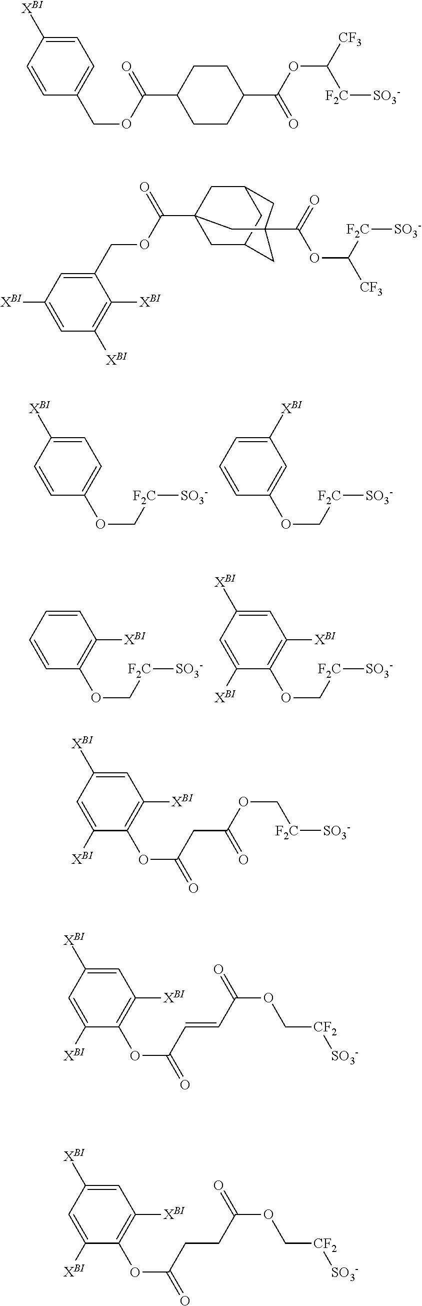

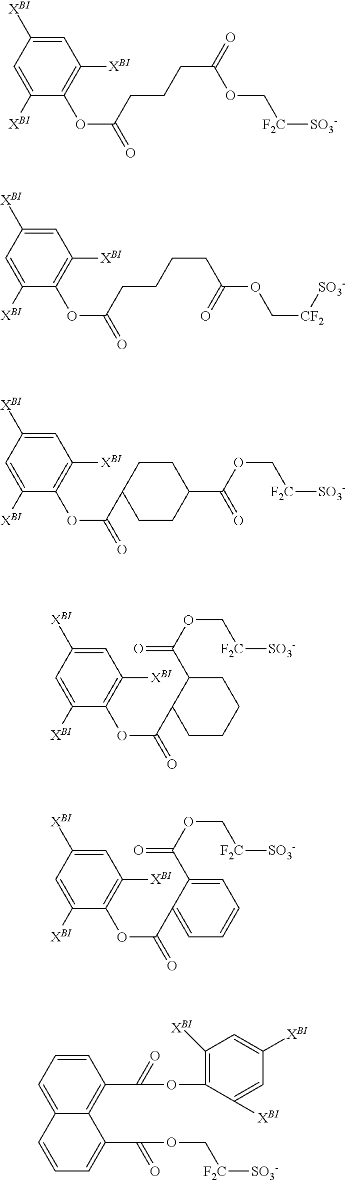

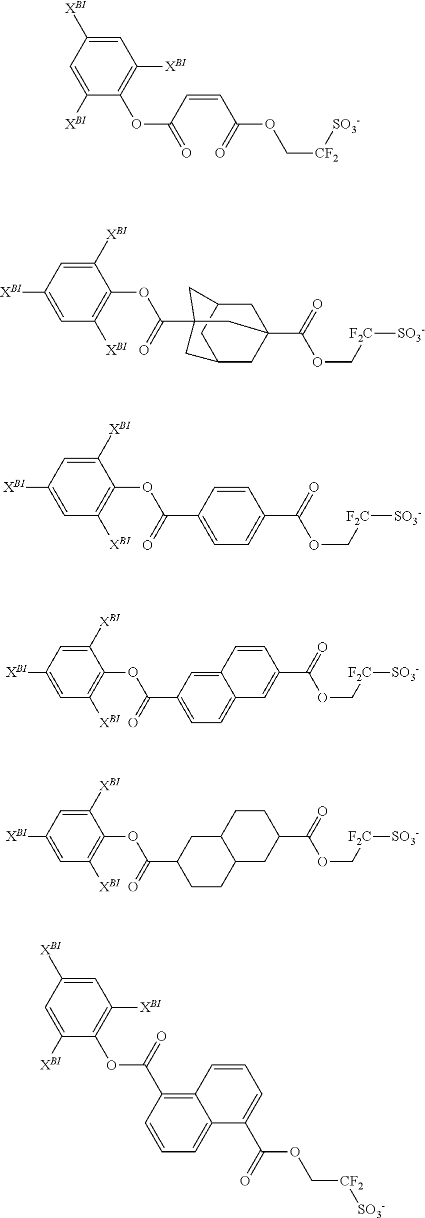

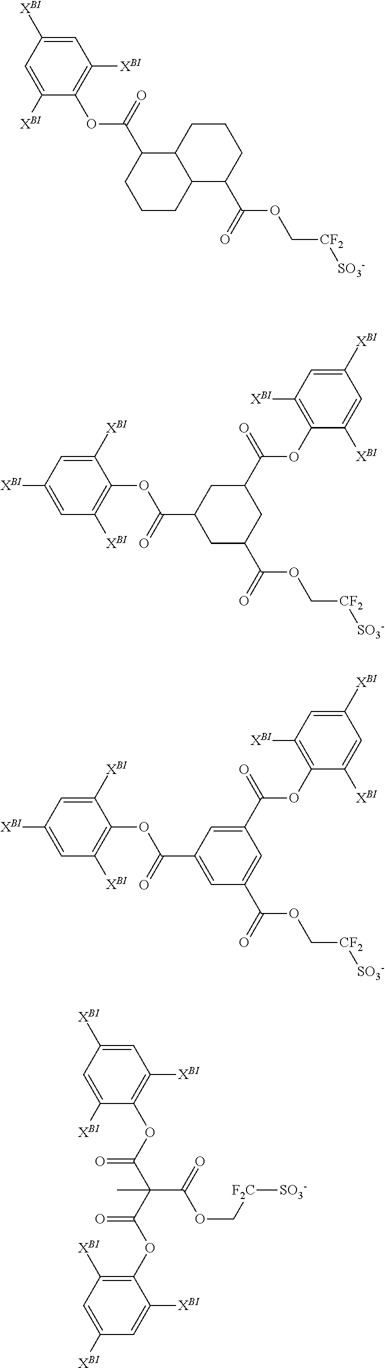

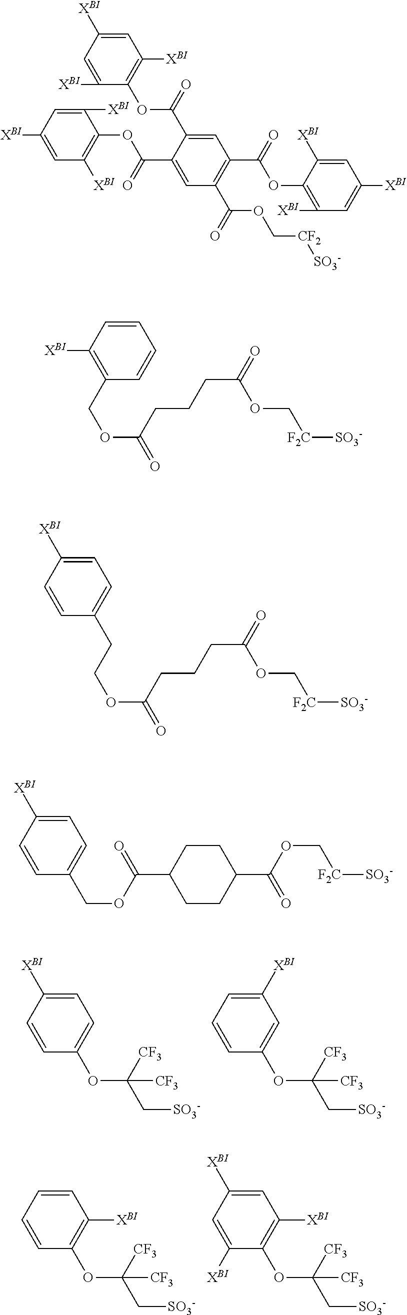

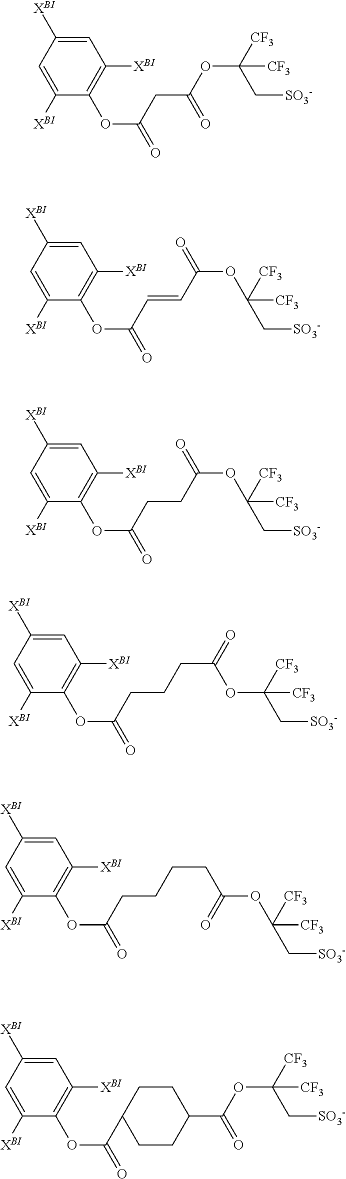

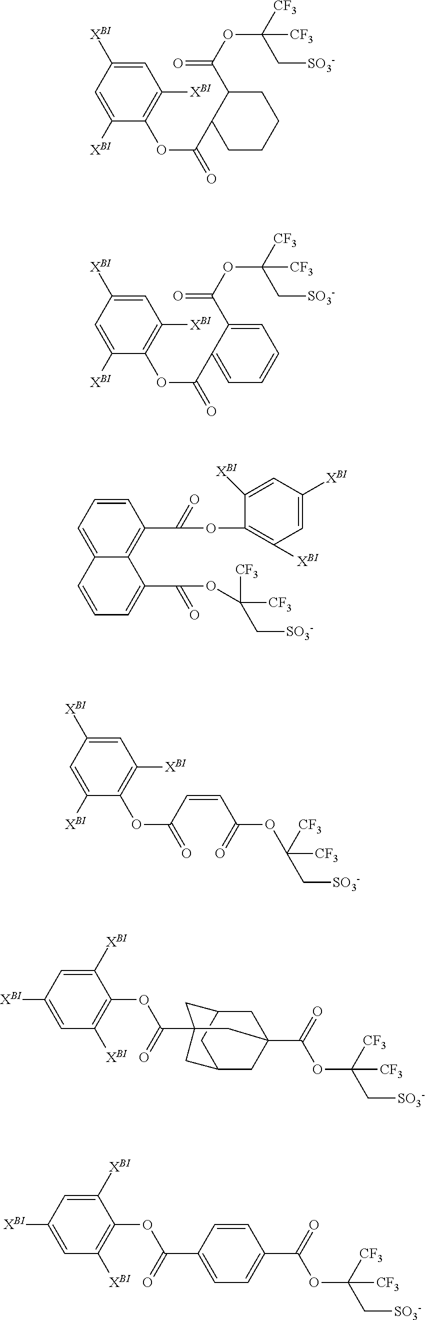

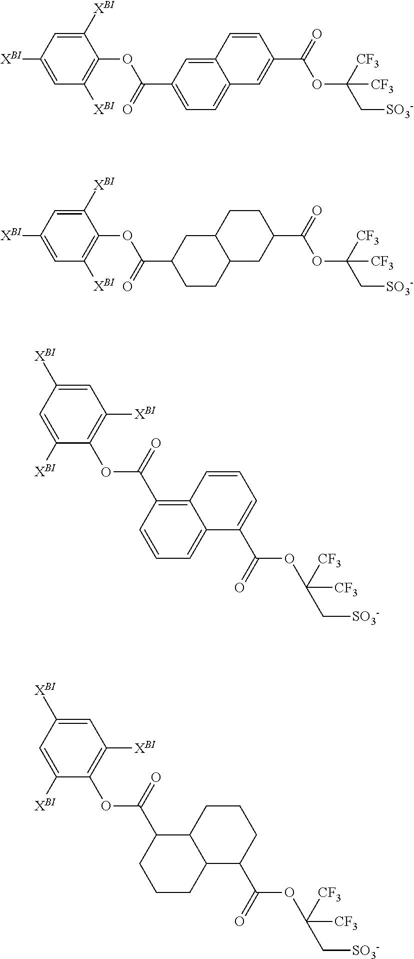

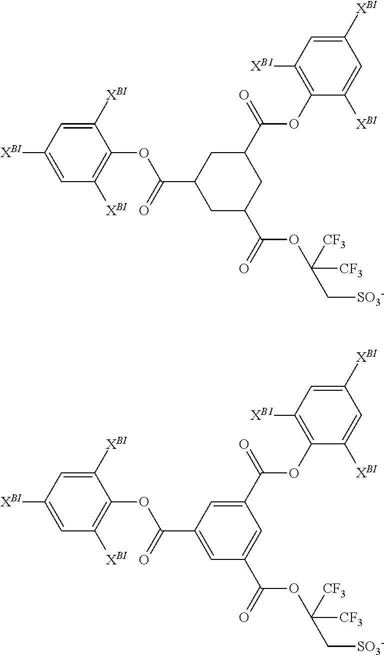

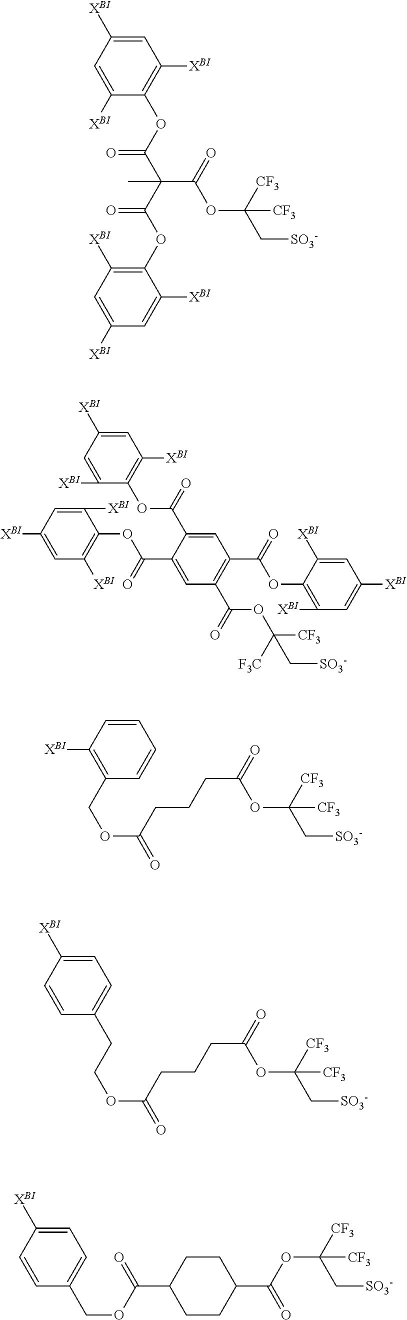

When the inventive resist material contains the additive-type acid generator, the content thereof is preferably 0.1 to 50 parts by mass, and more preferably 1 to 40 parts by mass, relative to 100 parts by mass of the base polymer. The base polymer having any one of the repeating units f1 to f3 and/or the additive-type acid generator enables the inventive resist material to function as the chemically amplified resist material.

Organic Solvent

The inventive resist material may comprise an organic solvent. The organic solvent is not particularly limited as long as the solvent can dissolve each of the aforementioned components and each component described later. Examples of the organic solvent include, as described in paragraphs [0144] to [0145] of JP 2008-111103 A, ketones, such as cyclohexanone, cyclopentanone, methyl-2-n-pentyl ketone, and 2-heptanone; alcohols, such as 3-methoxybutanol, 3-methyl-3-methoxybutanol, 1-methoxy-2-propanol, 1-ethoxy-2-propanol, and diacetone alcohol; ethers, such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters, such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones, such as γ-butyrolactone.

The content of the organic solvent in the inventive resist material is preferably 100 to 10,000 parts by mass, and more preferably 200 to 8,000 parts by mass, relative to 100 parts by mass of the base polymer. The organic solvent may be used singly, or may be used with mixing two or more kinds thereof.

Other Components

The inventive resist material may contain, in addition to the aforementioned components, a surfactant, a dissolution inhibitor, a crosslinker, a quencher other than the sulfonium salt A (hereinafter, referred to as the other quencher), a water repellency enhancer, acetylene alcohols, and the like.

Examples of the surfactant include surfactants described in paragraphs [0165] to [0166] of JP 2008-111103 A. Adding the surfactant can further improve or regulate the coatability of the resist material. When the inventive resist material contains the surfactant, the content thereof is preferably 0.0001 to parts by mass relative to 100 parts by mass of the base polymer. The surfactant may be used singly, or may be used in combination of two or more kind thereof.

When the inventive resist material is the positive type resist material, blending a dissolution inhibitor can further increase the difference in the dissolution rate between the exposed portion and the unexposed portion to further improve the resolution. Examples of the dissolution inhibitor include: a compound having a molecular weight of preferably 100 to 1,000, more preferably 150 to 800, and having two or more phenolic hydroxy groups in the molecule, wherein 0 to 100 mol % of all the hydrogen atoms in the phenolic hydroxy groups are substituted with an acid-labile group; or a compound having a carboxy group in the molecule, wherein 50 to 100 mol % in average of all the hydrogen atoms of the carboxy groups are substituted with an acid-labile group. Specific examples thereof include compounds in which hydrogen atoms of hydroxy groups or carboxy groups in bisphenol A, trisphenol, phenolphthalein, cresol novolac, naphtharenecarboxylic acid, adamantanecarboxylic acid, and cholic acid are substituted with an acid-labile group. These dissolution inhibitors are described in paragraphs [0155] to [0178] of JP 2008-122932 A, for example.

When the inventive resist material is the positive type resist material and contains the dissolution inhibitor, the content thereof is preferably 0 to 50 parts by mass, and more preferably 5 to 40 parts by mass, relative to 100 parts by mass of the base polymer. The dissolution inhibitor may be used singly, or may be used in combination of two or more kind thereof.

Meanwhile, when the inventive resist material is the negative-type resist material, adding a crosslinker can reduce the dissolution rate in the exposed portion to obtain a negative pattern. Examples of the crosslinker include: epoxy compounds, melamine compounds, guanamine compounds, glycoluril compounds, or urea compounds each of which is substituted with at least one group selected from a methylol group, an alkoxymethyl group, and an acyloxymethyl group; isocyanate compounds; azide compounds; or compounds having a double bond, such as an alkenyloxy group. These compounds may be used as an additive, or may be introduced into the polymer side chain as a pendant group. Compounds having a hydroxy group can also be used as the crosslinker.

Examples of the epoxy compound include tris(2,3-epoxypropyl)isocyanurate, trimethylolmethane triglycidyl ether, trimethylolpropane triglycidyl ether, and triethylolethane triglycidyl ether.

Examples of the melamine compound include hexamethylolmelamine, hexamethoxymethylmelamine, a compound in which 1 to 6 methylol groups in hexamethylolmelamine are methoxymethylated or a mixture thereof, hexamethoxymethylmelamine, hexaacyloxymethylmelamine, and a compound in which 1 to 6 methylol groups in hexamethylolmelamine are acyloxymethylated or a mixture thereof.

Examples of the guanamine compound include tetramethylolguanamine, tetramethoxymethylguanamine, a compound in which 1 to 4 methylol groups in tetramethylolguanamine are methoxymethylated or a mixture thereof, tetramethoxyethylguanamine, tetraacyloxyguanamine, and a compound in which 1 to 4 methylol groups in tetramethylolguanamine are acyloxymethylated or a mixture thereof.

Examples of the glycoluril compound include tetramethylolglycoluril, tetramethoxyglycoluril, tetramethoxymethylglycoluril, a compound in which 1 to 4 methylol groups in tetramethylolglycoluril are methoxymethylated or a mixture thereof, and a compound in which 1 to 4 methylol groups in tetramethylolglycoluril are acyloxymethylated or a mixture thereof.

Examples of the urea compound include tetramethylolurea, tetramethoxymethylurea, a compound in which 1 to 4 methylol groups in tetramethylolurea are methoxymethylated or a mixture thereof, and tetramethoxyethylurea.

Examples of the isocyanate compound include tolylene diisocyanate, diphenylmethane diisocyanate, hexamethylene diisocyanate, and cyclohexane diisocyanate.

Examples of the azide compound include 1,1′-biphenyl-4,4′-bisazide, 4,4′-methylidenebisazide, and 4,4′-oxybisazide.

Examples of the compound having an alkenyloxy group include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylolpropane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, and trimethylolpropane trivinyl ether.

When the inventive resist material is the negative-type resist material and contains the crosslinker, the content thereof is preferably 0.1 to 50 parts by mass, and more preferably 1 to 40 parts by mass, relative to 100 parts by mass of the base polymer. The crosslinker may be used singly, or may be used in combination of two or more kind thereof.

Examples of the other quencher include conventional basic compounds. Examples of the conventional basic compound include primary, secondary, or tertiary aliphatic amines, mixed amines, aromatic amines, heterocyclic amines, nitrogen-containing compounds having a carboxy group, nitrogen-containing compounds having a sulfonyl group, nitrogen-containing compounds having a hydroxy group, nitrogen-containing compounds having a hydroxyphenyl group, alcoholic nitrogen-containing compounds, amides, imides, and carbamates. Specifically, preferable are: primary, secondary, or tertiary amine compounds described in paragraphs [0146] to [0164] in JP 2008-111103 A; particularly, amine compounds having a hydroxy group, an ether bond, an ester bond, a lactone ring, a cyano group, or a sulfonate ester bond; or compounds having a carbamate group described in JP 3790649 B. Adding such a basic compound can further reduce the diffusion rate of the acid in the resist film and modify the shape, for example.

Examples of the other quencher also include onium salts such as sulfonium salts, iodonium salts, and ammonium salts of a sulfonic acid and a carboxylic acid having no-fluorinated α-position, described in JP 2008-158339 A. Although the α-position-fluorinated sulfonic acid, imide acid, or methide acid is required for deprotecting the acid-labile group of the carboxylate ester, a sulfonic acid or a carboxylic acid having no-fluorinated α-position are released through salt exchange with the onium salt having no-fluorinated α-position. Since the sulfonic acid and carboxylic acid having no-fluorinated α-position do not cause deprotection reaction, such onium salts function as a quencher.

Examples of the other quencher further include a polymer quencher described in JP 2008-239918 A. This polymer quencher are segregated on a resist film surface to improve rectangularity of the resist pattern. The polymer quencher also has an effect of preventing film reduction of a pattern and rounding of a pattern top when a protective film for immersion exposure is applied.

When the inventive resist material contains the other quencher, the content thereof is preferably 0 to 5 parts by mass, and more preferably 0 to 4 parts by mass, relative to 100 parts by mass of the base polymer. The other quencher may be used singly, or may be used in combination of two or more kind thereof.

The water repellency enhancer improves water repellency on the resist film surface, and can be used for immersion lithography without a top coating. As the water repellency enhancer, a polymer having a fluorinated alkyl group, a polymer with a specific structure having a 1,1,1,3,3,3-hexafluoro-2-propanol residue, etc. are preferable, and water repellency enhancers exemplified in JP 2007-297590 A, JP 2008-111103 A, etc. are more preferable. The water repellency enhancer is necessarily dissolved in an alkaline developer or an organic solvent developer. The aforementioned specific water repellency enhancer having a 1,1,1,3,3,3-hexafluoro-2-propanol residue has good solubility in the developer. A polymer having a repeating unit having an amino group or an amine salt as a water repellency enhancer is highly effective in preventing evaporation of the acid during post exposure bake (PEB) to prevent the developed hole pattern from failure of opening. When the inventive chemically amplified resist material contains the above water repellency enhancer, the content thereof is preferably 0 to parts by mass, and more preferably 0.5 to 10 parts by mass, relative to 100 parts by mass of the base polymer. The water repellency enhancer may be used singly, or may be used in combination of two or more kind thereof.

Examples of the acetylene alcohols include those described in paragraphs [0179] to [0182] in JP 2008-122932 A. When the inventive chemically amplified resist material contains the acetylene alcohols, the content thereof is preferably 0 to 5 parts by mass relative to 100 parts by mass of the base polymer. The acetylene alcohols may be used singly, or may be used in combination of two or more kind thereof.

Patterning Process

When the inventive resist material is used for manufacturing various integrated circuits, known lithographic technology can be applied. Examples of the patterning process include a method comprising steps of: forming a resist film on a substrate by using the aforementioned chemically amplified resist material; exposing the resist film to high-energy ray; and developing the exposed resist film by using a developer.

First, the inventive resist material is applied on a substrate for integrated circuit manufacturing (such as Si, SiO₂, SiN, SiON, TiN, WSi, BPSG, SOG, and an organic anti-reflective film) or a substrate for mask circuit manufacturing (such as Cr, CrO, CrON, MoSi₂, and SiO₂) by an appropriate coating method such as spin-coating, roll-coating, flow-coating, dip-coating, spray-coating, and doctor-coating so that the coating film thickness is 0.01 to 2 μm. This film is pre-baked on a hot plate at preferably 60 to 150° C. for 10 seconds to 30 minutes, more preferably at 80 to 120° C. for 30 seconds to 20 minutes to form a resist film.

Then, the resist film is exposed by using high-energy ray. Examples of the high-energy ray include ultraviolet ray, far ultraviolet ray, electron beam (EB), extreme ultraviolet ray (EUV) having a wavelength of 3 to 15 nm, X-ray, soft X-ray, excimer laser light, γ-ray, and synchrotron radiation. When ultraviolet ray, far ultraviolet ray, EUV, X-ray, soft X-ray, excimer laser light, γ-ray, synchrotron radiation, etc. is used as the high-energy ray, irradiation is performed directly or using a mask for forming a target pattern so that the exposure dose is preferably approximately 1 to 200 mJ/cm², more preferably approximately 10 to 100 mJ/cm². When EB is used as the high-energy ray, writing is performed directly or by using a mask for forming a target pattern at an exposure dose of preferably approximately 0.1 to 300 μC/cm², more preferably approximately 0.5 to 200 μC/cm². The inventive resist material is particularly suitable for fine pattering with, among the high-energy rays, KrF excimer laser light, ArF excimer laser light, EB, EUV, X-ray, soft X-ray, γ-ray, and synchrotron radiation. Among them, KrF excimer laser light, ArF excimer laser light, EB, or EUV having a wavelength of 3 to 15 nm is preferably used, and the inventive resist material is particularly suitable for fine patterning with EB or EUV.

After the exposure, PEB may be performed on a hot plate or in an oven at preferably 30 to 150° C. for 10 seconds to 30 minutes, more preferably 50 to 120° C. for 30 seconds to 20 minutes. The PEB may not be performed.

After the exposure or the PEB, the exposed resist film is developed by a common method such as a dip method, a puddle method, and a spray method for 3 seconds to 3 minutes, preferably 5 seconds to 2 minutes to form a target pattern. For the development, used are a developer of a 0.1 to 10 mass %, preferably 2 to 5 mass %, alkaline aqueous solution such as tetramethylammonium hydroxide (TMAH), tetraethylammonium hydroxide, tetrapropylammonium hydroxide, and tetrabutylammonium hydroxide. In a case of the positive-type resist material, the light-irradiated portion is dissolved in the developer and the unexposed portion is not dissolved to form a target positive-type pattern on the substrate. In a case of the negative-type resist material, in contrast to the case of the positive-type resist material, the light-irradiated portion is insoluble in the developer and the unexposed portion is dissolved.

A negative pattern can also be obtained by using the positive-type resist material containing the base polymer having an acid-labile group with the organic solvent development. Examples of the developer used in this case include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. These organic solvents can be used singly, or used with mixing two or more kinds thereof.

After the development, rinsing is performed. The rinsing liquid is preferably a solvent that mixes with the developer and does not dissolve the resist film. Preferably used as such a solvent are alcohols having 3 to 10 carbon atoms, ether compounds having 8 to 12 carbon atoms, alkanes, alkenes, or alkynes having 6 to 12 carbon atoms, and aromatic solvents.

Examples of the alcohols having 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol.

Examples of the ether compounds having 8 to 12 carbon atoms include di-n-butyl ether, diisobutyl ether, di-sec-butyl ether, di-n-pentyl ether, diisopentyl ether, di-sec-pentyl ether, di-tert-pentyl ether, and di-n-hexyl ether.

Examples of the alkanes having 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Examples of the alkenes having 6 to 12 carbon atoms include hexene, heptene, octene, cyclohexene, methylcyclohexene, dimethylcyclohexene, cycloheptene, and cyclooctene. Examples of the alkynes having 6 to 12 carbon atoms include hexyne, heptyne, and octyne.

Examples of the aromatic solvents include toluene, xylene, ethylbenzene, isopropylbenzene, tert-butylbenzene, and mesitylene.

The rinsing can reduce collapse of the resist pattern and occurrence of defects. Rinsing is not essential, and no rinsing can reduce the use amount of the solvent.

The hole pattern or trench pattern after the development can be shrunk by thermal flow, RELACS technology, or DSA technology. A shrinking agent is applied on the hole pattern, and during baking, an acid catalyst is diffused from the resist film to cause crosslinking of the shrinking agent on the resist film surface, and the shrinking agent adheres to the side wall of the hole pattern. The baking temperature is preferably 70 to 180° C., and more preferably 80 to 170° C. The baking time is preferably 10 to 300 seconds. The extra shrinking agent is removed to shrink the hole pattern.

### EXAMPLE

Hereinafter, the present invention will be specifically described by showing Synthesis Examples, Examples, and Comparative Examples, but the present invention is not limited to the following Examples.

The structures of quenchers Q-1 to Q-28 used for resist materials are shown below.

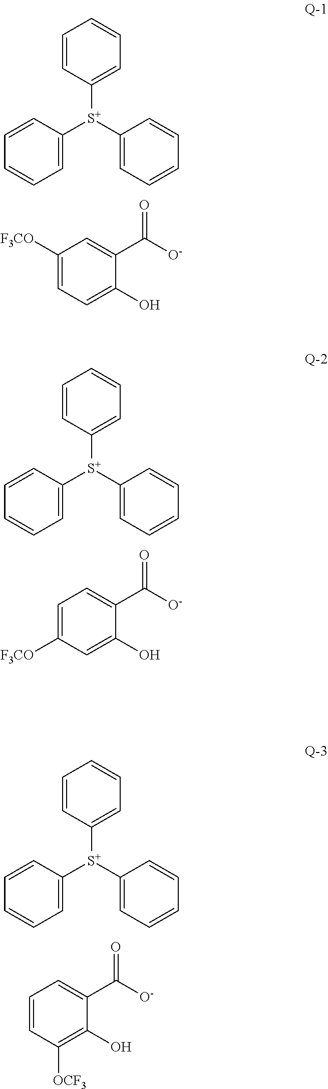 [SMILES: c1ccc([S+](c2ccccc2)c2ccccc2)cc1 ; O=C([O-])c1cc(OC(F)(F)F)ccc1O ; c1ccc([S+](c2ccccc2)c2ccccc2)cc1 ; O=C([O-])c1ccc(OC(F)(F)F)cc1O ; c1ccc([S+](c2ccccc2)c2ccccc2)cc1 ; O=C([O-])c1cccc(OC(F)(F)F)c1O]

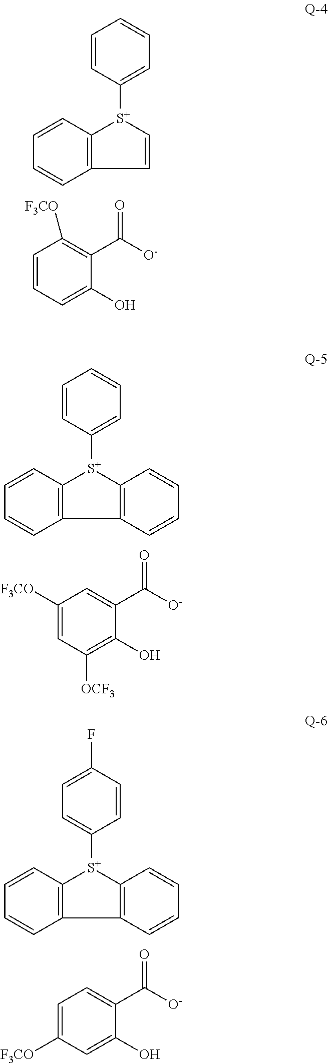 [SMILES: c1ccc(-[s+]2ccc3ccccc32)cc1 ; O=C([O-])c1c(O)cccc1OC(F)(F)F ; c1ccc(-[s+]2c3ccccc3c3ccccc32)cc1 ; O=C([O-])c1cc(OC(F)(F)F)cc(OC(F)(F)F)c1O ; Fc1ccc(-[s+]2c3ccccc3c3ccccc32)cc1 ; O=C([O-])c1ccc(OC(F)(F)F)cc1O]

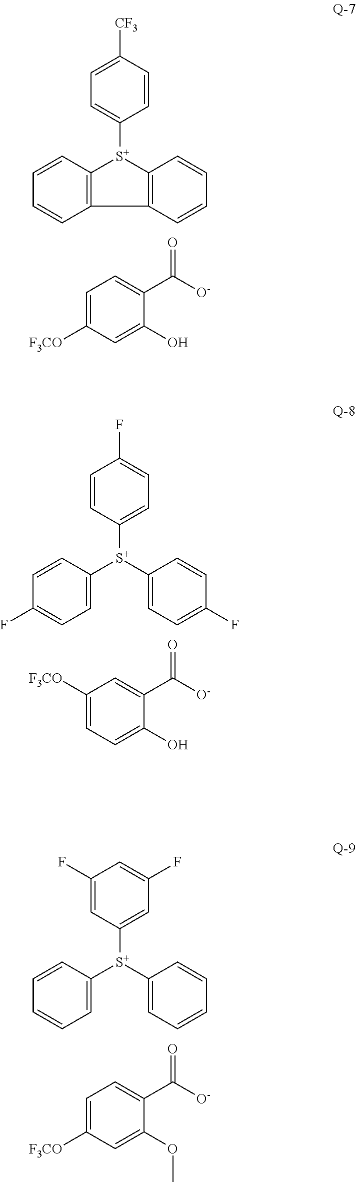 [SMILES: FC(F)(F)c1ccc(-[s+]2c3ccccc3c3ccccc32)cc1 ; O=C([O-])c1ccc(OC(F)(F)F)cc1O ; Fc1ccc([S+](c2ccc(F)cc2)c2ccc(F)cc2)cc1 ; O=C([O-])c1cc(OC(F)(F)F)ccc1O ; Fc1cc(F)cc([S+](c2ccccc2)c2ccccc2)c1 ; COc1cc(OC(F)(F)F)ccc1C(=O)[O-]]

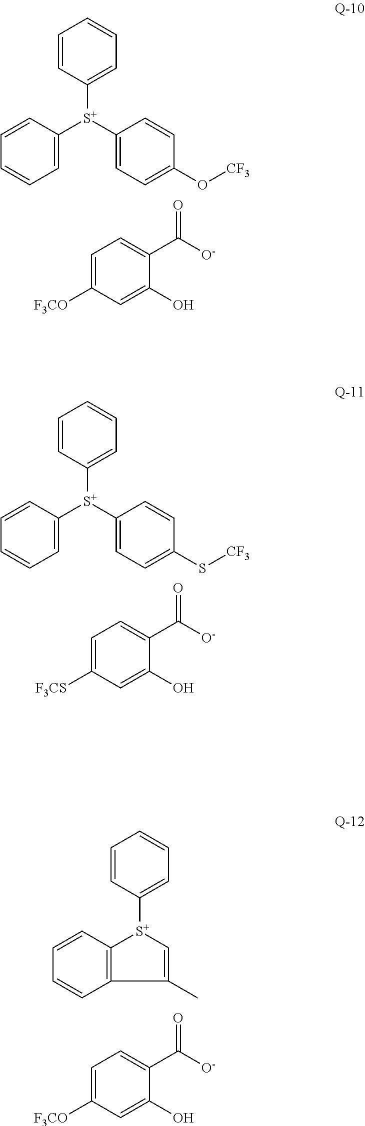

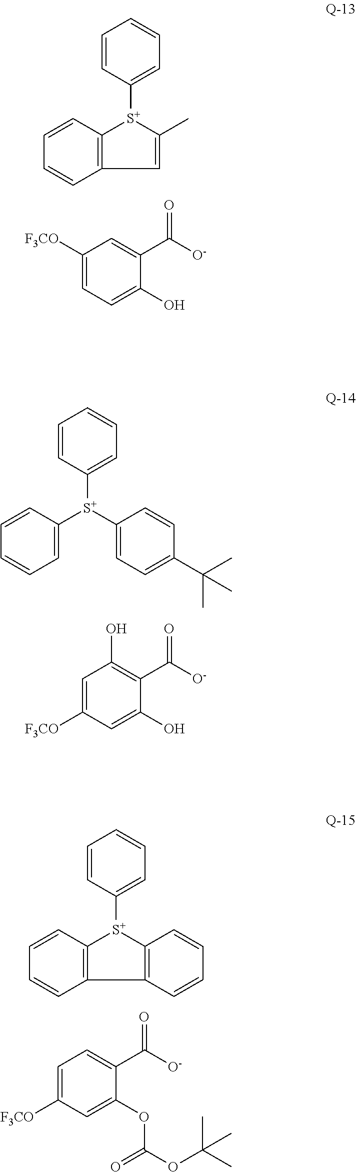 [SMILES: Cc1cc2ccccc2[s+]1-c1ccccc1 ; O=C([O-])c1cc(OC(F)(F)F)ccc1O ; CC(C)(C)c1ccc([S+](c2ccccc2)c2ccccc2)cc1 ; O=C([O-])c1c(O)cc(OC(F)(F)F)cc1O ; c1ccc(-[s+]2c3ccccc3c3ccccc32)cc1 ; CC(C)(C)OC(=O)Oc1cc(OC(F)(F)F)ccc1C(=O)[O-]]

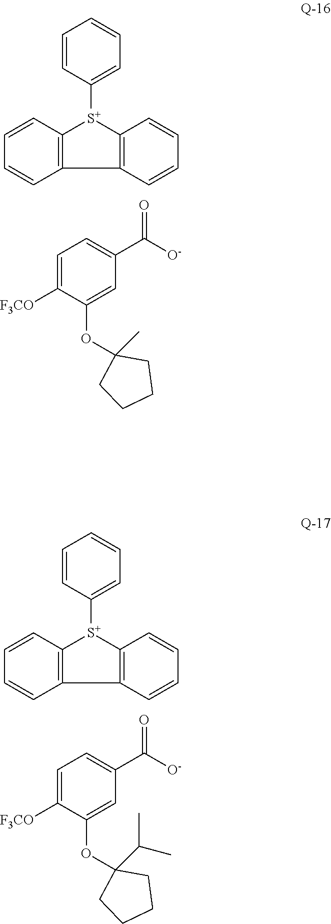 [SMILES: c1ccc(-[s+]2c3ccccc3c3ccccc32)cc1 ; CC1(Oc2cc(C(=O)[O-])ccc2OC(F)(F)F)CCCC1 ; c1ccc(-[s+]2c3ccccc3c3ccccc32)cc1 ; CC(C)C1(Oc2cc(C(=O)[O-])ccc2OC(F)(F)F)CCCC1]

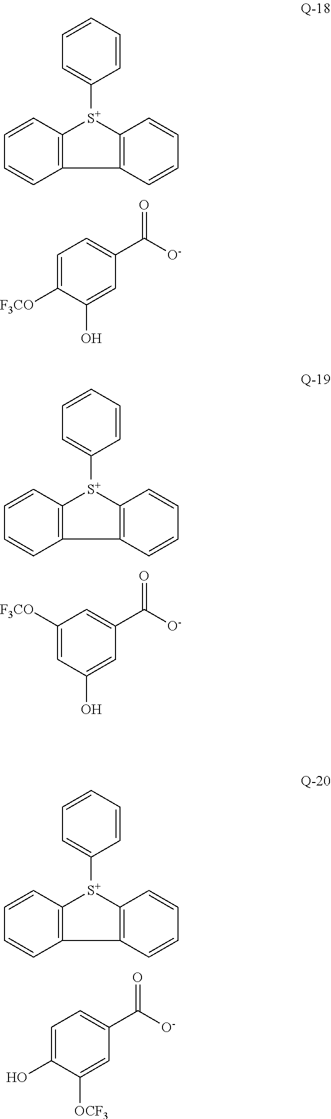 [SMILES: c1ccc(-[s+]2c3ccccc3c3ccccc32)cc1 ; O=C([O-])c1ccc(OC(F)(F)F)c(O)c1 ; c1ccc(-[s+]2c3ccccc3c3ccccc32)cc1 ; O=C([O-])c1cc(O)cc(OC(F)(F)F)c1 ; c1ccc(-[s+]2c3ccccc3c3ccccc32)cc1 ; O=C([O-])c1ccc(O)c(OC(F)(F)F)c1]

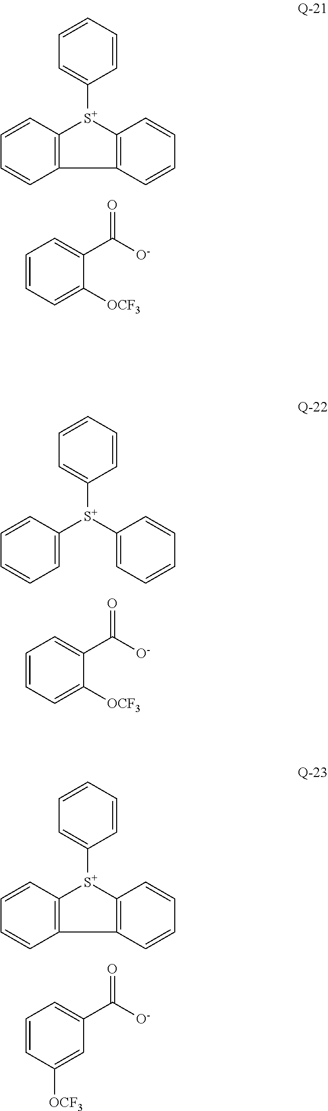 [SMILES: c1ccc(-[s+]2c3ccccc3c3ccccc32)cc1 ; O=C([O-])c1ccccc1OC(F)(F)F ; c1ccc([S+](c2ccccc2)c2ccccc2)cc1 ; O=C([O-])c1ccccc1OC(F)(F)F ; c1ccc(-[s+]2c3ccccc3c3ccccc32)cc1 ; O=C([O-])c1cccc(OC(F)(F)F)c1]

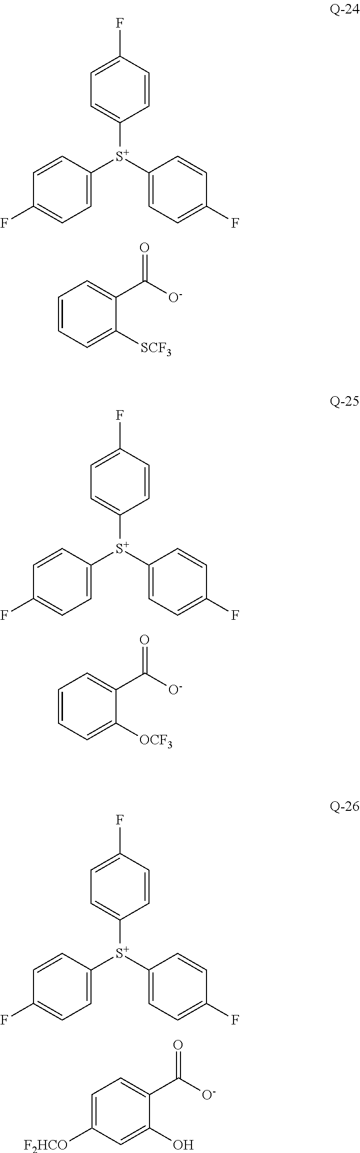

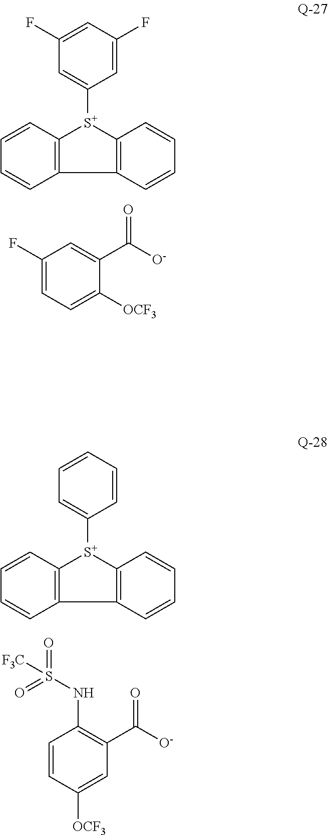 [SMILES: Fc1cc(F)cc(-[s+]2c3ccccc3c3ccccc32)c1 ; O=C([O-])c1cc(F)ccc1OC(F)(F)F ; c1ccc(-[s+]2c3ccccc3c3ccccc32)cc1 ; O=C([O-])c1cc(OC(F)(F)F)ccc1NS(=O)(=O)C(F)(F)F]

### Synthesis Example: Syntheses of Base Polymers (Polymers P-1 to 5)

Each of the monomers was combined to perform a copolymerization reaction in a THF solvent, the product was precipitated with methanol, further washed repeatedly with hexane, then isolated, and dried to obtain each of the base polymers (polymers P-1 to 5) having the following composition. The composition of the obtained base polymer was determined by ¹H-NMR, and Mw and Mw/Mn thereof were determined by GPC (solvent: THF, standard: polystyrene).

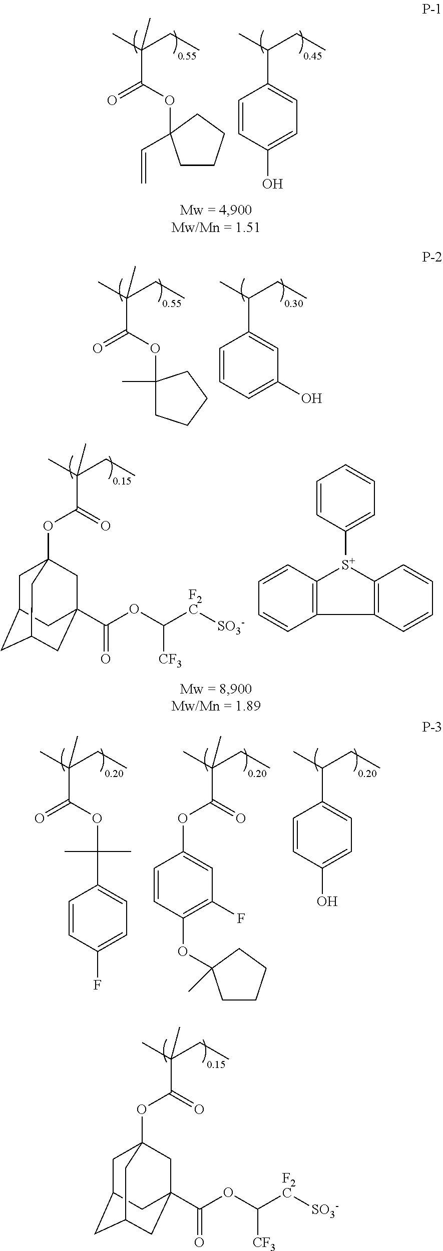

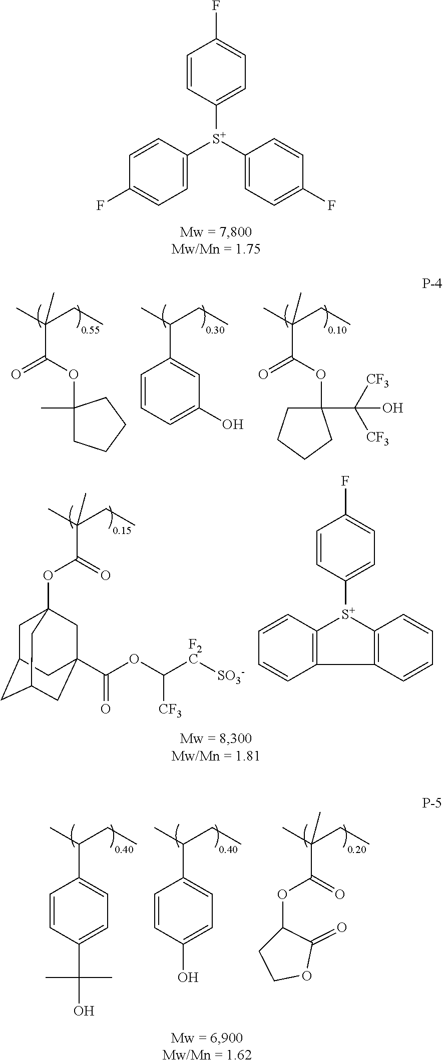

### Examples 1 to 32 and Comparative Examples 1 to 4: Preparation of Resist Material and Its Evaluation

(1) Preparation of Resist Material

A solution dissolving each component at a composition shown in Table 1 and Table 2 was filtered with a filter with 0.2 μm in in size to prepare a resist material. Resist materials of Examples 1 to 20 and 22 to 32 and Comparative Examples 1 to 3 were positive-type resist materials, and resist compositions of Example 21 and Comparative Example 4 were negative-type resist materials.

Each component in Table 1 and Table 2 was as follows.

Organic Solvent:
PGMEA (propylene glycol monomethyl ether acetate)
DAA (diacetone alcohol)
EL (ethyl lactate)

Photoacid generator: PAG-1 to PAG-5

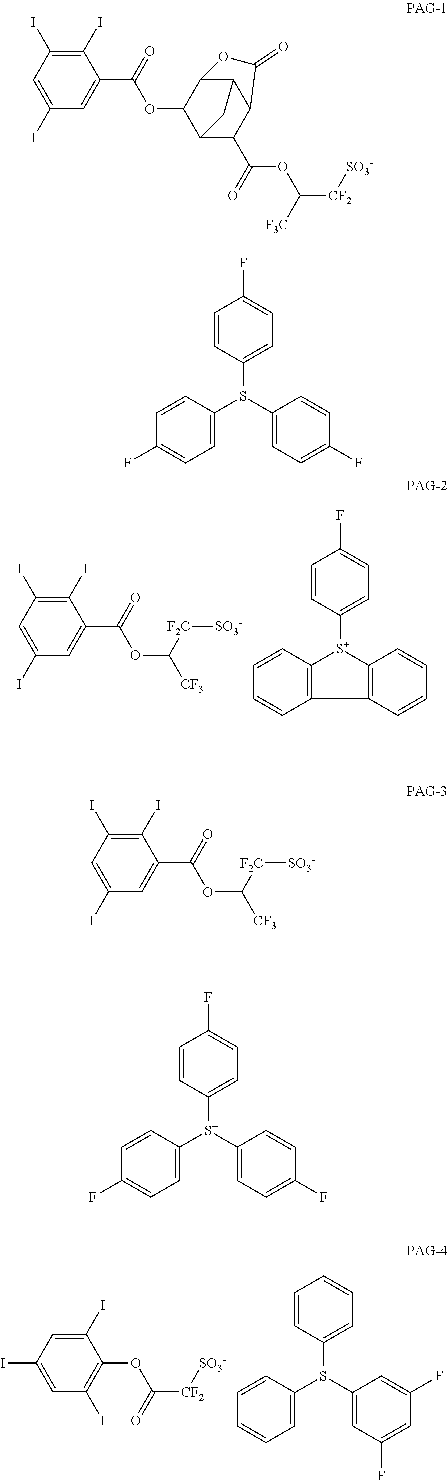

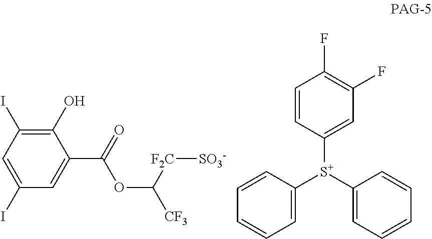

Blended quencher: bQ-1 and bQ-2

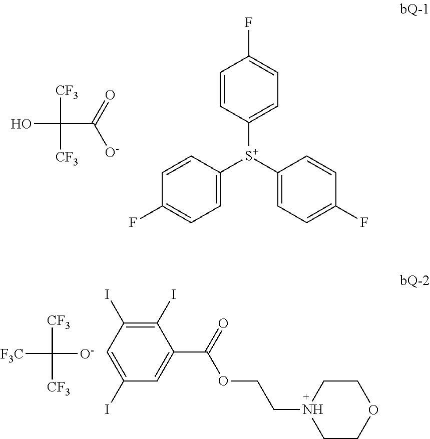 [SMILES: O=C([O-])C(O)(C(F)(F)F)C(F)(F)F ; Fc1ccc([S+](c2ccc(F)cc2)c2ccc(F)cc2)cc1 ; [O-]C(C(F)(F)F)(C(F)(F)F)C(F)(F)F ; O=C(OCCN1CCOCC1)c1cc(I)cc(I)c1I]

Comparative Quencher: cQ-1, cQ-2, and cQ-3

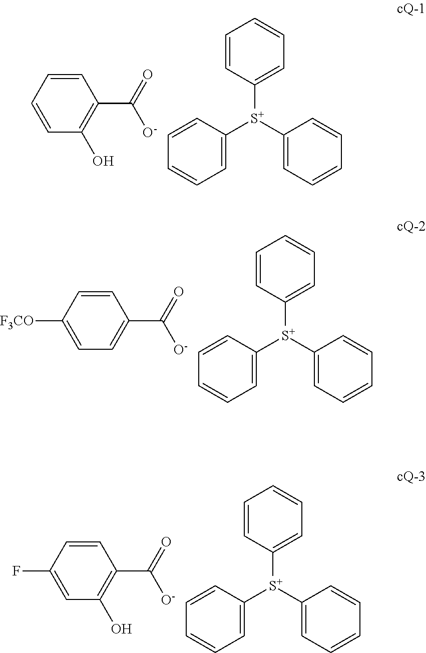 [SMILES: O=C([O-])c1ccccc1O ; c1ccc([S+](c2ccccc2)c2ccccc2)cc1 ; O=C([O-])c1ccc(OC(F)(F)F)cc1 ; c1ccc([S+](c2ccccc2)c2ccccc2)cc1 ; O=C([O-])c1ccc(F)cc1O ; c1ccc([S+](c2ccccc2)c2ccccc2)cc1]

(2) EUV Lithography Evaluation

Each of the resist materials shown in Table 1 and Table 2 was applied by spin-coating on a Si substrate on which a silicon-containing spin-on hard mask SHB-A940 (silicon content of 43 mass %), manufactured by Shin-Etsu Chemical Co., Ltd., was formed with 20 nm in film thickness. The applied resist material was prebaked at 100° C. for 60 seconds using a hot plate to produce a resist film with 60 nm in film thickness. Then, the resist film was exposed by using an EUV scanner NXE3400 (NA 0.33, σ0.9/0.6, quadrupole illumination, hole pattern mask with 44 nm in pitch as on-wafer size and +20% bias), manufactured by ASML Holding N.V. Then, PEB was performed at a temperature shown in Table 1 and Table 2 for 60 seconds on a hot plate, and development was performed with a 2.38 mass % aqueous TMAH solution for 30 seconds to obtain a hole pattern with 22 nm in size in Examples 1 to 20 and 22 to 32 and Comparative Examples 1 to 3, and a dot pattern with 22 nm in size in Example 21 and Comparative Example 4.

Using a length-measurement SEM (CG6300), manufactured by Hitachi High-Technologies Corporation, an exposure dose when the hole or dot pattern was formed with 22 nm in size was measured to specify this exposure dose as a sensitivity. Sizes of 50 holes or dots in this time were measured, and the tripled value (36) of the standard variation (a) calculated from the results was determined as CDU. Table 1 and Table 2 show the results.

TABLE 1

| | | | | | PEB | | |
|---|---|---|---|---|---|---|---|
| | Polymer | Acid generator | Quencher | Organic solvent | temperature | Sensitivity | CDU |
| | (parts by mass) | (parts by mass) | (parts bymass) | (parts by mass) | (° C.) | (mJ/cm²) | (nm) |
| Ex. 1 | P-1 (100) | PAG-1 (30.2) | Q-1 (4.84) | PGMEA (3,000) | 80 | 33 | 3.1 |
| | | | | DAA (500) | | | |
| Ex. 2 | P-1 (100) | PAG-2 (24.8) | Q-2 (4.84) | PGMEA (3,000) | 80 | 34 | 3.1 |
| | | | | DAA (500) | | | |
| Ex. 3 | P-1 (100) | PAG-2 (24.8) | Q-3 (4.84) | PGMEA (3,000) | 80 | 32 | 3.4 |
| | | | | DAA (500) | | | |
| Ex. 4 | P-1 (100) | PAG-2 (24.8) | Q-4 (2.16) | PGMEA (3,000) | 80 | 29 | 3.2 |
| | | | bQ-1 (2.64) | DAA (500) | | | |
| Ex. 5 | P-1 (100) | PAG-2 (24.8) | Q-5 (5.66) | PGMEA (3,000) | 80 | 33 | 3.1 |
| | | | | DAA (500) | | | |
| Ex. 6 | P-1 (100) | PAG-2 (24.8) | Q-6 (5.00) | PGMEA (3,000) | 80 | 31 | 3.3 |
| | | | | DAA (500) | | | |
| Ex. 7 | P-1 (100) | PAG-2 (24.8) | Q-7 (5.40) | PGMEA (3,000) | 80 | 28 | 3.2 |
| | | | | DAA (500) | | | |
| Ex. 8 | P-1 (100) | PAG-2 (24.8) | Q-8 (5.38) | PGMEA (3,000) | 80 | 27 | 3.5 |
| | | | | DAA (500) | | | |
| Ex. 9 | P-1 (100) | PAG-3 (25.7) | Q-9 (2.67) | PGMEA (3,000) | 80 | 29 | 3.5 |
| | | | bQ-2 (4.24) | DAA (500) | | | |
| Ex. 10 | P-1 (100) | PAG-3 (25.7) | Q-10 (5.68) | PGMEA (3,000) | 80 | 28 | 3.2 |
| | | | | DAA (500) | | | |
| Ex. 11 | P-1 (100) | PAG-3 (25.7) | Q-11 (6.00) | PGMEA (3,000) | 80 | 29 | 3.3 |
| | | | | DAA (500) | | | |
| Ex. 12 | P-1 (100) | PAG-3 (25.7) | Q-12 (4.46) | EL (3,000) | 80 | 30 | 3.2 |
| | | | | DAA (500) | | | |
| Ex. 13 | P-1 (100) | PAG-3 (25.7) | Q-13 (4.46) | EL (3,500) | 80 | 32 | 3.2 |
| Ex. 14 | P-1 (100) | PAG-3 (25.7) | Q-14 (5.56) | PGMEA (3,000) | 80 | 32 | 3.3 |
| | | | | DAA (500) | | | |
| Ex. 15 | P-1 (100) | PAG-3 (25.7) | Q-15 (5.82) | PGMEA (3,000) | 80 | 33 | 3.1 |
| | | | | DAA (500) | | | |
| Ex. 16 | P-1 (100) | PAG-3 (25.7) | Q-16 (5.64) | PGMEA (3,000) | 80 | 32 | 3.0 |
| | | | | DAA (500) | | | |
| Ex. 17 | P-1 (100) | PAG-4 (23.2) | Q-17 (5.92) | PGMEA (3,000) | 80 | 33 | 3.0 |
| | | | | DAA (500) | | | |
| Ex. 18 | P-2 (100) | — | Q-8 (5.38) | PGMEA (3,000) | 80 | 34 | 3.1 |
| | | | | DAA (500) | | | |
| Ex. 19 | P-3 (100) | — | Q-8 (5.38) | PGMEA (3,000) | 80 | 33 | 3.0 |
| | | | | DAA (500) | | | |
| Ex. 20 | P-4 (100) | — | Q-8 (5.38) | PGMEA (3,000) | 80 | 32 | 3.1 |
| | | | | DAA (500) | | | |
| Ex. 21 | P-5 (100) | PAG-5 (20) | Q-8 (5.38) | PGMEA (3,000) | 110 | 38 | 4.0 |
| | | | | DAA (500) | | | |
| Comp. | P-1 (100) | PAG-2 (24.8) | cQ-1 (4.00) | PGMEA (3,000) | 80 | 43 | 4.2 |
| Ex. 1 | | | | DAA (500) | | | |
| Comp. | P-1 (100) | PAG-2 (24.8) | cQ-2 (4.68) | PGMEA (3,000) | 80 | 38 | 3.8 |
| Ex. 2 | | | | DAA (500) | | | |
| Comp. | P-1 (100) | PAG-2 (24.8) | cQ-3 (4.18) | PGMEA (3,000) | 80 | 37 | 3.7 |
| Ex. 3 | | | | DAA (500) | | | |
| Comp. | P-5 (100) | PAG-5 (20) | cQ-1 (4.00) | PGMEA (3,000) | 110 | 46 | 5.0 |
| Ex. 4 | | | | DAA (500) | | | |

TABLE 2

| | | | | | PEB | | |
|---|---|---|---|---|---|---|---|
| | Polymer | Acid generator | Quencher | Organic solvent | temperature | Sensitivity | CDU |
| | (parts by mass) | (parts by mass) | (parts bymass) | (parts by mass) | (° C.) | (mJ/cm²) | (nm) |
| Ex. 22 | P-1 (100) | PAG-3 (25.7) | Q-18 (4.82) | PGMEA(3,000) | 80 | 31 | 3.1 |
| | | | | DAA(500) | | | |
| Ex. 23 | P-1 (100) | PAG-3 (25.7) | Q-19 (4.82) | PGMEA(3,000) | 80 | 31 | 3.0 |
| | | | | DAA(500) | | | |
| Ex. 24 | P-1 (100) | PAG-3 (25.7) | Q-20 (4.82) | PGMEA(3,000) | 80 | 32 | 3.0 |
| | | | | DAA(500) | | | |
| Ex. 25 | P-1 (100) | PAG-3 (25.7) | Q-21 (4.66) | PGMEA(3,000) | 80 | 32 | 3.0 |
| | | | | DAA(500) | | | |
| Ex. 26 | P-1 (100) | PAG-3 (25.7) | Q-22 (4.68) | PGMEA(3,000) | 80 | 33 | 3.3 |
| | | | | DAA(500) | | | |
| Ex. 27 | P-1 (100) | PAG-3 (25.7) | Q-23 (4.66) | PGMEA(3,000) | 80 | 31 | 3.2 |
| | | | | DAA(500) | | | |
| Ex. 28 | P-1 (100) | PAG-3 (25.7) | Q-24 (5.38) | PGMEA(3,000) | 80 | 32 | 3.2 |
| | | | | DAA(500) | | | |
| Ex. 29 | P-1 (100) | PAG-3 (25.7) | Q-25 (5.22) | PGMEA(3,000) | 80 | 31 | 3.0 |
| | | | | DAA(500) | | | |
| Ex. 30 | P-1 (100) | PAG-3 (25.7) | Q-26 (5.20) | PGMEA(3,000) | 80 | 33 | 3.2 |
| | | | | DAA(500) | | | |
| Ex. 31 | P-1 (100) | PAG-3 (25.7) | Q-27 (5.20) | PGMEA(3,000) | 80 | 30 | 3.1 |
| | | | | DAA(500) | | | |
| Ex. 32 | P-1 (100) | PAG-3 (25.7) | Q-28 (6.13) | PGMEA(3,000) | 80 | 32 | 3.1 |
| | | | | DAA(500) | | | |

From the results shown in Table 1 and Table 2, it has been found that the inventive resist material has high sensitivity and improved CDU, containing, as a quencher, a sulfonium salt of a substituted or unsubstituted hydroxy(trifluoromethoxy)benzoic acid; and/or a sulfonium salt of a substituted or unsubstituted hydroxy(trifluoromethylthio)benzoic acid, or one or more of: a sulfonium salt of a substituted or unsubstituted hydroxy(difluoromethoxy)benzoic acid; a sulfonium salt of a substituted or unsubstituted hydroxy(difluoromethylthio)benzoic acid; a sulfonium salt of a 2- or 3-, di- or tri-fluoromethoxybenzoic acid; and a sulfonium salt of a 2- or 3-, di- or tri-fluoromethylthiobenzoic acid.

The present specification includes the following aspects.

[1]: A resist material, comprising, as a quencher: a sulfonium salt of a substituted or unsubstituted hydroxy(trifluoromethoxy)benzoic acid; and/or a sulfonium salt of a substituted or unsubstituted hydroxy(trifluoromethylthio)benzoic acid.

[2]: The resist material according to [1], wherein the sulfonium salt of the substituted or unsubstituted hydroxy(trifluoromethoxy)benzoic acid and/or the sulfonium salt of the substituted or unsubstituted hydroxy(trifluoromethylthio)benzoic acid is represented by the following general formula (1),

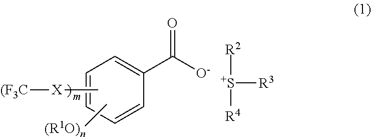

wherein R¹ represents a hydrogen atom or a group selected from a linear, branched, or cyclic alkyl group, alkoxy group, alkoxycarbonyl group, or acyl group having 1 to 15 carbon atoms, an alkenyl group having 2 to 15 carbon atoms, and an alkynyl group having 2 to 15 carbon atoms, the group optionally having a halogen atom, a carbonyl group, an ether bond, a substituted or unsubstituted aryl group, or a hydroxy group, the group being optionally an acid-labile group; X represents an oxygen atom or a sulfur atom; “m” and “n” each represent 1 or 2; and R² to R⁴ each independently represent a halogen atom or a hydrocarbyl group having 1 to 25 carbon atoms and optionally having a heteroatom, R² and R³ being optionally bonded to each other to form a ring together with the sulfur atom to which these groups are bonded.

[3]: A resist material, comprising, as a quencher, one or more of: a sulfonium salt of a substituted or unsubstituted hydroxy(difluoromethoxy)benzoic acid; a sulfonium salt of a substituted or unsubstituted hydroxy(difluoromethylthio)benzoic acid; a sulfonium salt of a 2- or 3-, di- or tri-fluoromethoxybenzoic acid; and a sulfonium salt of a 2- or 3-, di- or tri-fluoromethylthiobenzoic acid.

[4]: The resist material according to [3], wherein one or more of the sulfonium salt of the substituted or unsubstituted hydroxy(difluoromethoxy)benzoic acid, the sulfonium salt of the substituted or unsubstituted hydroxy(difluoromethylthio)benzoic acid, the sulfonium salt of the 2- or 3-, di- or tri-fluoromethoxybenzoic acid, and the sulfonium salt of the 2- or 3-, di- or tri-fluoromethylthiobenzoic acid are represented by the following general formula (1′),

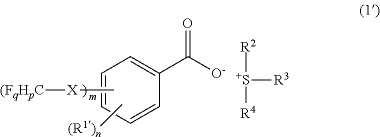

wherein R^{1′} represents a hydrogen atom, a halogen atom, or a group selected from a hydroxy group, a substituted or unsubstituted amino group, a linear, branched, or cyclic alkyl group, alkoxy group, alkoxycarbonyl group, alkoxycarbonyloxy group, acyl group, or acyloxy group having 1 to 15 carbon atoms, an alkenyloxy group having 2 to 15 carbon atoms, and an alkynyloxy group having 2 to 15 carbon atoms, the group optionally having a halogen atom, a carbonyl group, an ether bond, a substituted or unsubstituted aryl group, or a hydroxy group, the group being optionally an acid-labile group, and when R^{1′} represents a hydrogen atom, the substituting position of the F_{q}HₚC—X— group is the 2- or 3-position; “p” represents 0 or 1; “q” represents 2 or 3; X represents an oxygen atom or a sulfur atom; “m” and “n” each represent 1 or 2; and R² to R⁴ each independently represent a halogen atom or a hydrocarbyl group having 1 to 25 carbon atoms and optionally having a heteroatom, R² and R³ being optionally bonded to each other to form a ring together with the sulfur atom to which these groups are bonded.

[5]: The resist material according to any one of [1] to [4], further comprising an acid generator to generate an acid.

[6]: The resist material according to [5], wherein the acid generator generates a sulfonic acid, an imide acid, or a methide acid.

[7]: The resist material according to any one of [1] to [6], further comprising an organic solvent.

[8]: The resist material according to any one of [1] to [7], further comprising a base polymer.

[9]: The resist material according to [8], wherein the base polymer comprises: a repeating unit represented by the following general formula (a1) and/or a repeating unit represented by the following general formula (a2),

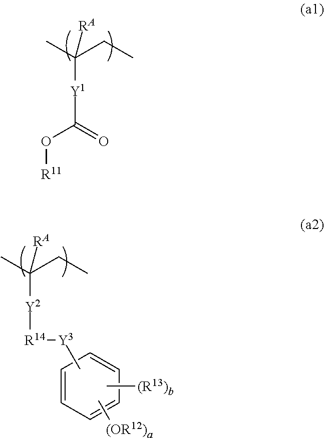

wherein R^{A} each independently represents a hydrogen atom or a methyl group; Y¹ represents a single bond, a phenylene group, a naphthylene group, or a linking group having 1 to 12 carbon atoms and having at least one selected from an ester bond and a lactone ring; Y² represents a single bond or an ester bond; Y³ represents a single bond, an ether bond, or an ester bond; R¹¹ and R¹² each independently represent an acid-labile group; R¹³ represents a fluorine atom, a trifluoromethyl group, a cyano group, or a saturated hydrocarbyl group having 1 to 6 carbon atoms; R¹⁴ represents a single bond or an alkanediyl group having 1 to 6 carbon atoms, a part of carbon atoms therein being optionally substituted with an ether bond or an ester bond; “a” represents 1 or 2; “b” represents an integer of 0 to 4, and 1≤a+b≤5.

[10]: The resist material according to [9], wherein the resist material is a chemically amplified positive-type resist material.

[11]: The resist material according to [8], wherein the base polymer has no acid-labile group.

[12]: The resist material according to [11], wherein the resist material is a chemically amplified negative-type resist material.

[13]: The resist material according to any one of [8] to [12], wherein the base polymer further comprises at least one selected from repeating units represented by the following general formulae (f1) to (f3),

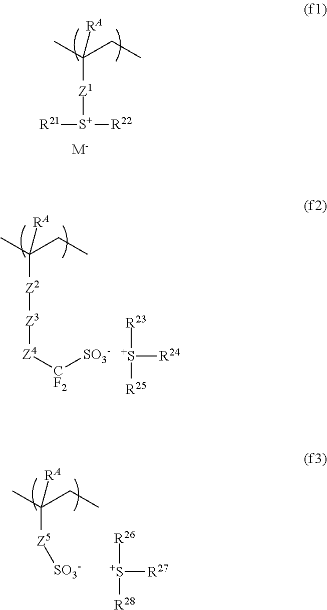

wherein R^{A} each independently represents a hydrogen atom or a methyl group; Z¹ represents a single bond, an aliphatic hydrocarbylene group having 1 to 6 carbon atoms, a phenylene group, a naphthylene group, an ester bond, a group having 7 to 18 carbon atoms obtained by combining these groups, —O—Z¹¹—, —C(═O)— or —C(═O)—NH—Z¹¹—; Z″ represents an aliphatic hydrocarbylene group having 1 to 6 carbon atoms, a phenylene group, a naphthylene group, or a group having 7 to 18 carbon atoms obtained by combining these groups, Z″ optionally having a carbonyl group, an ester bond, an ether bond, or a hydroxy group; Z² represents a single bond or an ester bond; Z³ represents a single bond, —Z³¹—C(═O)—O—, —Z³¹—O—, or —Z³¹—O—C(═O)—; Z³¹ represents a hydrocarbylene group having 1 to 12 carbon atoms, a phenylene group, or a group having 7 to 18 carbon atoms obtained by combining these groups, Z³¹ optionally having a carbonyl group, an ester bond, an ether bond, an iodine atom, or a bromine atom; Z⁴ represents a methylene group, a 2,2,2-trifluoro-1,1-ethanediyl group, or a carbonyl group; Z⁵ represents a single bond, a methylene group, an ethylene group, a phenylene group, a fluorinated phenylene group, a phenylene group substituted with a trifluoromethyl group, —O—Z⁵¹—, —C(═O)—O—Z⁵¹—, or —C(═O)—NH—Z⁵¹—; Z⁵¹ represents an aliphatic hydrocarbylene group having 1 to 15 carbon atoms, a phenylene group, a fluorinated phenylene group, a phenylene group substituted with a trifluoromethyl group, or a combination thereof, Z⁵¹ optionally having a carbonyl group, an ester bond, an ether bond, a halogen atom, and/or a hydroxy group; R²¹ to R²⁸ each independently represent a halogen atom or a hydrocarbyl group having 1 to 25 carbon atoms and optionally having a heteroatom; R²³ and R²⁴ or R²⁶ and R²⁷ are optionally bonded to each other to form a ring together with the sulfur atom to which these groups are bonded; and M⁻ represents a non-nucleophilic counterion.

[14]: The resist material according to any one of [1] to [13], further comprising a surfactant.

[15]: A patterning process, comprising steps of: forming a resist film on a substrate by using the resist material according to any one of [1] to [14]; exposing the resist film to high-energy ray; and developing the exposed resist film by using a developer.

[16]: The patterning process according to [15], wherein KrF excimer laser light, ArF excimer laser light, electron beam, or extreme ultraviolet ray having a wavelength of 3 to 15 nm is used as the high-energy ray.

It should be noted that the present invention is not limited to the above-described embodiments. The embodiments are just examples, and any examples that substantially have the same feature and demonstrate the same functions and effects as those in the technical concept disclosed in claims of the present invention are included in the technical scope of the present invention.

1. A resist material, comprising, as a quencher:
a sulfonium salt of a substituted or unsubstituted hydroxy(trifluoromethoxy)benzoic acid; and/or
a sulfonium salt of a substituted or unsubstituted hydroxy(trifluoromethylthio)benzoic acid.

2. The resist material according to claim 1, wherein the sulfonium salt of the substituted or unsubstituted hydroxy(trifluoromethoxy)benzoic acid and/or the sulfonium salt of the substituted or unsubstituted hydroxy(trifluoromethylthio)benzoic acid is represented by the following general formula (1), 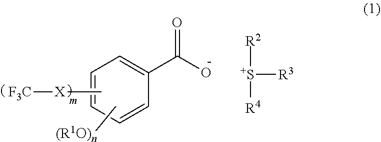
wherein R¹ represents a hydrogen atom or a group selected from a linear, branched, or cyclic alkyl group, alkoxy group, alkoxycarbonyl group, or acyl group having 1 to 15 carbon atoms, an alkenyl group having 2 to 15 carbon atoms, and an alkynyl group having 2 to 15 carbon atoms, the group optionally having a halogen atom, a carbonyl group, an ether bond, a substituted or unsubstituted aryl group, or a hydroxy group, the group being optionally an acid-labile group; X represents an oxygen atom or a sulfur atom; “m” and “n” each represent 1 or 2; and R² to R⁴ each independently represent a halogen atom or a hydrocarbyl group having 1 to 25 carbon atoms and optionally having a heteroatom, R² and R³ being optionally bonded to each other to form a ring together with the sulfur atom to which these groups are bonded.

3. A resist material, comprising, as a quencher, one or more of:
a sulfonium salt of a substituted or unsubstituted hydroxy(difluoromethoxy)benzoic acid;
a sulfonium salt of a substituted or unsubstituted hydroxy(difluoromethylthio)benzoic acid;
a sulfonium salt of a 2- or 3-, di- or tri-fluoromethoxybenzoic acid; and
a sulfonium salt of a 2- or 3-, di- or tri-fluoromethylthiobenzoic acid.

4. The resist material according to claim 3, wherein one or more of the sulfonium salt of the substituted or unsubstituted hydroxy(difluoromethoxy)benzoic acid, the sulfonium salt of the substituted or unsubstituted hydroxy(difluoromethylthio)benzoic acid, the sulfonium salt of the 2- or 3-, di- or tri-fluoromethoxybenzoic acid, and the sulfonium salt of the 2- or 3-, di- or tri-fluoromethylthiobenzoic acid are represented by the following general formula (1′), 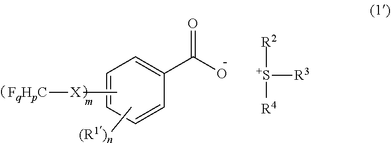
wherein R^{1′} represents a hydrogen atom, a halogen atom, or a group selected from a hydroxy group, a substituted or unsubstituted amino group, a linear, branched, or cyclic alkyl group, alkoxy group, alkoxycarbonyl group, alkoxycarbonyloxy group, acyl group, or acyloxy group having 1 to 15 carbon atoms, an alkenyloxy group having 2 to 15 carbon atoms, and an alkynyloxy group having 2 to 15 carbon atoms, the group optionally having a halogen atom, a carbonyl group, an ether bond, a substituted or unsubstituted aryl group, or a hydroxy group, the group being optionally an acid-labile group, and when R^{1′} represents a hydrogen atom, the substituting position of the F_{q}HₚC—X— group is the 2- or 3-position; “p” represents 0 or 1; “q” represents 2 or 3; X represents an oxygen atom or a sulfur atom; “m” and “n” each represent 1 or 2; and R² to R⁴ each independently represent a halogen atom or a hydrocarbyl group having 1 to carbon atoms and optionally having a heteroatom, R² and R³ being optionally bonded to each other to form a ring together with the sulfur atom to which these groups are bonded.

5. The resist material according to claim 1, further comprising an acid generator to generate an acid.

6. The resist material according to claim 2, further comprising an acid generator to generate an acid.

7. The resist material according to claim 5, wherein the acid generator generates a sulfonic acid, an imide acid, or a methide acid.

8. The resist material according to claim 1, further comprising an organic solvent.

9. The resist material according to claim 2, further comprising an organic solvent.

10. The resist material according to claim 5, further comprising an organic solvent.

11. The resist material according to claim 1, further comprising a base polymer.

12. The resist material according to claim 2, further comprising a base polymer.

13. The resist material according to claim 5, further comprising a base polymer.

14. The resist material according to claim 8, further comprising a base polymer.

15. The resist material according to claim 11, wherein the base polymer comprises: a repeating unit represented by the following general formula (a1) and/or a repeating unit represented by the following general formula (a2), 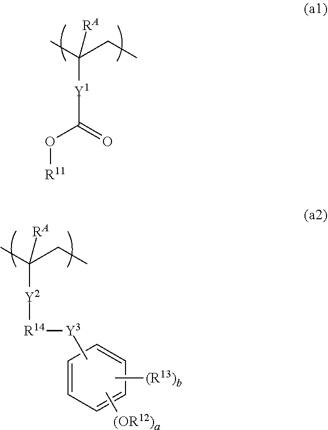
wherein R^{A} each independently represents a hydrogen atom or a methyl group; Y¹ represents a single bond, a phenylene group, a naphthylene group, or a linking group having 1 to 12 carbon atoms and having at least one selected from an ester bond and a lactone ring; Y² represents a single bond or an ester bond; Y³ represents a single bond, an ether bond, or an ester bond; R¹¹ and R¹² each independently represent an acid-labile group; R¹³ represents a fluorine atom, a trifluoromethyl group, a cyano group, or a saturated hydrocarbyl group having 1 to 6 carbon atoms; R¹⁴ represents a single bond or an alkanediyl group having 1 to 6 carbon atoms, a part of carbon atoms therein being optionally substituted with an ether bond or an ester bond; “a” represents 1 or 2; “b” represents an integer of 0 to 4, and 1≤a+b≤5.

16. The resist material according to claim 15, wherein the resist material is a chemically amplified positive-type resist material.

17. The resist material according to claim 11, wherein the base polymer has no acid-labile group.

18. The resist material according to claim 17, wherein the resist material is a chemically amplified negative-type resist material.

19. The resist material according to claim 11, wherein the base polymer further comprises at least one selected from repeating units represented by the following general formulae (f1) to (f3), 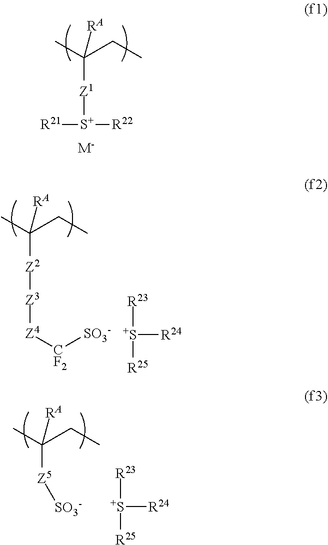
wherein R^{A} each independently represents a hydrogen atom or a methyl group; Z¹ represents a single bond, an aliphatic hydrocarbylene group having 1 to 6 carbon atoms, a phenylene group, a naphthylene group, an ester bond, a group having 7 to 18 carbon atoms obtained by combining these groups, —O—Z¹¹—, —C(═O)— or —C(═O)—NH—Z″—; Z″ represents an aliphatic hydrocarbylene group having 1 to 6 carbon atoms, a phenylene group, a naphthylene group, or a group having 7 to 18 carbon atoms obtained by combining these groups, Z″ optionally having a carbonyl group, an ester bond, an ether bond, or a hydroxy group; Z² represents a single bond or an ester bond; Z³ represents a single bond, —Z³¹—C(═O)—O—, —Z³¹—O—, or —Z³¹—O—C(═O)—; Z³¹ represents a hydrocarbylene group having 1 to 12 carbon atoms, a phenylene group, or a group having 7 to 18 carbon atoms obtained by combining these groups, Z³¹ optionally having a carbonyl group, an ester bond, an ether bond, an iodine atom, or a bromine atom; Z⁴ represents a methylene group, a 2,2,2-trifluoro-1,1-ethanediyl group, or a carbonyl group; Z⁵ represents a single bond, a methylene group, an ethylene group, a phenylene group, a fluorinated phenylene group, a phenylene group substituted with a trifluoromethyl group, —O—Z⁵¹—, —C(═O)—O—Z⁵¹—, or —C(═O)—NH—Z⁵¹—; Z⁵¹ represents an aliphatic hydrocarbylene group having 1 to 15 carbon atoms, a phenylene group, a fluorinated phenylene group, a phenylene group substituted with a trifluoromethyl group, or a combination thereof, Z⁵¹ optionally having a carbonyl group, an ester bond, an ether bond, a halogen atom, and/or a hydroxy group; R²¹ to R²⁸ each independently represent a halogen atom or a hydrocarbyl group having 1 to 25 carbon atoms and optionally having a heteroatom; R²³ and R²⁴ or R²⁶ and R²⁷ are optionally bonded to each other to form a ring together with the sulfur atom to which these groups are bonded; and M⁻ represents a non-nucleophilic counterion.

20. The resist material according to claim 1, further comprising a surfactant.

21. A patterning process, comprising steps of:
forming a resist film on a substrate by using the resist material according to claim 1;
exposing the resist film to high-energy ray; and
developing the exposed resist film by using a developer.

22. The patterning process according to claim 21, wherein KrF excimer laser light, ArF excimer laser light, electron beam, or extreme ultraviolet ray having a wavelength of 3 to 15 nm is used as the high-energy ray.
